(19) United States
(12) Patent Application Publication
(10) Pub. No.: US 2017/0001988 A1
(43) Pub. Date: Jan. 5, 2017

(54) PIPERIDINE AND PIPERAZINE DERIVATIVES AND THEIR USE IN TREATING VIRAL INFECTIONS AND CANCER

(71) Applicant: THE UNITED STATES OF AMERICA, AS REPRESENTED BY THE SECRETARY, DEPARTMENT OF HEALTH AND HUMAN, Bethesda, MD (US)

(72) Inventors: Tsanyang Jake Liang, Potomac, MD (US); Marc Ferrer, Potomac, MD (US); Shanshan He, North Bethesda, MD (US); Xin Hu, Frederick, MD (US); Zongyi Hu, Potomac, MD (US); Juan Jose Marugan, Gaithersburg, MD (US); Noel Terrence Southall, Potomac, MD (US); Jingbo Xiao, Rockville, MD (US); Wei Zheng, Potomac, MD (US)

(21) Appl. No.: 15/039,781

(22) PCT Filed: Nov. 20, 2014
(86) PCT No.: PCT/US2014/066680

§ 371 Date: May 26, 2016

Related U.S. Application Data
(60) Provisional application No. 61/909,414, filed on Nov. 27, 2013.

(51) Int. Cl. C07D 405/10; A61K 31/55; A61K 45/06; C07D 211/58; A61K 31/453; A61K 31/4468; C07D 295/073; A61K 31/495; C07D 213/16; A61K 31/4409; A61K 31/4465; C07D 211/12; C07D 295/03; C07D 211/14; C07D 403/12; A61K 31/496; C07D 243/08

(52) U.S. Cl. CPC C07D 405/10; C07D 243/08; A61K 31/55; A61K 45/06; C07D 211/58; A61K 31/453; A61K 31/4468; C07D 295/073; A61K 31/495; C07D 213/16; A61K 31/4409; A61K 31/4465; C07D 211/12; C07D 295/03; C07D 211/14; C07D 403/12; A61K 31/496

(57) ABSTRACT

Disclosed are compounds of formula (I) (formula I), as antiviral agents, antineoplastic agents, pharmaceutical compositions comprising such compounds, and a method of use of these compounds, wherein X and Y are independently CH or N, o is 0, 1 or 2, and E is absent or is (CR¹³R¹⁴)m, NH, or S, F is absent or is (CR¹⁵R¹⁶)n, C=O, or —SO₂—, G is absent or is (CR¹⁷ CR¹⁸)r, H is absent or is C═O, or —SO2- and R¹, Ar¹, Ar² are as defined in the specification. These compounds are antiviral agents and are contemplated in the treatment of viral infections, for example, hepatitis C, or are antineoplastic agents.

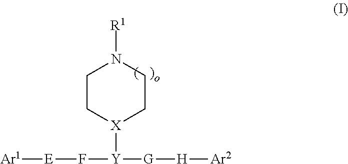

### CROSS-REFERENCE TO A RELATED APPLICATION

This patent application claims the benefit of U.S. Provisional Patent Application No. 61/909,414, filed Nov. 27, 2013, which is incorporated by reference.

### BACKGROUND OF THE INVENTION

Hepatitis C virus (HCV) infects about 200 million people in the world. Many infected people progress to chronic liver disease including cirrhosis with a risk of developing liver cancer. To date, there is no effective vaccine for hepatitis C.

Current standard treatment of chronic hepatitis C, based on combination of peginterferon-α and ribavirin, is only effective in about half of the patients, with significant adverse effects. The fraction of people with HCV who can complete a successful treatment is estimated to be no more than 10 percent. Recent development of direct-acting antivirals against HCV, such as protease and polymerase inhibitors, is promising but still requires combination with peginterferon and ribavirin for maximal efficacy. In addition, these agents are associated with high rate of resistance and many have significant side effects.

In view of the foregoing, an unmet need exists for novel agents for treating or preventing viral infection.

### BRIEF SUMMARY OF THE INVENTION

The invention provides a compound of formula (I):

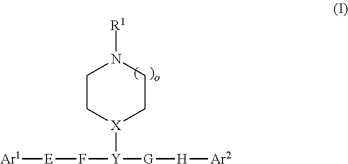

wherein R¹ is selected from hydrogen, C₁-C₁₀ alkyl, C₃-C₁₀ cycloalkyl, C₃-C₁₀ cycloalkyl C₁-C₁₀ alkyl, C₆-C₁₀ aryl, C₆-C₁₀ aryl C₁-C₁₀ alkyl, C₆-C₁₀ aryl C₃-C₁₀ cycloalkyl, heteroaryl, heterocyclyl, C₆₋₁₀ arylsulfonyl, C₆₋₁₀ arylcarbonyl, C₁-C₁₀ alkylcarbonyl, —(CH₂)ₓA(CH₂)_{y}B, and —(CH₂CH₂O)ₚ(CH₂CH₂)_{q}P, wherein the alkyl, aryl, or heteroaryl part of R¹ is optionally substituted with one or more substituents selected from deuterium, halo, C₁-C₁₀ alkyl, C₆-C₁₀ aryl, trifluoromethyl, C₁-C₁₀ alkoxy, cyano, alkylenedioxy, C₁-C₁₀ alkylcarbonyl, and C₁-C₁₀ alkoxycarbonyl,

Ar¹ and Ar² are the same or different and are independently selected from C₆-C₁₀ aryl, heteroaryl, and heterocyclyl, wherein the aryl, heteroaryl, and heterocyclyl are optionally substituted with one or more substituents selected from halo, C₁-C₁₀ alkyl, C₆-C₁₀ aryl, trifluoromethyl, C₁-C₁₀ alkoxy, C₁-C₁₀ alkylcarbonyl, and C₁-C₁₀ alkoxycarbonyl,

A is O, S, or N,

x and y are independently 1-4, inclusive,

B is selected from OR⁴, COOR⁵, and CONR⁶R⁷,

wherein R⁴, R⁵, R⁶, and R⁷ are independently selected from hydrogen, C₁-C₁₀ alkyl, C₃-C₁₀ cycloalkyl, and C₆-C₁₀ aryl,

D is NR⁸R⁹, OH, or OR¹²,

R⁸ and R⁹ are independently selected from hydrogen, COR¹⁰, and COOR¹¹,

R¹⁰ and R¹¹ are hydrogen or C₁-C₁₀ alkyl,

p and q are independently 1-4, inclusive,

E is absent or is (CR¹³R¹⁴)ₘ, NH, or S,

F is absent or is (CR¹⁵R¹⁶)ₙ, C═O, or —SO2-,

G is absent or is (CR17CR18)r,

H is absent or is C═O, or —SO2-,

M, n, and r are independently 0, 1, 2, 3, or 4,

o is 0, 1, or 2,

X and Y are independently CH or N,

or pharmaceutically acceptable salts, stereoisomers, and mixtures comprising stereoisomers thereof.

The invention also provides a method of treating or preventing hepatitis C comprising administering to a mammal in need thereof an effective amount of a compound of formula (I):

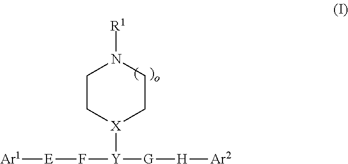

wherein R¹ is selected from hydrogen, C₁-C₁₀ alkyl, C₃-C₁₀ cycloalkyl, C₃-C₁₀ cycloalkyl C₁-C₁₀ alkyl, C₆-C₁₀ aryl, C₆-C₁₀ aryl C₁-C₁₀ alkyl, C₆-C₁₀ aryl C₃-C₁₀ cycloalkyl, heteroaryl, heterocyclyl, C₆₋₁₀ arylsulfonyl, C₆₋₁₀ arylcarbonyl, C₁-C₁₀ alkylcarbonyl, —(CH₂)ₓA(CH₂)_{y}B, and —(CH₂CH₂O)ₚ(CH₂CH₂)_{q}D, wherein the alkyl, aryl, or heteroaryl part of R¹ is optionally substituted with one or more substituents selected from deuterium, halo, C₁-C₁₀ alkyl, C₆-C₁₀ aryl, trifluoromethyl, C₁-C₁₀ alkoxy, cyano, alkylenedioxy, C₁-C₁₀ alkylcarbonyl, and C₁-C₁₀ alkoxycarbonyl,

Ar¹ and Ar² are the same or different and are independently selected from C₆-C₁₀ aryl, heteroaryl, and heterocyclyl, wherein the aryl, heteroaryl, and heterocyclyl are optionally substituted with one or more substituents selected from halo, C₁-C₁₀ alkyl, C₆-C₁₀ aryl, trifluoromethyl, C₁-C₁₀ alkoxy, C₁-C₁₀ alkylcarbonyl, and C₁-C₁₀ alkoxycarbonyl,

A is O, S, or N,

x and y are independently 1-4, inclusive,

B is selected from OR⁴, COOR⁵, and CONR⁶R⁷,

wherein R⁴, R⁵, R⁶, and R⁷ are independently selected from hydrogen, C₁-C₁₀ alkyl, C₃-C₁₀ cycloalkyl, and C₆-C₁₀ aryl,

D is NR⁸R⁹, OH, or OR¹²,

R⁸ and R⁹ are independently selected from hydrogen, COR¹⁰, and COOR¹¹,

R¹⁰ and R¹¹ are hydrogen or C₁-C₁₀ alkyl,

p and q are independently 1-4, inclusive,

E is absent or is (CR¹³R¹⁴)ₘ, NH, or S,

F is absent or is (CR¹⁵R¹⁶)ₙ, C═O, or —SO2-,

G is absent or is (CR17CR18)r,

H is absent or is C═O, or —SO2-,

M, n, and r are independently 0, 1, 2, 3, or 4,

o is 0, 1, or 2,

X and Y are independently CH or N,

or pharmaceutically acceptable salts, stereoisomers, and mixtures comprising stereoisomers thereof

The invention further provides a method for synergistically enhancing the antiviral effect of an anti-hepatitis C compound in a mammal undergoing treatment with the anti-hepatitis C compound, comprising administering to the mammal a compound of the formula (I):

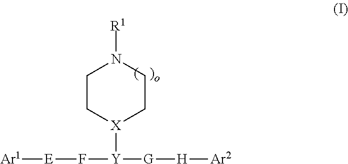

wherein R¹ is selected from hydrogen, C₁-C₁₀ alkyl, C₃-C₁₀ cycloalkyl, C₃-C₁₀ cycloalkyl C₁-C₁₀ alkyl, C₆-C₁₀ aryl, C₆-C₁₀ aryl C₁-C₁₀ alkyl, C₆-C₁₀ aryl C₃-C₁₀ cycloalkyl, heteroaryl, heterocyclyl, C₆₋₁₀ arylsulfonyl, C₆₋₁₀ arylcarbonyl, C₁-C₁₀ alkylcarbonyl, —(CH₂)ₓA(CH₂)_{y}B, and —(CH₂CH₂O)ₚ(CH₂CH₂)_{q}D, wherein the alkyl, aryl, or heteroaryl part of R¹ is optionally substituted with one or more substituents selected from deuterium, halo, C₁-C₁₀ alkyl, C₆-C₁₀ aryl, trifluoromethyl, C₁-C₁₀ alkoxy, cyano, alkylenedioxy, C₁-C₁₀ alkylcarbonyl, and C₁-C₁₀ alkoxycarbonyl,

Ar¹ and Ar² are the same or different and are independently selected from C₆-C₁₀ aryl, heteroaryl, and heterocyclyl, wherein the aryl, heteroaryl, and heterocyclyl are optionally substituted with one or more substituents selected from halo, C₁-C₁₀ alkyl, C₆-C₁₀ aryl, trifluoromethyl, C₁-C₁₀ alkoxy, C₁-C₁₀ alkylcarbonyl, and C₁-C₁₀ alkoxycarbonyl,

A is O, S, or N,

x and y are independently 1-4, inclusive,

B is selected from OR⁴, COOR⁵, and CONR⁶R⁷,

wherein R⁴, R⁵, R⁶, and R⁷ are independently selected from hydrogen, C₁-C₁₀ alkyl, C₃-C₁₀ cycloalkyl, and C₆-C₁₀ aryl,

D is NR⁸R⁹, OH, or OR¹²,

R⁸ and R⁹ are independently selected from hydrogen, COR¹⁰, and COOR¹¹,

R¹⁰ and R¹¹ are hydrogen or C₁-C₁₀ alkyl,

p and q are independently 1-4, inclusive,

E is absent or is (CR¹³R¹⁴)ₘ, NH, or S,

F is absent or is (CR¹⁵R¹⁶)ₙ, C═O, or —SO2-,

G is absent or is (CR17CR18)r,

H is absent or is C═O, or —SO2-,

M, n, and r are independently 0, 1, 2, 3, or 4,

o is 0, 1, or 2,

X and Y are independently CH or N,

or pharmaceutically acceptable salts, stereoisomers, and mixtures comprising stereoisomers thereof, in combination with the anti-hepatitis C compound.

The invention additionally provides a kit comprising:

(a) a compound of formula (I):

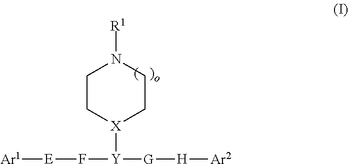

wherein R¹ is selected from hydrogen, C₁-C₁₀ alkyl, C₃-C₁₀ cycloalkyl, C₃-C₁₀ cycloalkyl C₁-C₁₀ alkyl, C₆-C₁₀ aryl, C₆-C₁₀ aryl C₁-C₁₀ alkyl, C₆-C₁₀ aryl C₃-C₁₀ cycloalkyl, heteroaryl, heterocyclyl, C₆₋₁₀ arylsulfonyl, C₆₋₁₀ arylcarbonyl, C₁-C₁₀ alkylcarbonyl, —(CH₂)ₓA(CH₂)_{y}B, and —(CH₂CH₂O)ₚ(CH₂CH₂)_{q}D, wherein the alkyl, aryl, or heteroaryl part of R¹ is optionally substituted with one or more substituents selected from deuterium, halo, C₁-C₁₀ alkyl, C₆-C₁₀ aryl, trifluoromethyl, C₁-C₁₀ alkoxy, cyano, alkylenedioxy, C₁-C₁₀ alkylcarbonyl, and C₁-C₁₀ alkoxycarbonyl,

Ar¹ and Ar² are the same or different and are independently selected from C₆-C₁₀ aryl, heteroaryl, and heterocyclyl, wherein the aryl, heteroaryl, and heterocyclyl are optionally substituted with one or more substituents selected from halo, C₁-C₁₀ alkyl, C₆-C₁₀ aryl, trifluoromethyl, C₁-C₁₀ alkoxy, C₁-C₁₀ alkylcarbonyl, and C₁-C₁₀ alkoxycarbonyl,

A is O, S, or N,

x and y are independently 1-4, inclusive,

B is selected from OR⁴, COOR⁵, and CONR⁶R⁷,

wherein R⁴, R⁵, R⁶, and R⁷ are independently selected from hydrogen, C₁-C₁₀ alkyl, C₃-C₁₀ cycloalkyl, and C₆-C₁₉ aryl,

D is NR⁸R⁹, OH, or OR¹²,

R⁸ and R⁹ are independently selected from hydrogen, COR¹⁰, and COOR¹¹,

R¹⁹ and R¹¹ are hydrogen or C₁-C₁₀ alkyl,

p and q are independently 1-4, inclusive,

E is absent or is (CR¹³R¹⁴)ₘ, NH, or S,

F is absent or is (CR¹⁵R¹⁶)ₙ, C═O, or —SO2-,

G is absent or is (CR17CR18)r,

H is absent or is C═O or —SO2-,

M, n, and r are independently 0, 1, 2, 3, or 4,

o is 0, 1, or 2,

X and Y are independently CH or N,

or pharmaceutically acceptable salts, stereoisomers, and mixtures comprising stereoisomers thereof, and

(b) an anti-hepatitis C compound other than a compound of formula (II).

In accordance with an embodiment, extracellular and intracellular viral RNA levels were reduced with the treatment of compounds of the invention.

In accordance with an embodiment, inhibition of viral entry is not the mechanism of anti-HCV action of compounds of the invention.

In accordance with an embodiment, compounds of the invention exhibit synergistic antiviral effect of chlorcyclizine (“CCZ”) with current anti-HCV drugs, either approved or under clinical trial.

In accordance with an embodiment, compounds of the invention exhibit a lack of long-term in vitro cytotoxicity of chlorcyclizine hydrochloride.

In accordance with an embodiment, compounds of formula (I), for example, NCGC00345021, target the late stage of the HCV life cycle.

In accordance with an embodiment, inhibition of Dengue virus infection is produced by a compound of formula (I).

In accordance with an embodiment, inhibition of HCV genotype 1b and 2a infections in vivo is produced by a compound of formula (I) without clear evidence of drug resistance.

### BRIEF DESCRIPTION OF THE SEVERAL VIEWS OF THE DRAWING(S)

FIGS. 1A and 1B illustrate the reduction of extracellular and intracellular viral RNA levels, respectively, upon treatment with DMSO (vehicle), racemic chlorcyclizine hydrochloride (“CCZ”), (R)-CCZ, and (S)-CCZ. Cyclosporin A is included as a comparison. A=DMSO; B=racemic CCZ; C=(R)-CCZ; D=(S)-CCZ; E=Cyclosporin A.

FIG. 2A illustrates luciferase activity of Huh 7.5.1 cells that were inoculated with the infectious HCVsc virus together with DMSO (vehicle), racemic CCZ, (R)-CCZ, and (S)-CCZ, and Cyclosporin A. A=DMSO; B=racemic CCZ; C=(R)-CCZ; D=(S)-CCZ; E=Cyclosporin A.

FIG. 2B illustrates luciferase activity of HCV replicon GT 1b and 2a cells and transient replicon GT 1a cells that were treated with DMSO (vehicle), racemic CCZ, (R)-CCZ, and (S)-CCZ, and Cyclosporin A. A=DMSO; B=racemic CCZ; C=(R)-CCZ; D=(S)-CCZ; E=Cyclosporin A.

FIG. 2C illustrates luciferase activity of Huh 7.5.1 cells treated with DMSO (vehicle), racemic CCZ, (R)-CCZ, and (S)-CCZ, and rottlerin (known inhibitor of HCV entry) together with infection of HCVppGT 1a, 1b, VSVpp, and MLVpp, followed by culturing for 48 h. A=DMSO; B=racemic CCZ; C=(R)-CCZ; D=(S)-CCZ; E=rottlerin.

FIG. 3 illustrates the cell viability (expressed as a percent) of Huh 7.5.1 cells treated with DMSO (vehicle), 1.0, 5.0, and 10 μM of (S)-CCZ and with 1.0, 5.0, and 10 μM of Cyclosporin A.

FIG. 4A illustrates the extracellular and intracellular HCV RNA levels of Huh 7.5.1 cells that were infected with HCVcc in the presence of 0.32, 1.0. 33.2, 10, and 32 μM of NCGC00345021, a compound in accordance with an embodiment of the invention, and 0.032, 0.10 0.32, 1.0, and 3.2 μM of Cyclosporin A.

FIG. 4B illustrates the TCID50 of naïve Huh 7.5.1 cells that were infected using medium collected in the HCVcc assay run using 0.32, 1.0, and 3.2 μM concentrations of NCGC00345021 and 0.032, 0.10 and 0.32 μM concentrations of Cyclosporin A.

FIG. 5 depicts the structure of NCGC00345021, a compound in accordance with an embodiment of the invention.

FIG. 6 illustrates the dose-response inhibition of Dengue reporter Virus particles upon treatment with NCGC00345021.

FIG. 7A illustrates the changes in the genotype 1b HCV titers from pretreatment baseline over a period of 8 weeks with 4 weeks of (S)-CCZ treatment and 4 weeks of follow-up without treatment. The serum albumin levels are also shown in FIG. 7A over the treatment period.

FIG. 7B illustrates the changes in the genotype 2a HCV titers from pretreatment baseline over a period of 8 weeks with 4 weeks of (S)-CCZ treatment and 4 weeks of follow-up without treatment. The serum albumin levels are also shown in FIG. 7B over the treatment period.

FIG. 8 shows the anti-HCV activity and selectivity for embodiments of the invention.

FIG. 9 shows the results of HCV replication cycle assays for representative embodiments of the invention.

FIG. 10 shows the in vitro pharmacokinetics for representative embodiments of the invention.

FIGS. 11-14 depict structures of compounds in accordance with an embodiment of the invention.

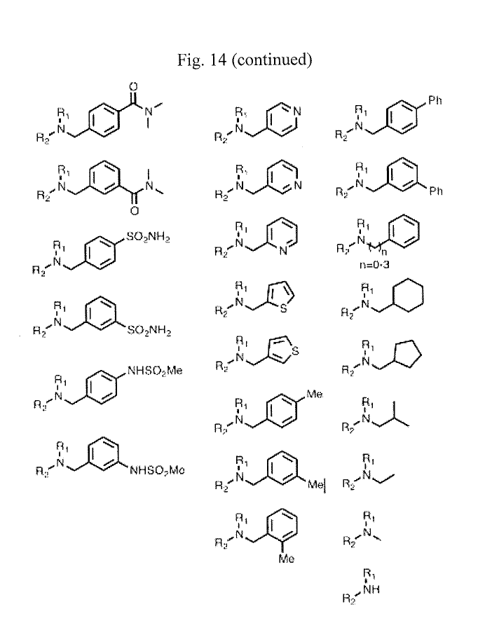

### DETAILED DESCRIPTION OF THE INVENTION

In an embodiment, the invention provides a compound of formula (I):

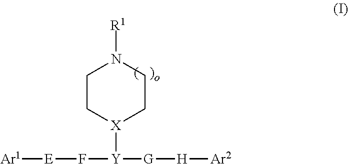

wherein R¹ is selected from hydrogen, C₁-C₁₀ alkyl, C₃-C₁₀ cycloalkyl, C₃-C₁₀ cycloalkyl C₁-C₁₀ alkyl, C₆-C₁₀ aryl, C₆-C₁₀ aryl C₁-C₁₀ alkyl, C₆-C₁₀ aryl C₃-C₁₀ cycloalkyl, heteroaryl, heterocyclyl, C₆₋₁₀ arylsulfonyl, C₆₋₁₀ arylcarbonyl, C₁-C₁₀ alkylcarbonyl, —(CH₂)ₓA(CH₂)_{y}B, and —(CH₂CH₂O)ₚ(CH₂CH₂)_{q}D, wherein the alkyl, aryl, or heteroaryl part of R¹ is optionally substituted with one or more substituents selected from deuterium, halo, C₁-C₁₀ alkyl, C₆-C₁₀ aryl, trifluoromethyl, C₁-C₁₀ alkoxy, cyano, alkylenedioxy, C₁-C₁₀ alkylcarbonyl, and C₁-C₁₀ alkoxycarbonyl,

Ar¹ and Ar² are the same or different and are independently selected from C₆-C₁₀ aryl, heteroaryl, and heterocyclyl, wherein the aryl, heteroaryl, and heterocyclyl are optionally substituted with one or more substituents selected from halo, C₁-C₁₀ alkyl, C₆-C₁₀ aryl, trifluoromethyl, C₁-C₁₀ alkoxy, C₁-C₁₀ alkylcarbonyl, and C₁-C₁₀ alkoxycarbonyl,

A is O, S, or N,

x and y are independently 1-4, inclusive,

B is selected from OR⁴, COOR⁵, and CONR⁶R⁷,

wherein R⁴, R⁵, R⁶, and R⁷ are independently selected from hydrogen, C₁-C₁₀ alkyl, C₃-C₁₀ cycloalkyl, and C₆-C₁₀ aryl,

D is NR⁸R⁹, OH, or OR¹²,

R⁸ and R⁹ are independently selected from hydrogen, COR¹⁰, and COOR¹¹,

R¹⁹ and R¹¹ are hydrogen or C₁-C₁₀ alkyl,

p and q are independently 1-4, inclusive,

E is absent or is (CR¹³R¹⁴)ₘ, NH, or S,

F is absent or is (CR¹⁵R¹⁶)ₙ, C═O, or —SO2-,

G is absent or is (CR17CR18)r,

H is absent or is C═O, or —SO2-,

M, n, and r are independently 0, 1, 2, 3, or 4,

o is 0, 1, or 2,

X and Y are independently CH or N,

or pharmaceutically acceptable salts, stereoisomers, and mixtures comprising stereoisomers thereof

with the provisos that (i) when E, F, G, and H are all absent, o is 1, X is N, Y is CH, and R¹ is hydrogen, methyl, ethyl, or isopropyl, the compound is a single enantiomer at the carbon bearing Ar¹ and Ar², and (ii) when E, F, G, and H are all absent, o is 1, X is CH and Y is N, R¹ is hydrogen, methyl, or ethyl.

Referring now to terminology used generically herein, the term “alkyl” means a straight-chain or branched alkyl substituent containing from, for example, 1 to about 6 carbon atoms, preferably from 1 to about 4 carbon atoms, more preferably from 1 to 2 carbon atoms. Examples of such substituents include methyl, ethyl, propyl, isopropyl, n-butyl, sec-butyl, isobutyl, tert-butyl, pentyl, isoamyl, hexyl, and the like.

The term “cycloalkyl,” as used herein, means a cyclic alkyl substituent containing from, for example, about 3 to about 8 carbon atoms, preferably from about 4 to about 7 carbon atoms, and more preferably from about 4 to about 6 carbon atoms. Examples of such substituents include cyclopropyl, cyclobutyl, cyclopentyl, cyclohexyl, cycloheptyl, cyclooctyl, and the like. The cyclic alkyl groups may be unsubstituted or further substituted with alkyl groups such as methyl groups, ethyl groups, and the like.

The term “heterocyclyl,” as used herein, refers to a monocyclic or bicyclic 5- or 6-membered ring system containing one or more heteroatoms selected from the group consisting of O, N, S, and combinations thereof. The heterocyclyl group can be any suitable heterocyclyl group and can be an aliphatic heterocyclyl group, an aromatic heterocyclyl group, or a combination thereof. The heterocyclyl group can be a monocyclic heterocyclyl group or a bicyclic heterocyclyl group. Suitable heterocyclyl groups include morpholine, piperidine, tetrahydrofuryl, oxetanyl, pyrrolidinyl, and the like. Suitable bicyclic heterocyclyl groups include monocylic heterocyclyl rings fused to a C₆-C₁₀ aryl ring. When the heterocyclyl group is a bicyclic heterocyclyl group, both ring systems can be aliphatic or aromatic, or one ring system can be aromatic and the other ring system can be aliphatic as in, for example, dihydrobenzofuran. The term “heteroaryl” refers to a monocyclic or bicyclic 5- or 6-membered ring system as described herein, wherein the heteroaryl group is unsaturated and satisfies Hackers rule. Non-limiting examples of suitable heteroaryl groups include furanyl, thiopheneyl, pyrrolyl, pyrazolyl, imidazolyl, 1,2,3-triazolyl, 1,2,4-triazolyl, isoxazolyl, oxazolyl, isothiazolyl, thiazolyl, 1,3,4-oxadiazol-2-yl, 1,2,4-oxadiazol-2-yl, 5-methyl-1,3,4-oxadiazole, 3-methyl-1,2,4-oxadiazole, pyridinyl, pyrimidinyl, pyrazinyl, pyridazinyl, triazinyl, benzofuranyl, benzothiopheneyl, indolyl, quinolinyl, isoquinolinyl, benzimidazolyl, benzoxazolinyl, benzothiazolinyl, and quinazolinyl. The heterocyclyl or heteroaryl group is optionally substituted with 1, 2, 3, 4, or 5 substituents as recited herein such as with alkyl groups such as methyl groups, ethyl groups, and the like, halo groups such as chloro, or hydroxyl groups, or with aryl groups such as phenyl groups, naphthyl groups and the like, wherein the aryl groups can be further substituted with, for example halo, dihaloalkyl, trihaloalkyl, nitro, hydroxy, alkoxy, aryloxy, amino, substituted amino, alkylcarbonyl, alkoxycarbonyl, arylcarbonyl, aryloxycarbonyl, thio, alkylthio, arylthio, and the like, wherein the optional substituent can be present at any open position on the heterocyclyl or heteroaryl group.

The term “alkylcarbonyl,” as used herein, refers to an alkyl group linked to a carbonyl group and further linked to a molecule via the carbonyl group, e.g., alkyl-C(═O)—. The term “alkoxycarbonyl,” as used herein, refers to an alkoxy group linked to a carbonyl group and further linked to a molecule via the carbonyl group, e.g., alkyl-O—C(═O)—.

The term “halo” or “halogen,” as used herein, means a substituent selected from Group VIIA, such as, for example, fluorine, bromine, chlorine, and iodine.

The term “aryl” refers to an unsubstituted or substituted aromatic carbocyclic substituent, as commonly understood in the art, and the term “C₆-C₁₀ aryl” includes phenyl and naphthyl. It is understood that the term aryl applies to cyclic substituents that are planar and comprise 4n+2 π electrons, according to Hackers Rule.

Whenever a range of the number of atoms in a structure is indicated (e.g., a C₁-C₁₂, C₁-C₈, C₁-C₆, C₁-C₄, or C₂-C₁₂, C₂-C₈, C₂-C₆, C₂-C₄ alkyl, alkenyl, alkynyl, etc.), it is specifically contemplated that any sub-range or individual number of carbon atoms falling within the indicated range also can be used. Thus, for instance, the recitation of a range of 1-8 carbon atoms (e.g., C₁-C₈), 1-6 carbon atoms (e.g., C₁-C₆), 1-4 carbon atoms (e.g., C₁-C₄), 1-3 carbon atoms (e.g., C₁-C₃), or 2-8 carbon atoms (e.g., C₂-C₈) as used with respect to any chemical group (e.g., alkyl, alkylamino, etc.) referenced herein encompasses and specifically describes 1, 2, 3, 4, 5, 6, 7, 8, 9, 10, 11, and/or 12 carbon atoms, as appropriate, as well as any sub-range thereof (e.g., 1-2 carbon atoms, 1-3 carbon atoms, 1-4 carbon atoms, 1-5 carbon atoms, 1-6 carbon atoms, 1-7 carbon atoms, 1-8 carbon atoms, 1-9 carbon atoms, 1-10 carbon atoms, 1-11 carbon atoms, 1-12 carbon atoms, 2-3 carbon atoms, 2-4 carbon atoms, 2-5 carbon atoms, 2-6 carbon atoms, 2-7 carbon atoms, 2-8 carbon atoms, 2-9 carbon atoms, 2-10 carbon atoms, 2-11 carbon atoms, 2-12 carbon atoms, 3-4 carbon atoms, 3-5 carbon atoms, 3-6 carbon atoms, 3-7 carbon atoms, 3-8 carbon atoms, 3-9 carbon atoms, 3-10 carbon atoms, 3-11 carbon atoms, 3-12 carbon atoms, 4-5 carbon atoms, 4-6 carbon atoms, 4-7 carbon atoms, 4-8 carbon atoms, 4-9 carbon atoms, 4-10 carbon atoms, 4-11 carbon atoms, and/or 4-12 carbon atoms, etc., as appropriate). Similarly, the recitation of a range of 6-10 carbon atoms (e.g., C₆-C₁₀) as used with respect to any chemical group (e.g., aryl) referenced herein encompasses and specifically describes 6, 7, 8, 9, and/or 10 carbon atoms, as appropriate, as well as any sub-range thereof (e.g., 6-10 carbon atoms, 6-9 carbon atoms, 6-8 carbon atoms, 6-7 carbon atoms, 7-10 carbon atoms, 7-9 carbon atoms, 7-8 carbon atoms, 8-10 carbon atoms, and/or 8-9 carbon atoms, etc., as appropriate).

In certain embodiments of the invention, X is CH and Y is N.

In certain embodiments, o is 1. In certain embodiments, m is 2. In certain embodiments, n is 1.

In certain embodiments, E is (CR¹³R¹⁴)ₘ, F is absent, and m is 2. In certain embodiments, H is absent and r is 1.

In certain embodiments, Ar¹ and Ar² are both phenyl.

In certain embodiments, R¹ is selected from C₁-C₁₀, alkyl, C₃-C₁₀ cycloalkyl, and C₃-C₁₀ cycloalkyl C₁-C₁₀ alkyl.

In certain preferred embodiments, R¹ is selected from hydrogen, cyclopentyl, sec-butyl, isopropyl, cyclohexyl, n-propyl, n-butyl, benzoyl, methyl, ethyl, trideuteromethyl, 2,2,2-trideuteroethyl, 2,2,2-trifluoroethyl, phenylsulfonyl, and benzyl.

In certain embodiments, R¹ is selected from C₆-C₁₀ aryl and C₆-C₁₀ aryl C₁-C₁₀ alkyl, wherein the aryl is optionally substituted with one or more substituents selected from halo, cyano, alkylenedioxy, C₁-C₁₀ alkyl, C₆-C₁₀ aryl, trifluoromethyl, C₁-C₁₀ alkoxy, cyano, alkylenedioxy, C₁-C₁₀ alkylcarbonyl, and C₁-C₁₀ alkoxycarbonyl.

In certain preferred embodiments, R¹ is selected from 4-methylbenzyl, 4-chlorobenzyl, 4-trifluorobenzyl, phenyl, 4-phenylbenzyl, 4-iodobenzyl, 3-methoxybenzyl, 4-cyanobenzyl, 4-bromobenzyl, 2-methoxybenzyl, 4-fluorobenzyl, 4-methoxybenzyl, 2-phenylethyl, 4-methoxycarbonylbenzyl, and (benzo-1,4-dioxane-6-yl)methyl.

In certain embodiments, R¹ is C₆₋₁₀ arylcarbonyl or C₁-C₁₀ alkylcarbonyl. In certain preferred embodiments, R¹ is acetyl or benzoyl.

In certain preferred embodiments, R¹ is C₆₋₁₀ arylsulfonyl. In a certain preferred embodiment, R¹ is phenylsulfonyl.

In certain embodiments, X is N and Y is CH.

In certain embodiments, E, F, G, and H are all absent and o is 1.

In certain embodiments, Ar¹ and Ar² are both phenyl. In certain preferred embodiments, R¹ is methyl or ethyl.

In certain embodiments, Ar¹ and Ar² are different. In certain preferred embodiments, Ar¹ is 4-chlorophenyl and Ar² is phenyl.

In certain preferred embodiments, R¹ is selected from methyl, ethyl, propyl, butyl, isopropyl, isobutyl, 2,2,2-trideuteroethyl, 2,2,2-trifluoroethyl, cyclopentyl, cyclohexyl, methylcarbonyl, (2,4-dimethoxyphenyl)methyl, 4-methylpiperazin-1-yl, 1-methylpiperidin-4-yl, 4-methylhomopiperazin-1-yl, —(CH₂)₂O(CH₂)₂COOH, —(CH₂)₂O(CH₂)₂OH, —(CH₂)₂O(CH₂)₂CONH₂, —CH₂CH₂OCH₂CH₂NH₂, —(CH₂CH₂O)₄CH₂CH₂NH₂, —(CH₂CH₂O)₄CH₂CH₂NHCOCH₃, and —(CH₂CH₂O)₄CH₂CH₂NHCOOt-Bu.

In certain embodiments, m and n are both 0 and o is 2. In certain preferred embodiments, Ar¹ is 4-chlorophenyl and Ar² is phenyl. In certain preferred embodiments, R¹ is methyl or ethyl.

In an embodiment, the invention provides a compound or a pharmaceutically acceptable salt of formula (I) and a pharmaceutically acceptable carrier.

The phrase “pharmaceutically acceptable salt” is intended to include nontoxic salts synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two. Generally, nonaqueous media such as ether, ethyl acetate, ethanol, isopropanol, or acetonitrile are preferred. Lists of suitable salts are found in Remington's Pharmaceutical Sciences, 18th ed., Mack Publishing Company, Easton, Pa., 1990, p. 1445, and Journal of Pharmaceutical Science, 66, 2-19 (1977).

Suitable bases include inorganic bases such as alkali and alkaline earth metal bases, e.g., those containing metallic cations such as sodium, potassium, magnesium, calcium and the like. Non-limiting examples of suitable bases include sodium hydroxide, potassium hydroxide, sodium carbonate, and potassium carbonate. Suitable acids include inorganic acids such as hydrochloric acid, hydrobromic acid, hydroiodic acid, sulfuric acid, phosphoric acid, and the like, and organic acids such as p-toluenesulfonic, methanesulfonic acid, benzenesulfonic acid, oxalic acid, p-bromophenylsulfonic acid, carbonic acid, succinic acid, citric acid, benzoic acid, acetic acid, maleic acid, tartaric acid, fatty acids, long chain fatty acids, and the like. Preferred pharmaceutically acceptable salts of inventive compounds having an acidic moiety include sodium and potassium salts. Preferred pharmaceutically acceptable salts of inventive compounds having a basic moiety (e.g., a dimethylaminoalkyl group) include hydrochloride and hydrobromide salts. The compounds of the present invention containing an acidic or basic moiety are useful in the form of the free base or acid or in the form of a pharmaceutically acceptable salt thereof

It should be recognized that the particular counterion forming a part of any salt of this invention is usually not of a critical nature, so long as the salt as a whole is pharmacologically acceptable and as long as the counterion does not contribute undesired qualities to the salt as a whole.

It is further understood that the above compounds and salts may form solvates, or exist in a substantially uncomplexed form, such as the anhydrous form. As used herein, the term “solvate” refers to a molecular complex wherein the solvent molecule, such as the crystallizing solvent, is incorporated into the crystal lattice. When the solvent incorporated in the solvate is water, the molecular complex is called a hydrate. Pharmaceutically acceptable solvates include hydrates, alcoholates such as methanolates and ethanolates, acetonitrilates and the like. These compounds can also exist in polymorphic forms.

In any of the above embodiments, the compound or salt of formula (I) can have at least one asymmetric carbon atom. When the compound or salt has at least one asymmetric carbon atom, the compound or salt can exist in the racemic form, in the form of its pure optical isomers, or in the form of a mixture wherein one isomer is enriched relative to the other. In particular, in accordance with the present invention, when the inventive compounds have a single asymmetric carbon atom, the inventive compounds may exist as racemates, i.e., as mixtures of equal amounts of optical isomers, i.e., equal amounts of two enantiomers, or in the form of a single enantiomer. As used herein, “single enantiomer” is intended to include a compound that comprises more than 50% of a single enantiomer (i.e., enantiomeric excess up to 100% pure enantiomer).

When the compound or salt has more than one chiral center, the compound or salt can therefore exist as a mixture of diastereomers or in the form of a single diastereomer. As used herein, “single diastereomer” is intended to mean a compound that comprises more than 50% of a single diastereomer (i.e., diastereomeric excess to 100% pure diastereomer).

The present invention further provides a pharmaceutical composition comprising a compound as described above and a pharmaceutically acceptable carrier. The present invention provides a pharmaceutical composition comprising a pharmaceutically acceptable carrier and an effective amount, e.g., a therapeutically effective amount, including a prophylactically effective amount, of one or more of the aforesaid compounds, or salts thereof, of the present invention.

The pharmaceutically acceptable carrier can be any of those conventionally used and is limited only by chemico-physical considerations, such as solubility and lack of reactivity with the compound, and by the route of administration. It will be appreciated by one of skill in the art that, in addition to the following described pharmaceutical compositions; the compounds of the present invention can be formulated as inclusion complexes, such as cyclodextrin inclusion complexes, or liposomes.

The pharmaceutically acceptable carriers described herein, for example, vehicles, adjuvants, excipients, or diluents, are well known to those who are skilled in the art and are readily available to the public. It is preferred that the pharmaceutically acceptable carrier be one which is chemically inert to the active compounds and one which has no detrimental side effects or toxicity under the conditions of use.

The choice of carrier will be determined in part by the particular active agent, as well as by the particular method used to administer the composition. Accordingly, there is a wide variety of suitable formulations of the pharmaceutical composition of the present invention. The following formulations for oral, aerosol, parenteral, subcutaneous, intravenous, intraarterial, intramuscular, interperitoneal, intrathecal, rectal, and vaginal administration are merely exemplary and are in no way limiting.

Formulations suitable for oral administration can consist of (a) liquid solutions, such as an effective amount of the compound dissolved in diluents, such as water, saline, or orange juice; (b) capsules, sachets, tablets, lozenges, and troches, each containing a predetermined amount of the active ingredient, as solids or granules; (c) powders; (d) suspensions in an appropriate liquid; and (e) suitable emulsions. Liquid formulations may include diluents, such as water and alcohols, for example, ethanol, benzyl alcohol, and the polyethylene alcohols, either with or without the addition of a pharmaceutically acceptable surfactant, suspending agent, or emulsifying agent. Capsule forms can be of the ordinary hard- or soft-shelled gelatin type containing, for example, surfactants, lubricants, and inert fillers, such as lactose, sucrose, calcium phosphate, and cornstarch. Tablet forms can include one or more of lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and pharmacologically compatible carriers. Lozenge forms can comprise the active ingredient in a flavor, usually sucrose and acacia or tragacanth, as well as pastilles comprising the active ingredient in an inert base, such as gelatin and glycerin, or sucrose and acacia, emulsions, gels, and the like containing, in addition to the active ingredient, such carriers as are known in the art.

The compounds of the present invention, alone or in combination with other suitable components, can be made into aerosol formulations to be administered via inhalation. These aerosol formulations can be placed into pressurized acceptable propellants, such as dichlorodifluoromethane, propane, nitrogen, and the like. They also may be formulated as pharmaceuticals for non-pressured preparations, such as in a nebulizer or an atomizer.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The compound can be administered in a physiologically acceptable diluent in a pharmaceutical carrier, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol, glycerol ketals, such as 2,2-dimethyl-1,3-dioxolane-4-methanol, ethers, such as poly(ethyleneglycol) 400, an oil, a fatty acid, a fatty acid ester or glyceride, or an acetylated fatty acid glyceride with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

Oils, which can be used in parenteral formulations include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters. Suitable soaps for use in parenteral formulations include fatty alkali metal, ammonium, and triethanolamine salts, and suitable detergents include (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylene-polypropylene copolymers, (d) amphoteric detergents such as, for example, alkyl-beta-aminopropionates, and 2-alkyl-imidazoline quaternary ammonium salts, and (3) mixtures thereof.

The parenteral formulations will typically contain from about 0.5 to about 25% by weight of the active ingredient in solution. Suitable preservatives and buffers can be used in such formulations. In order to minimize or eliminate irritation at the site of injection, such compositions may contain one or more nonionic surfactants having a hydrophile-lipophile balance (HLB) of from about 12 to about 17. The quantity of surfactant in such formulations ranges from about 5 to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol. The parenteral formulations can be presented in unit-dose or multi-dose sealed containers, such as ampoules and vials, and can be stored in a freeze-dried (lyophilized) condition requiring only the addition of the sterile liquid carrier, for example, water, for injections, immediately prior to use. Extemporaneous injection solutions and suspensions can be prepared from sterile powders, granules, and tablets of the kind previously described.

The compounds of the present invention may be made into injectable formulations. The requirements for effective pharmaceutical carriers for injectable compositions are well known to those of ordinary skill in the art. See Pharmaceutics and Pharmacy Practice, J. B. Lippincott Co., Philadelphia, Pa., Banker and Chalmers, eds., pages 238-250 (1982), and ASHP Handbook on Injectable Drugs, Toissel, 4th ed., pages 622-630 (1986).

Additionally, the compounds of the present invention may be made into suppositories by mixing with a variety of bases, such as emulsifying bases or water-soluble bases. Formulations suitable for vaginal administration may be presented as pessaries, tampons, creams, gels, pastes, foams, or spray formulas containing, in addition to the active ingredient, such carriers as are known in the art to be appropriate.

In an embodiment, the invention provides a method of treating or preventing a viral infection in a mammal in need thereof comprising administering to the mammal an effective amount of a compound of formula (I):

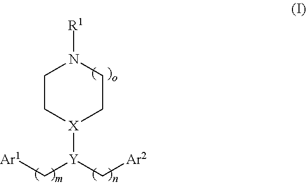

wherein R¹ is selected from hydrogen, C₁-C₁₀ alkyl, C₃-C₁₀ cycloalkyl, C₃-C₁₀ cycloalkyl C₁-C₁₀ alkyl, C₆-C₁₀ aryl, C₆-C₁₀ aryl C₁-C₁₀ alkyl, C₆-C₁₀ aryl C₃-C₁₀ cycloalkyl, heteroaryl, heterocyclyl, C₆₋₁₀ arylsulfonyl, C₆₋₁₀ arylcarbonyl, C₁-C₁₀ alkylcarbonyl, —(CH₂)ₓA(CH₂)_{y}B, wherein the alkyl, aryl, or heteroaryl part of R¹ is optionally substituted with one or more substituents selected from deuterium, halo, C₁-C₁₀ alkyl, C₆-C₁₀ aryl, trifluoromethyl, C₁-C₁₀ alkoxy, cyano, alkylenedioxy, C₁-C₁₀ alkylcarbonyl, and C₁-C₁₀ alkoxycarbonyl,

Ar¹ and Ar² are the same or different and are independently selected from C₆-C₁₀ aryl, heteroaryl, and heterocyclyl, wherein the aryl, heteroaryl, and heterocyclyl are optionally substituted with one or more substituents selected from halo, C₁-C₁₀ alkyl, C₆-C₁₀ aryl, trifluoromethyl, C₁-C₁₀ alkoxy, C₁-C₁₀ alkylcarbonyl, and C₁-C₁₀ alkoxycarbonyl,

A is O, S, or N,

x and y are independently 1-4, inclusive,

B is selected from OR⁴, COOR⁵, and CONR⁶R⁷,

wherein R⁴, R⁵, R⁶, and R⁷ are independently selected from hydrogen, C₁-C₁₀ alkyl, C₃-C₁₀ cycloalkyl, and C₆-C₁₀ aryl,

m and n are independently 0, 1, 2, 3, or 4,

o is 0, 1, or 2,

X and Y are independently CH or N,

or pharmaceutically acceptable salts, stereoisomers, and mixtures comprising stereoisomers thereof.

In certain embodiments, X is CH and Y is N.

In certain embodiments, o is 1. In certain embodiments, m is 2. In certain embodiments, n is 1.

In certain embodiments, Ar¹ and Ar² are both phenyl.

In certain embodiments, R¹ is selected from C₁-C₁₀ alkyl, C₃-C₁₀ cycloalkyl, and C₃-C₁₀ cycloalkyl C₁-C₁₀ alkyl.

In certain preferred embodiments, R¹ is selected from hydrogen, cyclopentyl, sec-butyl, isopropyl, cyclohexyl, n-propyl, n-butyl, benzoyl, methyl, ethyl, trideuteromethyl, 2,2,2-trideuteroethyl, 2,2,2-trifluoroethyl, phenylsulfonyl, and benzyl.

In certain embodiments, R¹ is selected from C₆-C₁₀ aryl and C₆-C₁₀ aryl C₁-C₁₀ alkyl, wherein the aryl is optionally substituted with one or more substituents selected from halo, cyano, alkylenedioxy, C₁-C₁₀ alkyl, C₆-C₁₀ aryl, trifluoromethyl, C₁-C₁₀ alkoxy, cyano, alkylenedioxy, C₁-C₁₀ alkylcarbonyl, and C₁-C₁₀ alkoxycarbonyl.

In certain preferred embodiments, R¹ is selected from 4-methylbenzyl, 4-chlorobenzyl, 4-trifluorobenzyl, phenyl, 4-phenylbenzyl, 4-iodobenzyl, 3-methoxybenzyl, 4-cyanobenzyl, 4-bromobenzyl, 2-methoxybenzyl, 4-fluorobenzyl, 4-methoxybenzyl, 2-phenylethyl, 4-methoxycarbonylbenzyl, and (benzo-1,4-dioxane-6-yl)methyl.

In certain embodiments, R¹ is C₆₋₁₀ arylcarbonyl or C₁-C₁₀ alkylcarbonyl. In certain preferred embodiments, R¹ is acetyl or benzoyl.

In certain preferred embodiments, R¹ is C₆₋₁₀ arylsulfonyl. In a certain preferred embodiment, R¹ is phenylsulfonyl.

In certain embodiments, X is N and Y is CH.

In certain embodiments, m and n are both 0 and o is 1.

In certain embodiments, Ar¹ and Ar² are both phenyl. In certain preferred embodiments, R¹ is methyl or ethyl.

In certain embodiments, Ar¹ and Ar² are different. In certain preferred embodiments, Ar¹ is 4-chlorophenyl and Ar² is phenyl.

In certain preferred embodiments, R¹ is selected from methyl, ethyl, propyl, butyl, isopropyl, isobutyl, 2,2,2-trideuteromethyl, 2,2,2-trifluoroethyl, cyclopentyl, cyclohexyl, methylcarbonyl, (2,4-dimethoxyphenyl)methyl, 4-methylpiperazin-1-yl, 1-methylpiperidin-4-yl, 4-methylhomopiperazin-1-yl, —(CH₂)₂O(CH₂)₂COOH, —(CH₂)₂O(CH₂)₂OH, and —(CH₂)₂O(CH₂)₂CONH₂.

In certain embodiments, m and n are both 0 and o is 2. In certain preferred embodiments, Ar¹ is 4-chlorophenyl and Ar² is phenyl. In certain preferred embodiments, R¹ is methyl or ethyl.

In a preferred embodiment, the invention provides a method for treating or preventing hepatitis C.

In an embodiment, the inventive method further comprises administering to the mammal an effective amount of an anti-hepatitis C compound other than the compound of formula (I). Non-limiting examples of suitable anti-hepatitis C compounds include ribavirin, interferon-α, telaprevir, cyclosporin A, Asunaprevir (BMS-650032), Boceprevir, GS-9451, GS-9256, ABT-450, Danoprevir (RG7227), Faldaprevir (BI 201335), IDX320, MK-5172, Simeprevir (TMC435), Sovaprevir (ACH-1625), ABT-267, ACH-3102, BMS-791325, Daclatasvir (BMS-790052), GSK2336805, IDX719, JNJ-47910382, Ledipasvir (GS-5885), MK-8742, PPI-461, PPI-668, ABT-333, ALS-002200, BI 207127, IDX184, INX-08189, Mericitabine (RO5024048), PPI-383, PSI-352938, Setrobuvir (ANA-598), Sofosbuvir (PSI-7977 or GS-7977), Tegobuvir (GS-9190), TMC647055, Filibuvir (PF-00868554), GS-9669, GSK2878175, VX-135, VX-222, Algeron (Cepeginterferon Alfa-2b), BIP 48 (Peginterferon alfa 2b 48 kDA), Pegylated interferon alfa 2b, Pegylated interferon lambda (BMS-914143), Pegylated-P-Interferon-alpha-2b (P1101), and Alisporivir (DEB025).

In an embodiment, the invention provides a method for synergistically enhancing the antiviral effect of an anti-hepatitis C compound in a mammal undergoing treatment with the anti-hepatitis C compound, which method comprises administering to the mammal a compound of the formula (I). The compound of formula (I) can be as described herein in connection with the method for treating or preventing hepatitis C.

In other embodiments, the inventive method is suitable for the treatment of a virus other than hepatitis C virus. For example, the inventive method is suitable for the treatment of a virus selected from Flaviviridae family of viruses such as West Nile virus, yellow fever virus, Japanese encephalitis virus, or dengue virus, and other families of viruses such as but not limiting to rhinovirus, polio virus, hepatitis A virus, hepatitis B virus, and the like.

“Treatment” refers to a therapeutic intervention that ameliorates a sign or symptom of a disease or pathological condition after it has begun to develop. As used herein, the term “ameliorating,” with reference to a disease or pathological condition, refers to any observable beneficial effect of the treatment. The beneficial effect can be evidenced, for example, by a delayed onset of clinical symptoms of the disease in a susceptible subject, a reduction in severity of some or all clinical symptoms of the disease, a slower progression of the disease, an improvement in the overall health or well-being of the subject, or by other parameters well known in the art that are specific to the particular disease. Treatment of hepatitis C can be evidenced, for example, by a reduction in viral burden, a reduction in clinical symptoms resulting from the viral infection, or other parameters well known in the art that are specific to the viral infection, for example the hepatitis C infection. Treatment of cancer can be evidenced, for example, by a reduction in tumor size, a reduction in tumor burden, a reduction in clinical symptoms resulting from the cancer, or other parameters well known in the art that are specific to the cancer. The phrase “treating a disease” refers to inhibiting the full development of a disease or condition, for example, in a subject who is at risk for a disease such as cancer, particularly a metastatic cancer. As used herein, the term “preventing,” with reference to a disease or pathological condition, refers to blocking the appearance of a disease or a symptom associated with the disease, for example, the presence of a viral load, in an asymptomatic subject at risk of developing the disease, for example, by way of exposure to a virus.

By the term “coadminister” is meant that each of the at least two compounds be administered during a time frame wherein the respective periods of biological activity overlap. Thus, the term includes sequential as well as coextensive administration of two or more drug compounds. The compounds can be administered simultaneously, separately (chronologically staggered), cyclically, or sequentially and in any order, e.g., before or after.

The doses of the compound of formula (I) and/or the anti-hepatitis C compound administered to a mammal, particularly, a human, in accordance with the present invention should be sufficient to effect the desired response. Such responses include reversal or prevention of the adverse effects of the disease for which treatment is desired or to elicit the desired benefit. One skilled in the art will recognize that dosage will depend upon a variety of factors, including the age, condition, and body weight of the human, as well as the source, particular type of the disease, and extent of the disease in the human. The size of the doses will also be determined by the routes, timing and frequency of administration as well as the existence, nature, and extent of any adverse side-effects that might accompany the administration of a particular compound and the desired physiological effect. It will be appreciated by one of skill in the art that various conditions or disease states may require prolonged treatment involving multiple administrations.

Suitable doses and dosage regimens can be determined by conventional range-finding techniques known to those of ordinary skill in the art. Generally, treatment is initiated with smaller dosages that are less than the optimum dose of the compounds. Thereafter, the dosage is increased by small increments until the optimum effect under the circumstances is reached. The present inventive method typically will involve the administration of about 0.1 to about 300 mg of one or more of the compounds described above per kg body weight of the animal or mammal.

The therapeutically effective amount of the compound or compounds administered can vary depending upon the desired effects and the factors noted above. Typically, dosages will be between 0.01 mg/kg and 250 mg/kg of the subject's body weight, and more typically between about 0.05 mg/kg and 100 mg/kg, such as from about 0.2 to about 80 mg/kg, from about 5 to about 40 mg/kg or from about 10 to about 30 mg/kg of the subject's body weight. Thus, unit dosage forms can be formulated based upon the suitable ranges recited above and the subject's body weight. The term “unit dosage form” as used herein refers to a physically discrete unit of therapeutic agent appropriate for the subject to be treated.

Alternatively, dosages are calculated based on body surface area and from about 1 mg/m² to about 200 mg/m², such as from about 5 mg/m² to about 100 mg/m² will be administered to the subject per day. In particular embodiments, administration of the therapeutically effective amount of the compound or compounds involves administering to the subject from about 5 mg/m² to about 50 mg/m², such as from about 10 mg/m² to about 40 mg/m² per day. It is currently believed that a single dosage of the compounds is suitable, however a therapeutically effective dosage can be supplied over an extended period of time or in multiple doses per day. Thus, unit dosage forms also can be calculated using a subject's body surface area based on the suitable ranges recited above and the desired dosing schedule.

In accordance with an embodiment, the invention provides a method of treating cancer in a mammal in need thereof, comprising administering to the animal a compound of formula (I) or pharmaceutically acceptable salts, stereoisomers, and mixtures comprising stereoisomers thereof. In accordance with these embodiments, the compound or salts, stereoisomers, and mixtures comprising stereoisomers thereof, of the invention is administered to the mammal by itself, i.e., without co-administration of an anticancer agent, radiation, or biotherapeutic agent. In some embodiments, the compound or salts, stereoisomers, and mixtures comprising stereoisomers thereof of the invention can be administered concomitantly with radiation and/or biotherapeutic agent.

The cancer can be any suitable cancer. For example, the cancer may be adrenocortical carcinoma, AIDS-related lymphoma, AIDS-related malignancies, anal cancer, cerebellar astrocytoma, extrahepatic bile duct cancer, bladder cancer, osteosarcoma/malignant fibrous histiocytoma, brain stem glioma, ependymoma, visual pathway and hypothalamic gliomas, breast cancer, bronchial adenomas/carcinoids, carcinoid tumors, gastrointestinal carcinoid tumors, carcinoma, adrenocortical, islet cell carcinoma, primary central nervous system lymphoma, cerebellar astrocytoma, cervical cancer, chronic lymphocytic leukemia, chronic myelogenous leukemia, clear cell sarcoma of tendon sheaths, colon cancer, colorectal cancer, cutaneous t-cell lymphoma, endometrial cancer, ependymoma, esophageal cancer, Ewing's sarcoma/family of tumors, extracranial germ cell tumors, extragonadal germ cell tumors, extrahepatic bile duct cancer, eye cancers, including intraocular melanoma, and retinoblastoma, gallbladder cancer, gastrointestinal carcinoid tumor, ovarian germ cell tumor, gestational trophoblastic tumor, hairy cell leukemia, head and neck cancer, Hodgkin's disease, hypopharyngeal cancer, hypothalamic and visual pathway glioma, intraocular melanoma, Kaposi's sarcoma, laryngeal cancer, acute lymphoblastic leukemia, acute myeloid leukemia, liver cancer, non-small cell lung cancer, small cell lung cancer, non-Hodgkin's lymphoma, Waldenstrom's macroglobulinemia, malignant mesothelioma, malignant thymoma, medulloblastoma, melanoma, intraocular melanoma, merkel cell carcinoma, metastatic squamous neck cancer with occult primary, multiple endocrine neoplasia syndrome, multiple myeloma/plasma cell neoplasm, mycosis fungoides, myelodysplastic syndrome, chronic myelogenous leukemia, myeloid leukemia, multiple myeloma, myeloproliferative disorders, nasal cavity and paranasal sinus cancer, nasopharyngeal cancer, neuroblastoma, oral cancer, oral cavity and lip cancer, oropharyngeal cancer, osteosarcoma/malignant fibrous histiocytoma of bone, ovarian cancer, ovarian low malignant potential tumor, pancreatic cancer, paranasal sinus and nasal cavity cancer, parathyroid cancer, penile cancer, pheochromocytoma, pituitary tumor, pleuropulmonary blastoma, prostate cancer, rectal cancer, renal cell (kidney) cancer, transitional cell cancer (e.g. renal pelvis and ureter), retinoblastoma, rhabdomyosarcoma, salivary gland cancer, malignant fibrous histiocytoma of bone, soft tissue sarcoma, sezary syndrome, skin cancer, small intestine cancer, stomach (gastric) cancer, supratentorial primitive neuroectodermal and pineal tumors, cutaneous t-cell lymphoma, testicular cancer, malignant thymoma, thyroid cancer, gestational trophoblastic tumor, urethral cancer, uterine sarcoma, vaginal cancer, vulvar cancer, and Wilms' tumor. In a preferred embodiment, the cancer is a non-small cell lung cancer.

In any of the embodiments of the invention, the cancer can be any cancer in any organ, for example, a cancer is selected from the group consisting of glioma, thyroid carcinoma, breast carcinoma, small-cell lung carcinoma, non-small-cell carcinoma, gastric carcinoma, colon carcinoma, gastrointestinal stromal carcinoma, pancreatic carcinoma, bile duct carcinoma, CNS carcinoma, ovarian carcinoma, endometrial carcinoma, prostate carcinoma, renal carcinoma, anaplastic large-cell lymphoma, leukemia, multiple myeloma, mesothelioma, and melanoma, and combinations thereof

In an embodiment, the invention provides a pharmaceutical pack or kit comprising a compound of formula (I) and an anti-hepatitis C compound other than a compound of formula (I). The pharmaceutical pack or kit comprising one or more containers filled with a compound of formula (I) and an anti-hepatitis C compound other than a compound of formula (I). Optionally associated with such container(s) can be a notice in the form prescribed by a governmental agency regulating the manufacture, use or sale of pharmaceuticals or biological products, which notice reflects approval by the agency of manufacture, use or sale for human administration.

The following examples further illustrate the invention but, of course, should not be construed as in any way limiting its scope.

### Example 1

This example demonstrates a method of synthesis of compounds in accordance with an embodiment of the invention.

General Chemistry Methods. All air or moisture sensitive reactions were performed under positive pressure of nitrogen with oven-dried glassware. Anhydrous solvents such as dichloromethane, N,N-dimethylformamide (DMF), acetonitrile, methanol and triethylamine were purchased from Sigma-Aldrich (St. Louis, Mo.). Preparative purification was performed on a Waters semi-preparative HPLC system (Waters Corp., Milford, Mass.). The column used was a Phenomenex Luna C₁₈ (5 micron, 30×75 mm; Phenomenex, Inc., Torrance, Calif.) at a flow rate of 45.0 mL/min. The mobile phase consisted of acetonitrile and water (each containing 0.1% trifluoroacetic acid). A gradient of 10% to 50% acetonitrile over 8 min was used during the purification. Fraction collection was triggered by UV detection at 220 nM. Chromotographic analysis was performed on an Agilent LC/MS (Agilent Technologies, Santa Clara, Calif.). Method 1: A 7-min gradient of 4% to 100% acetonitrile (containing 0.025% trifluoroacetic acid) in water (containing 0.05% trifluoroacetic acid) was used with an 8-min run time at a flow rate of 1.0 mL/min. Method 2: A 3-min gradient of 4% to 100% acetonitrile (containing 0.025% trifluoroacetic acid) in water (containing 0.05% trifluoroacetic acid) was used with a 4.5-min run time at a flow rate of 1.0 mL/min. A Phenomenex Luna C₁₈ column (3 micron, 3×75 mm) was used at a temperature of 50° C. Purity determination was performed using an Agilent diode array detector for both Method 1 and Method 2. Mass determination was performed using an Agilent 6130 mass spectrometer with electrospray ionization in the positive mode. ¹H NMR spectra were recorded on Varian 400 MHz spectrometers (Agilent Technologies, Santa Clara, Calif.). Chemical shifts are reported in ppm with undeuterated solvent (DMSO at 2.49 ppm) as internal standard for DMSO-d₆ solutions. All of the analogs tested in the biological assays have a purity of greater than 95% based on both analytical methods. High resolution mass spectrometry was recorded on Agilent 6210 Time-of-Flight (TOF) LC/MS system. Confirmation of molecular formula was accomplished using electrospray ionization in the positive mode with the Agilent Masshunter software (Version B.02).

General Protocol A. A solution of amine (0.157 mmol) and aldehyde or ketone (0.314 mmol, 2.0 equiv.) in ethanol (2.00 mL) was treated at room temperature with titanium (IV) isopropoxide (0.092 mL, 0.314 mmol, 2.0 equiv.). The reaction mixture was stirred at room temperature for 10 min and treated with NaCNBH₄ (49.3 mg, 0.785 mmol, 5.0 equiv.). The resulting mixture was stirred at room temperature for 1-8 h and quenched at room temperature with 1 N NaOH. The mixture was dried by blowing air, re-dissolved in DMSO, filtered and purified by preparative HPLC to give the final product.

General Protocol B. A solution of amine (0.105 mmol) in MeOH (1.00 mL) was treated at room temperature with aldehyde (0.525 mmol to 1.05 mmol, 5.0 to 10.0 equiv.), NaCNBH₄ (19.7 mg, 0.315 mmol, 3.0 equiv.) and acetic acid (0.018 mL, 0.315 mmol, 3.0 mmol). The reaction mixture was stirred at room temperature for 1-8 h and quenched with 1 N NaOH solution. The mixture was dried by blowing air, re-dissolved in DMSO, filtered and purified by HPLC.

### N-Benzyl-1-(2,4-dimethoxybenzyl)-N-phenethylpiperidin-4-amine (NCGC00345021-03)

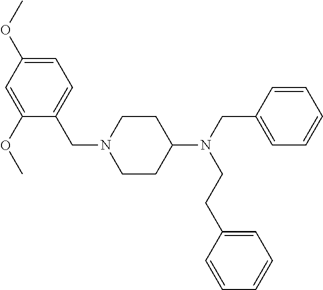 [SMILES: COc1ccc(CN2CCC(N(CCc3ccccc3)Cc3ccccc3)CC2)c(OC)c1]

The title compound was prepared according to General Protocol B as a TFA salt. LCMS t₁ (Method 1)=3.990 min, m/z 445.2 [M+H⁺].

### N-Benzyl-N-phenethylpiperidin-4-amine (NCGC00346843-01, XJB14-021)

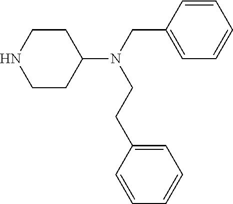 [SMILES: c1ccc(CCN(Cc2ccccc2)C2CCNCC2)cc1]

The title compound as HCl salt was purchased from ChemBridge, catalog #6766468. The sample was converted to its TFA salt using reverse phase HPLC. LCMS t₁ (Method 1)=3.276 min, m/z 295.1 [M+H⁺].

### (4-(Benzyl(phenethyl)amino)piperidin-1-yl)(phenyl)methanone (NCGC00346844-01, XJB14-022)

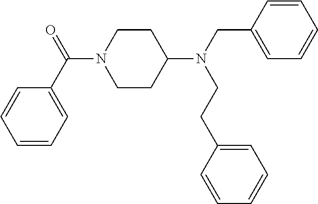 [SMILES: O=C(c1ccccc1)N1CCC(N(CCc2ccccc2)Cc2ccccc2)CC1]

A solution of N-benzyl-N-phenethylpiperidin-4-amine (30.0 mg, 0.102 mmol) in dichloromethane (2.00 mL) was treated at room temperature with triethylamine (0.071 mL, 0.509 mmol) followed by benzoyl chloride (28.6 mg, 0.204 mmol). The reaction mixture was stirred at room temperature overnight. The mixture was dried by blowing air, re-dissolved in DMSO, filtered and purified by HPLC to give the title compound as a TFA salt. ¹H NMR (400 MHz, DMSO-d₆) δ 9.49 (s, 1H), 7.65-7.58 (m, 2H), 7.57-7.13 (m, 13H), 4.59 (dd, J=3.79, 13.32 Hz, 1H), 4.37 (dd, J=6.68, 13.36 Hz, 1H), 3.79-3.61 (m, 3H), 3.20 (td, J=6.00, 12.11, 12.79 Hz, 2H), 2.99 (td, J=5.10, 12.65 Hz, 2H), 2.82-2.73 (m, 2H), 1.93-1.79 (m, 4H); LCMS t₁ (Method 1)=4.375 min, m/z 399.2 [M+H⁺].

### N-Benzyl-1-methyl-N-phenethylpiperidin-4-amine (NCGC00346846-01, XJB14-026)

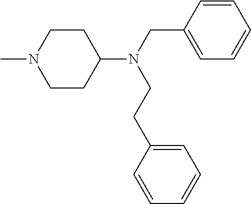 [SMILES: CN1CCC(N(CCc2ccccc2)Cc2ccccc2)CC1]

The title compound was prepared according to General Protocol B as a TFA salt. LCMS t₁ (Method 1)=3.422 min, m/z 309.2 [M+H⁺].

### N-Benzyl-1-ethyl-N-phenethylpiperidin-4-amine (NCGC00346847-01, XJB14-027, XJB015-074)

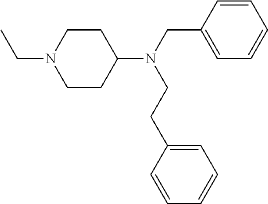 [SMILES: CCN1CCC(N(CCc2ccccc2)Cc2ccccc2)CC1]

The title compound was prepared according to General Protocol B as a TFA salt. ¹H NMR (400 MHz, DMSO-d₆) δ 7.33-7.09 (m, 10H), 3.71 (s, 2H), 3.50-3.41 (m, 2H), 3.02 (q, J=7.26 Hz, 2H), 2.82 (d, J=12.01 Hz, 3H), 2.67 (s, 4H), 1.87 (d, J=12.29 Hz, 2H), 1.70 (q, J=13.07 Hz, 2H), 1.19 (h, J=11.19, 12.50 Hz, 3H); LCMS t₁ (Method 1)=3.345 min, m/z 323.2 [M+H⁺].

### N-Benzyl-N-phenethyl-1-(phenylsulfonyl)piperidin-4-amine (NCGC00346849-01, XJB14-035)

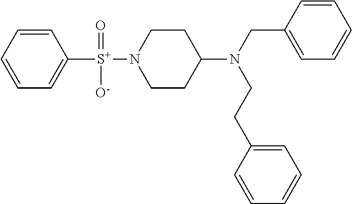 [SMILES: O=[S+]([O-])(c1ccccc1)N1CCC(N(CCc2ccccc2)Cc2ccccc2)CC1]

A solution of N-benzyl-N-phenethylpiperidin-4-amine (30.0 mg, 0.102 mmol) in dichloromethane (2.00 mL) was treated at room temperature with triethylamine (0.071 mL, 0.509 mmol) followed by benzenesulfonyl chloride (36.0 mg, 0.204 mmol). The reaction mixture was stirred at room temperature overnight. The mixture was dried by blowing air, re-dissolved in DMSO, filtered and purified by HPLC to give the title compound as a TFA salt. TFA salt. LCMS t₁ (Method 1)=4.648 min, m/z 435.2 [M+H⁺].

### N,1-dibenzyl-N-phenethylpiperidin-4-amine (NCGC00346850-01, XJB14-036)

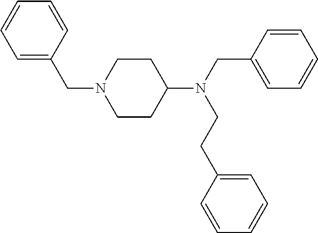 [SMILES: c1ccc(CCN(Cc2ccccc2)C2CCN(Cc3ccccc3)CC2)cc1]

The title compound was prepared according to General Protocol A as a TFA salt. LCMS t₁ (Method 1)=3.701 min, m/z 385.2 [M+H⁺].

### N-(4-(tert-Butoxy)phenyl)-1-methyl-N-phenylpiperidin-4-amine (NCGC00346851-01, XJB14-042)

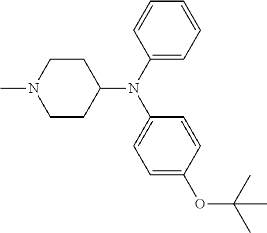 [SMILES: CN1CCC(N(c2ccccc2)c2ccc(OC(C)(C)C)cc2)CC1]

A mixture of N-(4-chlorophenyl)-1-methylpiperidin-4-amine (30.0 mg, 0.133 mmol), iodobenzene (0.030 mL, 0.267 mmol), Pd(OAc)₂ (3.00 mg, 0.013 mmol), BINAP (9.14 mg, 0.015 mmol) in toluene (0.200 mL) was treated at room temperature with potassium tert-butoxide (0.167 mL, 1.0 M solution in THF, 0.167 mmol). The reaction mixture was stirred at 110° C. for 4 h. The mixture was cooled to room temperature, dried by blowing air, re-dissolved in DMSO, filtered and purified by HPLC to give the title compound as a TFA salt. TFA salt. LCMS t₁ (Method 1)=4.656 min, m/z 339.1 [M+H⁺].

### N-Benzyl-1-cyclopentyl-N-phenethylpiperidin-4-amine (NCGC00347035-01, XJB14-068)

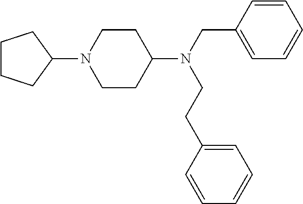 [SMILES: c1ccc(CCN(Cc2ccccc2)C2CCN(C3CCCC3)CC2)cc1]

The title compound was prepared according to General Protocol A as a TFA salt. LCMS t₁ (Method 1)=3.476 min, m/z 363.2 [M+H⁺].

### N-Benzyl-1-(4-methylbenzyl)-N-phenethylpiperidin-4-amine (NCGC00347037-01, XJB14-072)

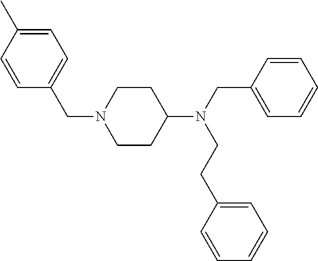 [SMILES: Cc1ccc(CN2CCC(N(CCc3ccccc3)Cc3ccccc3)CC2)cc1]

The title compound was prepared according to General Protocol A as a TFA salt. LCMS t₁ (Method 1)=3.864 min, m/z 399.3 [M+H⁺].

### N-Benzyl-1-(4-chlorobenzyl)-N-phenethylpiperidin-4-amine (NCGC00347038-01, XJB14-073)

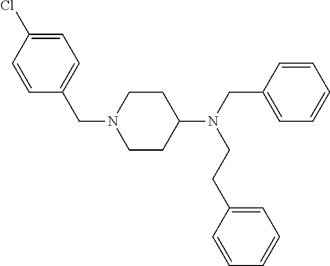 [SMILES: Clc1ccc(CN2CCC(N(CCc3ccccc3)Cc3ccccc3)CC2)cc1]

The title compound was prepared according to General Protocol A as a TFA salt. LCMS t₁ (Method 1)=3.933 min, m/z 419.2 [M+H⁺].

### N-Benzyl-1-isobutyl-N-phenethylpiperidin-4-amine (NCGC00347041-01, XJB14-086)

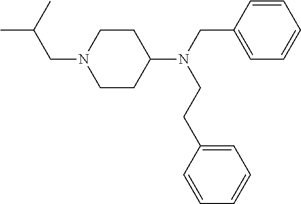 [SMILES: CC(C)CN1CCC(N(CCc2ccccc2)Cc2ccccc2)CC1]

The title compound was prepared according to General Protocol B as a TFA salt. LCMS t₁ (Method 1)=3.496 min, m/z 351.3 [M+H⁺].

### N-Benzyl-1-isopropyl-N-phenethylpiperidin-4-amine (NCGC00347043-01, XJB14-066)

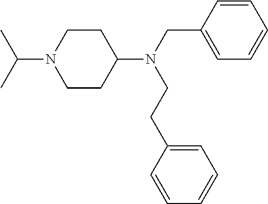 [SMILES: CC(C)N1CCC(N(CCc2ccccc2)Cc2ccccc2)CC1]

A solution of N-benzyl-N-phenethylpiperidin-4-amine (30.0 mg, 0.102 mmol) and propan-2-one (59.2 mg, 1.019 mmol) in ethanol (2.00 mL) was treated at room temperature with Ts-OH (2.91 mg, 0.015 mmol). The reaction mixture was stirred at room temperature for 10 min, then NaCNBH₄ (64.0 mg, 1.019 mmol) was added. The reaction mixture was stirred at room temperature over night. The mixture was cooled to room temperature, dried by blowing air, re-dissolved in DMSO, filtered and purified by HPLC to give the title compound as a TFA salt. LCMS t₁ (Method 1)=3.340 min, m/z 337.2 [M+H⁺].

### N-Benzyl-N-phenethyl-1-(4-(trifluoromethyl)benzyl)piperidin-4-amine (NCGC00347045-01, XJB14-063)

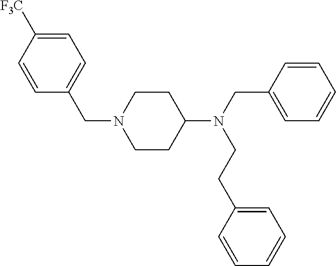 [SMILES: FC(F)(F)c1ccc(CN2CCC(N(CCc3ccccc3)Cc3ccccc3)CC2)cc1]

The title compound was prepared according to General Protocol A as a TFA salt.

### N-Benzyl-1-cyclohexyl-N-phenethylpiperidin-4-amine (NCGC00347046-01, XJB14-049)

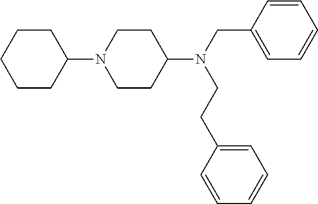 [SMILES: c1ccc(CCN(Cc2ccccc2)C2CCN(C3CCCCC3)CC2)cc1]

The title compound was prepared according to General Protocol A as a TFA salt. LCMS t₁ (Method 1)=3.647 min, m/z 377.2 [M+H⁺].

### N-Benzyl-N-phenethyl-1-phenylpiperidin-4-amine (NCGC00347047-01, XJB14-051)

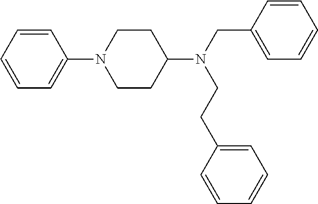 [SMILES: c1ccc(CCN(Cc2ccccc2)C2CCN(c3ccccc3)CC2)cc1]

A mixture of N-benzyl-N-phenethylpiperidin-4-amine (30.0 mg, 0.102 mmol), phenylboronic acid (18.6 mg, 0.153 mmol), DBU (0.031 mL, 0.204 mmol), and copper (II) acetate (37.0 mg, 0.204 mmol) in DMSO (2.00 mL) was heated in ON at 100° C. for 1 h. The mixture was cooled to room temperature and filtered through a cartridge of Tiol to get rid of copper, and purified by HPLC to give the title compound as a TFA salt. ¹H NMR (400 MHz, DMSO-d₆) δ 9.49 (s, 1H), 7.66-7.59 (m, 2H), 7.51 (dd, J=2.13, 4.99 Hz, 3H), 7.37-7.12 (m, 7H), 6.98 (d, J=8.15 Hz, 2H), 6.79 (t, J=7.26 Hz, 1H), 4.63 (dd, J=3.55, 13.73 Hz, 1H), 4.35 (dd, J=7.16, 13.25 Hz, 1H), 3.88 (d, J=11.82 Hz, 2H), 3.72-3.54 (m, 1H), 3.29-3.17 (m, 1H), 3.01 (td, J=5.69, 12.28 Hz, 1H), 2.85-2.68 (m, 4H), 2.22-2.17 (m, 2H), 1.94 (td, J=6.27, 11.29, 11.94 Hz, 2H); LCMS t₁ (Method 1)=4.733 min, m/z 371.2 [M+H⁺].

### 1-(4-(Benzyl(phenethyl)amino)piperidin-1-yl)ethanone (NCGC00347048-01, XJB14-070)

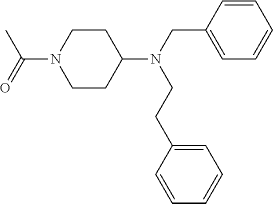 [SMILES: CC(=O)N1CCC(N(CCc2ccccc2)Cc2ccccc2)CC1]

A solution of N-benzyl-N-phenethylpiperidin-4-amine (30.0 mg, 0.102 mmol) in dichloromethane (2.00 mL) was treated at room temperature with acetyl chloride (16.0 mg, 0.204 mmol) and triethylamine (0.043 mL, 0.306 mmol). The reaction mixture was stirred at room temperature for 1 h. The mixture was dried by blowing air, re-dissolved in DMSO, filtered and purified by HPLC to give the title compound as a TFA salt. ¹H NMR (400 MHz, DMSO-d₆) δ 9.48 (s, 1H), 7.65-7.57 (m, 2H), 7.51 (dd, J=2.05, 5.03 Hz, 3H), 7.36-7.20 (m, 3H), 7.20-7.13 (m, 2H), 4.56 (dt, J=4.28, 13.66 Hz, 2H), 4.34 (dt, J=5.74, 12.59 Hz, 1H), 3.96 (d, J=13.35 Hz, 1H), 3.66-3.61 (m, 1H), 3.25-3.13 (m, 1H), 3.14-2.92 (m, 2H), 2.83-2.70 (m, 1H), 2.59-2.51 (m, 1H), 2.17-2.06 (m, 2H), 2.02 (s, 3H), 1.85 (dd, J=7.69, 13.27 Hz, 1H), 1.73-1.62 (m, 1H) (one proton was hidden under water peak); LCMS t₁ (Method 1)=3.776 min, m/z 337.2 [M+H⁺].

### N-Benzyl-1-((2,3-dihydrobenzo[b][1,4]dioxin-6-yl)methyl)-N-phenethylpiperidin-4-amine (NCGC00347050-01, XJB14-076)

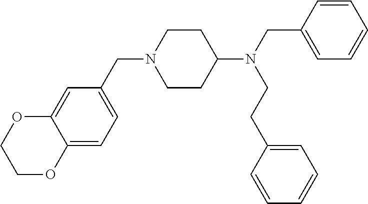 [SMILES: c1ccc(CCN(Cc2ccccc2)C2CCN(Cc3ccc4c(c3)OCCO4)CC2)cc1]

The title compound was prepared according to General Protocol A as a TFA salt. LCMS t₁ (Method 1)=3.747 min, m/z 443.3 [M+H⁺].

### 1-([1,1′-Biphenyl]-4-ylmethyl)-N-benzyl-N-phenethylpiperidin-4-amine (NCGC00347051-01, XJB14-077)

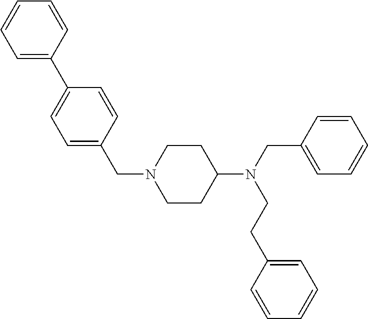 [SMILES: c1ccc(CCN(Cc2ccccc2)C2CCN(Cc3ccc(-c4ccccc4)cc3)CC2)cc1]

The title compound was prepared according to General Protocol A as a TFA salt. LCMS t₁ (Method 1)=4.354 min, m/z 461.3 [M+H⁺].

### N-Benzyl-1-(4-iodobenzyl)-N-phenethylpiperidin-4-amine (NCGC00347052-01, XJB14-074)

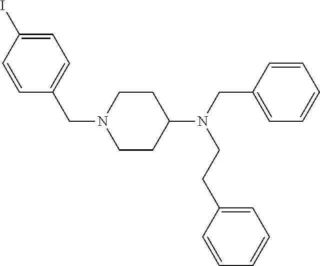 [SMILES: Ic1ccc(CN2CCC(N(CCc3ccccc3)Cc3ccccc3)CC2)cc1]

The title compound was prepared according to General Protocol A as a TFA salt. LCMS t₁ (Method 1)=4.094 min, m/z 511.2 [M+H⁺].

### N-Benzyl-1-(2-methoxybenzyl)-N-phenethylpiperidin-4-amine (NCGC00347053-01, XJB14-075)

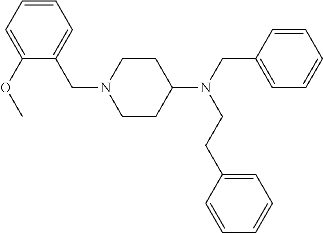 [SMILES: COc1ccccc1CN1CCC(N(CCc2ccccc2)Cc2ccccc2)CC1]

The title compound was prepared according to General Protocol A as a TFA salt. LCMS t₁ (Method 1)=3.783 min, m/z 415.3 [M+H⁺].

### 4-((4-(Benzyl(phenethyl)amino)piperidin-1-yl)methyl)benzonitrile (NCGC00347054-01, XJB14-058)

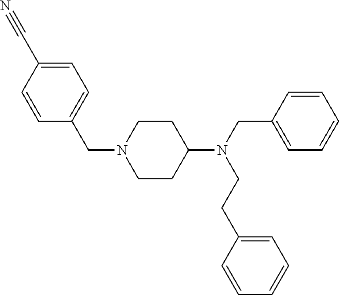 [SMILES: N#Cc1ccc(CN2CCC(N(CCc3ccccc3)Cc3ccccc3)CC2)cc1]

The title compound was prepared according to General Protocol A as a TFA salt. LCMS t₁ (Method 1)=3.823 min, m/z 410.2 [M+H⁺].

### N-Benzyl-1-(4-bromobenzyl)-N-phenethylpiperidin-4-amine (NCGC00347055-01, XJB14-056)

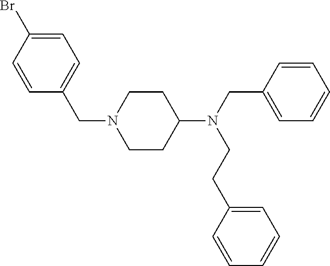 [SMILES: Brc1ccc(CN2CCC(N(CCc3ccccc3)Cc3ccccc3)CC2)cc1]

The title compound was prepared according to General Protocol A as a TFA salt. LCMS t₁ (Method 1)=3.960 min, m/z 463.1 [M+H⁺].

### N-Benzyl-1-(3-methoxybenzyl)-N-phenethylpiperidin-4-amine (NCGC00347056-01, XJB14-057)

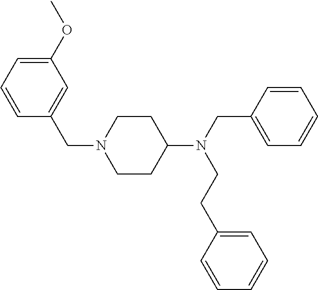 [SMILES: COc1cccc(CN2CCC(N(CCc3ccccc3)Cc3ccccc3)CC2)c1]

The title compound was prepared according to General Protocol A as a TFA salt. LCMS t₁ (Method 1)=3.769 min, m/z 415.2 [M+H⁺].

### N-Benzyl-1-(4-fluorobenzyl)-N-phenethylpiperidin-4-amine (NCGC00347057-01, XJB14-053)

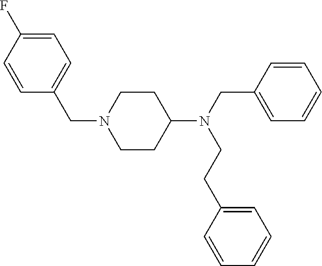 [SMILES: Fc1ccc(CN2CCC(N(CCc3ccccc3)Cc3ccccc3)CC2)cc1]

A solution of N-benzyl-N-phenethylpiperidin-4-amine (30.0 mg, 0.102 mmol) and 4-fluorobenzaldehyde (25.3 mg, 0.204 mmol) in ethanol (2.00 mL) was treated at room temperature with Ts-OH (2.9 mg, 0.015 mmol). The reaction mixture was stirred at room temperature for 10 min, NaCNBH₄ (64.0 mg, 1.02 mmol) was added. The resulting mixture was stirred at room temperature overnight. The mixture was quenched with 1.0 N NaOH aq. solution. The mixture was dried by blowing air, re-dissolved in DMSO, filtered and purified by HPLC to give the title compound as a TFA salt. LCMS t₁ (Method 1)=3.774 min, m/z 403.2 [M+H⁺].

### N-Benzyl-1-(4-methoxybenzyl)-N-phenethylpiperidin-4-amine (NCGC00347058-01, XJB14-054)

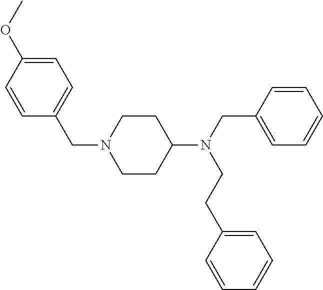 [SMILES: COc1ccc(CN2CCC(N(CCc3ccccc3)Cc3ccccc3)CC2)cc1]

A solution of N-benzyl-N-phenethylpiperidin-4-amine (30.0 mg, 0.102 mmol) and 4-methoxybenzaldehyde (27.7 mg, 0.204 mmol) in ethanol (2.00 mL) was treated at room temperature with Ts-OH (2.9 mg, 0.015 mmol). The reaction mixture was stirred at room temperature for 10 min, NaCNBH₄ (64.0 mg, 1.02 mmol) was added. The resulting mixture was stirred at room temperature overnight. The mixture was quenched with 1.0 N NaOH aq. solution. The mixture was dried by blowing air, re-dissolved in DMSO, filtered and purified by HPLC to give the title compound as a TFA salt. LCMS t₁ (Method 1)=3.874 min, m/z 415.2 [M+H⁺].

### N-Benzyl-N,1-diphenethylpiperidin-4-amine (NCGC00347059-01, XJB14-055)

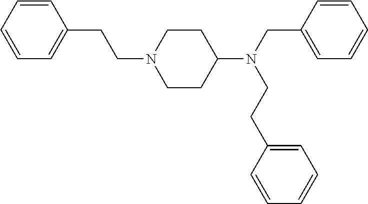 [SMILES: c1ccc(CCN2CCC(N(CCc3ccccc3)Cc3ccccc3)CC2)cc1]

A solution of N-benzyl-N-phenethylpiperidin-4-amine (30.0 mg, 0.102 mmol) and 2-phenylacetaldehyde (24.5 mg, 0.204 mmol) ine (2.00 mL) was treated at room temperature with Ts-OH (2.9 mg, 0.015 mmol). The reaction mixture was stirred at room temperature for 10 min, NaCNBH₄ (64.0 mg, 1.02 mmol) was added. The mixture was quenched with 1.0 N NaOH aq. solution. The mixture was dried by blowing air, re-dissolved in DMSO, filtered and purified by HPLC to give the title compound as a TFA salt. LCMS t₁ (Method 1)=3.865 min, m/z 399.2 [M+H⁺].

### Methyl 4-((4-(benzyl(phenethyl)amino)piperidin-1-yl)methyl)benzoate (NCGC00347206-01, XJB14-078)

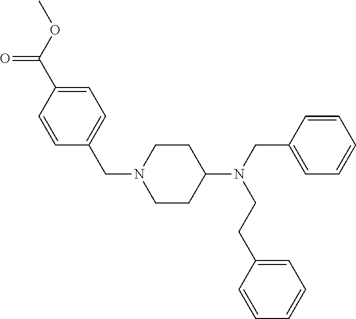 [SMILES: COC(=O)c1ccc(CN2CCC(N(CCc3ccccc3)Cc3ccccc3)CC2)cc1]

The title compound was prepared according to General Protocol A as a TFA salt. LCMS t₁ (Method 1)=3.825 min, m/z 443.3 [M+H⁺].

### N-Benzyl-N-phenethyl-1-propylpiperidin-4-amine (NCGC00347207-01, XJB015-002)

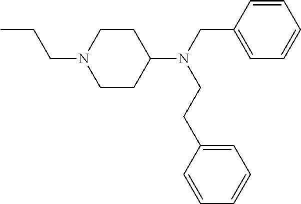 [SMILES: CCCN1CCC(N(CCc2ccccc2)Cc2ccccc2)CC1]

The title compound was prepared according to General Protocol B as a TFA salt. LCMS t₁ (Method 1)=3.436 min, m/z 337.2 [M+H⁺].

### N-Benzyl-1-butyl-N-phenethylpiperidin-4-amine (NCGC00347209-01, XJB015-008)

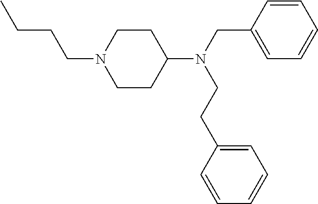 [SMILES: CCCCN1CCC(N(CCc2ccccc2)Cc2ccccc2)CC1]

The title compound was prepared according to General Protocol B as a TFA salt. LCMS t₁ (Method 1)=3.599 min, m/z 351.2 [M+H⁺].

### N-Benzyl-1-ethyl(2,2,2-d₃)-N-phenethylpiperidin-4-amine (XJB015-081)

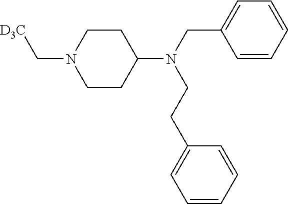 [SMILES: [2H]C([2H])([2H])CN1CCC(N(CCc2ccccc2)Cc2ccccc2)CC1]

The title compound was prepared according to General Protocol B as a TFA salt. LCMS t₁ (Method 1)=3.347 min, m/z 326.3 [M+H⁺].

### N-Benzyl-N-phenethyl-1-(2,2,2-trifluoroethyl)piperidin-4-amine (XJB015-083)

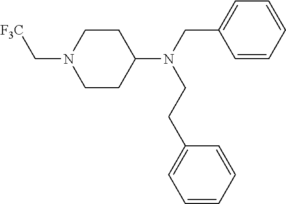 [SMILES: FC(F)(F)CN1CCC(N(CCc2ccccc2)Cc2ccccc2)CC1]

2,2,2-trifluoroethyl trifluoromethanesulfonate (23.7 mg, 0.102 mmol) was added to a stirred mixture of N-benzyl-N-phenethylpiperidin-4-amine (30.0 mg, 0.102 mmol), potassium carbonate (28.2 mg, 0.204 mmol) and Acetonitrile (1.00 mL). The reaction was stirred at room temperature for 5 hours. The mixture was dried by blowing air, re-dissolved in DMSO, filtered and purified by HPLC to give the title compound as a TFA salt. ¹H NMR (400 MHz, DMSO-d₆) δ 9.54 (s, 1H), 7.69-7.07 (m, 10H), 4.67-4.48 (m, 1H), 4.43-4.28 (m, 1H), 3.40-3.10 (m, 4H), 3.09-2.91 (m, 3H), 2.77 (tt, J=6.44, 12.86 Hz, 1H), 2.63-2.50 (m, 1H), 2.49-2.33 (m, 2H), 2.11-2.02 (m, 2H), 1.93-1.79 (m, 2H); LCMS t₁ (Method 1)=4.509 min, m/z 377.2 [M+H⁺].

### N-Benzyl-1-methyl(d₃)-N-phenethylpiperidin-4-amine (XJB015-078)

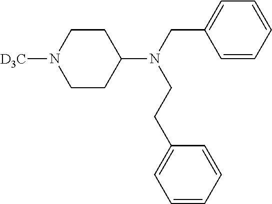 [SMILES: [2H]C([2H])([2H])N1CCC(N(CCc2ccccc2)Cc2ccccc2)CC1]

A solution of N-benzyl-N-phenethylpiperidin-4-amine (50.0 mg, 0.170 mmol) in THF (1.00 mL) and Water (0.500 mL) was treated at room temperature with NaOH (6.8 mg, 0.170 mmol) and MeI-d3 (10.6 μL, 0.170 mmol). The reaction mixture was stirred at 65° C. for 2 h. The mixture was cooled to room temperature, dried by blowing air, re-dissolved in DMSO, filtered and purified by HPLC under basic conditions to give the title compound.

### 1-Benzhydryl-4-methylpiperazine (NCGC00016421-01)

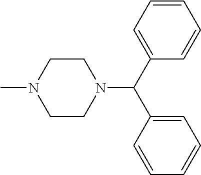 [SMILES: CN1CCN(C(c2ccccc2)c2ccccc2)CC1]

The title compound was purchased from Prestwick Chemical, Inc., CAS #303-25-3.

### 2-(2-(4-((4-Chlorophenyl)(phenyl)methyl)piperazin-1-yl)ethoxy) acetic acid (NCGC00016949-01)

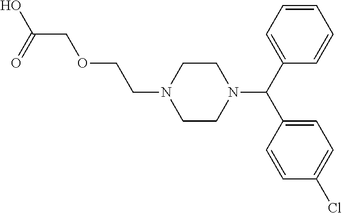 [SMILES: O=C(O)COCCN1CCN(C(c2ccccc2)c2ccc(Cl)cc2)CC1]

The title compound was purchased from Prestwick Chemical, Inc., CAS#83881-52-1.

### 2-(2-(4-((4-Chlorophenyl)(phenyl)methyl)piperazin-1-yl)ethoxy)ethanol (NCGC00018255-04)

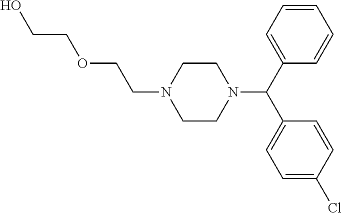 [SMILES: OCCOCCN1CCN(C(c2ccccc2)c2ccc(Cl)cc2)CC1]

The title compound was purchased from Timtec with catalog # ST059726.

### 2-(2-(4-((4-Chlorophenyl)(phenyl)methyl)piperazin-1-yl)ethoxy)acetamide (NCGC00181793-01)

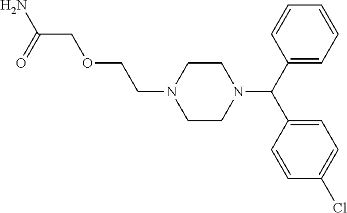 [SMILES: NC(=O)COCCN1CCN(C(c2ccccc2)c2ccc(Cl)cc2)CC1]

The title compound was purchased from Toronto Research with catalog # C291090.

### 1-((4-Chlorophenyl)(phenyl)methyl)-4-methyl-1,4-diazepane (NCGC00018271-03)

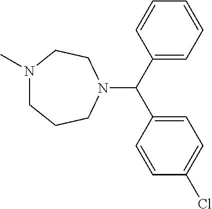 [SMILES: CN1CCCN(C(c2ccccc2)c2ccc(Cl)cc2)CC1]

The title compound was purchased from BIOMOL with catalog # AC-927.

### 1-((4-Chlorophenyl)(phenyl)methyl)-4-methylpiperazine (NCGC00179384-04)

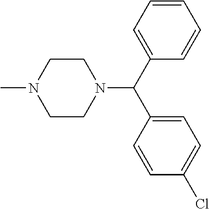 [SMILES: CN1CCN(C(c2ccccc2)c2ccc(Cl)cc2)CC1]

The title compound was purchased from MP Biomedicals.

### (R)-1-((4-Chlorophenyl)(phenyl)methyl)-4-methylpiperazine (NCGC00343774-03, XJB13-077)

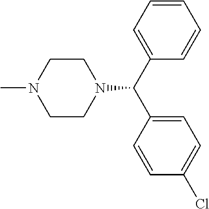 [SMILES: CN1CCN([C@H](c2ccccc2)c2ccc(Cl)cc2)CC1]

The title compound was purified to >99% purity using supercritical fluid chromatography (SFC) preparative systems at Lotus Separations, LLC (Princeton, N.J., USA). For preparative separation, an AD-H (2×15 cm) column was used with an eluent of 25% isopropanol (0.1% DEA)/CO₂, 100 bar. Flow rate was 70 mL/min and detection wavelength was 220 nm. For analytical separation, an AD-H (25×0.46 cm) column was used with an eluent of 40% isopropanol/CO₂, 100 bar. Flow rate was 3.0 mL/min and detection wavelengths were 220 and 280 nm. Retention time for R-configuration enantiomer was 2.15 min with negative optical rotation. Retention time for S-configuration enantiomer was 2.63 min with positive optical rotation.

The title compound also can be prepared by chemical synthesis. A solution of (R)-1-((4-chlorophenyl)(phenyl)methyl)piperazine (50.0 mg, 0.174 mmol) in THF (1.00 mL) and water (0.50 mL) was treated at room temperature with NaOH (6.97 mg, 0.174 mmol) and MeI (10.9 μL, 0.174 mmol). The reaction mixture was stirred at 65° C. for 2 h. The reaction mixture was cooled to room temperature. The organic layer was separated, dried, concentrated and purified by Biotage on SiO₂ with 0-20% of MeOH in CH₂Cl₂ to give the title compound as a white solid. LCMS t₂ (Method 2)=3.070 min; m/z 301.1 [M+H⁺].

### (S)-1-((4-Chlorophenyl)(phenyl)methyl)-4-methylpiperazine (NCGC00343775-03, XJB13-076)

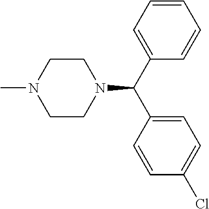 [SMILES: CN1CCN([C@@H](c2ccccc2)c2ccc(Cl)cc2)CC1]

The title enantiomerically pure compound was purified to >99% purity using supercritical fluid chromatography (SFC) preparative systems at Lotus Separations, LLC (Princeton, N.J., USA). This enantiomer has a retention time of 2.63 min with positive optical rotation.

The title compound also can be prepared by chemical synthesis. A solution of (S)-1-((4-chlorophenyl)(phenyl)methyl)piperazine (50.0 mg, 0.174 mmol) in THF (1.00 mL) and water (0.50 mL) was treated at room temperature with NaOH (6.97 mg, 0.174 mmol) and MeI (10.9 μL, 0.174 mmol). The reaction mixture was stirred at 65° C. for 2 h. The reaction mixture was cooled to room temperature. The organic layer was separated, dried, concentrated and purified by Biotage on SiO₂ with 0-20% of MeOH in CH₂Cl₂ to give the title compound as a white solid. LCMS t₂ (Method 2)=3.093 min; m/z 301.1 [M+H⁺].

### (R)-1-((4-Chlorophenyl)(phenyl)methyl)piperazine (NCGC00345879-01)

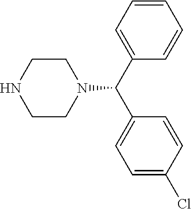 [SMILES: Clc1ccc([C@@H](c2ccccc2)N2CCNCC2)cc1]

The title compound was purchased from Albany Molecule with catalog # A00156.

### (S)-1-((4-Chlorophenyl)(phenyl)methyl)piperazine (NCGC00345880-01)

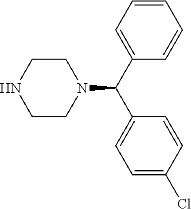 [SMILES: Clc1ccc([C@H](c2ccccc2)N2CCNCC2)cc1]

The title compound was purchased from Albany Molecule with catalog # A00156-1.

### (S)-1-((4-Chlorophenyl)(phenyl)methyl)-4-(2,4-dimethoxybenzyl)piperazine (NCGC00346845-01, XJB14-024)

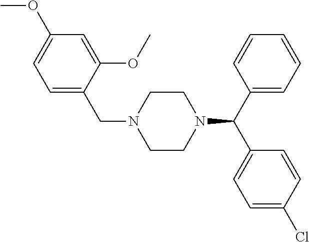 [SMILES: COc1ccc(CN2CCN([C@@H](c3ccccc3)c3ccc(Cl)cc3)CC2)c(OC)c1]

A solution of (S)-1-((4-chlorophenyl)(phenyl)methyl)piperazine (45.0 mg, 0.157 mmol) and 2,4-dimethoxybenzaldehyde (52.1 mg, 0.314 mmol) in ethanol (2.00 mL) was treated at room temperature with titanium (IV) isopropoxide (0.092 mL, 0.314 mmol). The reaction mixture was stirred at room temperature for 10 min and treated with NaCNBH₄ (49.3 mg, 0.785 mmol). The resulting mixture was stirred at room temperature for 1 h and quenched at room temperature with 1 N NaOH. The mixture was dried by blowing air, re-dissolved in DMSO, filtered and purified by preparative HPLC to give the final product as a TFA salt. ¹H NMR (400 MHz, DMSO-d₆) δ 9.22 (s, 1H), 7.48-7.25 (m, 9H), 7.28-7.14 (m, 1H), 6.68-6.56 (m, 2H), 4.51 (s, 1H), 4.20 (d, J=4.69 Hz, 2H), 3.82 (s, 3H), 3.79 (s, 3H), 3.29 (d, J=12.47 Hz, 1H), 3.07 (q, J=10.83 Hz, 2H), 2.85-2.77 (m, 2H), 2.24 (s, 2H); LCMS t₁ (Method 1)=5.552 min, m/z 437.1 [M+H⁺].

### (S)-1-((4-Chlorophenyl)(phenyl)methyl)-4-ethylpiperazine (NCGC00346848-01, XJB14-028, XJB15-076)

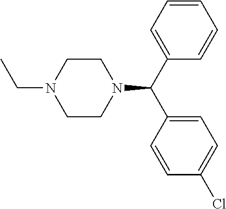 [SMILES: CCN1CCN([C@@H](c2ccccc2)c2ccc(Cl)cc2)CC1]

The title compound was prepared according to General Protocol B as a TFA salt. ¹H NMR (400 MHz, DMSO-d₆) δ 9.22 (s, 1H), 7.50-7.29 (m, 8H), 7.29-7.19 (m, 1H), 4.54 (s, 1H), 3.42 (d, J=12.23 Hz, 2H), 3.18-3.09 (m, 2H), 3.04 (q, J=11.21 Hz, 2H), 2.84 (d, J=13.01 Hz, 2H), 2.21 (q, J=11.50 Hz, 2H), 1.18 (t, J=7.27 Hz, 3H); LCMS t₁ (Method 1)=4.566 min; t₂ (Method 2)=3.035 min, m/z 315.1 [M+H⁺].

### N-(4-(tert-Butoxy)phenyl)-1-methyl-N-phenylpiperidin-4-amine (NCGC00346851-01, XJB14-042)

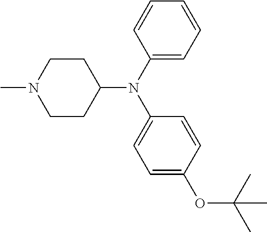 [SMILES: CN1CCC(N(c2ccccc2)c2ccc(OC(C)(C)C)cc2)CC1]

A mixture of N-(4-chlorophenyl)-1-methylpiperidin-4-amine (30.0 mg, 0.133 mmol), iodobenzene (0.030 mL, 0.267 mmol), Pd(OAc)₂ (3.00 mg, 0.013 mmol), BINAP (9.14 mg, 0.015 mmol), and potassium tert-butoxide (18.7 mg, 0.167 mmol) (0.167 mmol, 1.0 M solution in THF, 0.167 mL) in toluene (0.200 mL) was stirred at 110° C. for 4 h. The reaction was cooled to room temperature and treated with Si-Thiol. The mixture was dried by blowing air, re-dissolved in DMSO, filtered and purified by preparative HPLC to give the final product as a TFA salt.). LCMS t₁ (Method 1)=4.656 min, m/z 339.1 [M+H⁺].

### (S)-1-((4-Chlorophenyl)(phenyl)methyl)-4-cyclopentylpiperazine (NCGC00347036-01, XJB14-069)

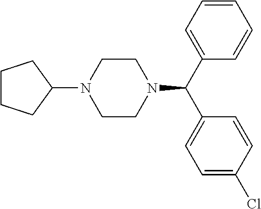 [SMILES: Clc1ccc([C@H](c2ccccc2)N2CCN(C3CCCC3)CC2)cc1]

The title compound was prepared according to General Protocol A as a TFA salt. ¹H NMR (400 MHz, DMSO-d₆) δ 9.25 (s, 1H), 7.50-7.29 (m, 8H), 7.31-7.20 (m, 1H), 4.55 (s, 1H), 3.57-3.40 (m, 3H), 3.16-3.02 (m, 2H), 2.85 (d, J=12.86 Hz, 2H), 2.28-2.14 (m, 2H), 2.04-1.90 (m, 2H), 1.73-1.47 (m, 6H); LCMS t₁ (Method 1)=4.871 min; t₂ (Method 2)=3.149 min, m/z 355.1 [M+H⁺].

### (S)-1-((4-Chlorophenyl)(phenyl)methyl)-4-cyclohexylpiperazine (NCGC00347039-01, XJB14-084)

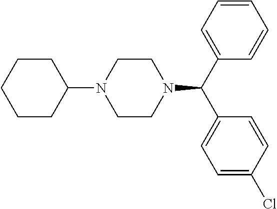 [SMILES: Clc1ccc([C@H](c2ccccc2)N2CCN(C3CCCCC3)CC2)cc1]

The title compound was prepared according to General Protocol A as a TFA salt. ¹H NMR (400 MHz, DMSO-d₆) δ 9.08 (s, 1H), 7.50-7.29 (m, 8H), 7.29-7.20 (m, 1H), 4.54 (s, 1H), 3.17-3.04 (m, 3H), 2.86 (d, J=12.75 Hz, 2H), 2.25 (q, J=11.46 Hz, 2H), 2.03 (d, J=11.14 Hz, 2H), 1.81 (d, J=12.56 Hz, 2H), 1.61 (d, J=12.82 Hz, 1H), 1.40-1.16 (m, 4H), 1.15-1.01 (m, 1H) (two protons were hidden under the water peak); ¹⁹F NMR (376 MHz, DMSO-d₆) δ −73.56; LCMS t₁, (Method 1)=5.048 min; m/z 369.2 [M+H⁺].

### (R)-1-((4-Chlorophenyl)(phenyl)methyl)-4-ethylpiperazine (NCGC00347040-01, XJB14-085)

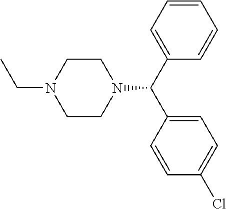 [SMILES: CCN1CCN([C@H](c2ccccc2)c2ccc(Cl)cc2)CC1]

The title compound was prepared according to General Protocol B as a TFA salt. ¹H NMR (400 MHz, DMSO-d₆) δ 9.18 (s, 1H), 7.48-7.27 (m, 8H), 7.27-7.18 (m, 4.52 (s, 1H), 3.40 (d, J=11.93 Hz, 2H), 3.16-2.95 (m, 4H), 2.83 (d, J=13.06 Hz, 2H), 2.19 (q, J=11.58 Hz, 2H), 1.17 (t, J=7.25 Hz, 3H); ¹⁹F NMR (376 MHz, DMSO-d₆) δ −73.48; LCMS t₁ (Method 1)=4.505 min, m/z 315.1 [M+H⁺].

### (S)-1-((4-Chlorophenyl)(phenyl)methyl)-4-isobutylpiperazine (NCGC00347042-01, XJB14-087)

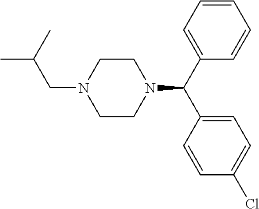 [SMILES: CC(C)CN1CCN([C@@H](c2ccccc2)c2ccc(Cl)cc2)CC1]

The title compound was prepared according to General Protocol B as a TFA salt. ¹H NMR (400 MHz, DMSO-d₆) δ 8.92 (s, 1H), 7.47-7.28 (m, 8H), 7.27-7.18 (m, 1H), 4.54 (s, 1H), 3.45-3.36 (m, 2H), 3.05 (q, J=11.14 Hz, 2H), 2.93 (dd, J=5.50, 7.14 Hz, 2H), 2.82-2.74 (m, 2H), 2.37-2.25 (m, 2H), 2.00 (hept, J=6.78 Hz, 1H), 0.91 (d, J=6.60 Hz, 6H); ¹⁹F NMR (376 MHz, DMSO-d₆) δ −73.54; LCMS t₁ (Method 1)=4.858 min, m/z 343.2 [M+H⁺].

### (S)-1-((4-Chlorophenyl)(phenyl)methyl)-4-isopropylpiperazine (NCGC00347044-01, XJB14-067)

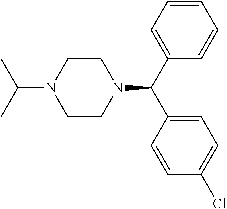 [SMILES: CC(C)N1CCN([C@@H](c2ccccc2)c2ccc(Cl)cc2)CC1]

A solution of (S)-1-((4-chlorophenyl)(phenyl)methyl)piperazine (30.0 mg, 0.105 mmol) and acetone (60.8 mg, 1.046 mmol) in ethanol (2.00 mL) was treated at room temperature with Ts-OH (2.98 mg, 0.016 mmol). The reaction mixture was stirred at room temperature for 10 min, NaCNBH₄ (65.7 mg, 1.05 mmol) was added. The reaction mixture was stirred at room temperature overnight and quenched with 1 N NaOH solution. The mixture was dried by blowing air, re-dissolved in DMSO, filtered and purified by HPLC to give the title compound as a TFA salt. ¹H NMR (400 MHz, DMSO-d₆) δ 9.05 (s, 1H), 7.50-7.29 (m, 8H), 7.29-7.20 (m, 1H), 4.55 (s, 1H), 3.51-3.40 (m, 3H), 3.08 (q, J=11.33 Hz, 2H), 2.87 (d, J=12.96 Hz, 2H), 2.23 (q, J=11.23 Hz, 2H), 1.24 (d, J=6.56 Hz, 6H); ¹⁹F NMR (376 MHz, DMSO-d₆) δ −73.56; LCMS t₁, (Method 1)=4.688 min, m/z 329.1 [M+H⁺].

### (S)-1-(4-((4-Chlorophenyl)(phenyl)methyl)piperazin-1-yl)ethanone (NCGC00347049-01, XJB14-071)

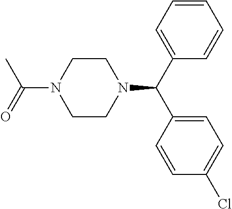 [SMILES: CC(=O)N1CCN([C@@H](c2ccccc2)c2ccc(Cl)cc2)CC1]

A solution of (S)-1-((4-chlorophenyl)(phenyl)methyl)piperazine (30.0 mg, 0.105 mmol) in dichloromethane (2.00 mL) was treated at room temperature with acetyl chloride (16.4 mg, 0.209 mmol) and triethylamine (0.044 mL, 0.314 mmol). The reaction mixture was stirred at room temperature for 1 h. The mixture was dried by blowing air, re-dissolved in DMSO, filtered and purified by HPLC to give the title compound as a TFA salt. LCMS t₁ (Method 1)=4.123 min, m/z 329.1 [M+H⁺].

### (R)-1-((4-Chlorophenyl)(phenyl)methyl)-4-isobutylpiperazine (NCGC00347205-01, XJB14-092)

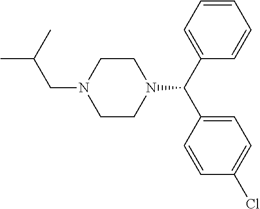 [SMILES: CC(C)CN1CCN([C@H](c2ccccc2)c2ccc(Cl)cc2)CC1]

The title compound was prepared according to General Protocol B as a TFA salt. ¹H NMR (400 MHz, DMSO-d₆) δ 8.92 (s, 1H), 7.49-7.30 (m, 8H), 7.29-7.20 (m, 1H), 4.56 (s, 1H), 3.42 (d, J=11.68 Hz, 2H), 3.07 (q, J=11.05 Hz, 2H), 2.95 (dd, J=5.41, 7.28 Hz, 2H), 2.84-2.76 (m, 2H), 2.33 (q, J=11.37 Hz, 2H), 2.02 (hept, J=6.76 Hz, 1H), 0.93 (d, J=6.58 Hz, 6H); ¹⁹F NMR (376 MHz, DMSO-d₆) δ −73.62; LCMS t₁ (Method 1)=4.881 min, m/z 343.2 [M+H⁺].

### (S)-1-Butyl-4-((4-chlorophenyl)(phenyl)methyl)piperazine (NCGC00347208-01, XJB015-007)

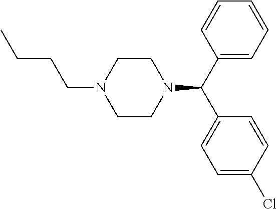 [SMILES: CCCCN1CCN([C@@H](c2ccccc2)c2ccc(Cl)cc2)CC1]

The title compound was prepared according to General Protocol B as a TFA salt. ¹H NMR (400 MHz, DMSO-d₆) δ 9.23 (s, 1H), 7.49-7.29 (m, 8H), 7.29-7.19 (m, 1H), 4.53 (s, 1H), 3.43 (d, J=10.81 Hz, 2H), 3.06 (dt, J=5.09, 11.92 Hz, 4H), 2.83 (d, J=13.08 Hz, al), 2.23 (td, J=6.99, 11.94 Hz, 2H), 1.57 (tt, J=6.23, 8.00 Hz, 2H), 1.30 (h, J=7.36 Hz, 2H), 0.90 (t, J=7.34 Hz, 3H); ¹⁹F NMR (376 MHz, DMSO-d₆) δ −73.63; LCMS t₁, (Method 1)=5.015 min, m/z 343.1 [M+H⁺].

### (R)-1-Butyl-4-((4-chlorophenyl)(phenyl)methyl)piperazine (NCGC00347210-01, XJB015-009)

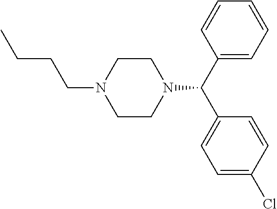 [SMILES: CCCCN1CCN([C@H](c2ccccc2)c2ccc(Cl)cc2)CC1]

The title compound was prepared according to General Protocol B as a TFA salt. ¹H NMR (400 MHz, DMSO-d₆) δ 9.23 (s, 1H), 7.49-7.29 (m, 8H), 7.29-7.19 (m, 1H), 4.53 (s, 1H), 3.43 (d, J=12.96 Hz, 2H), 3.07 (dq, J=4.91, 11.93 Hz, 4H), 2.87-2.79 (m, 2H), 2.29-2.16 (m, 2H), 1.64-1.51 (m, 2H), 1.30 (h, J=7.40 Hz, 2H), 0.90 (t, J=7.34 Hz, 3H); ¹⁹F NMR (376 MHz, DMSO-d₆) δ −73.63; LCMS t₁, (Method 1)=5.038 min, m/z 343.1 [M+H⁺].

### (R)-1-((4-Chlorophenyl)(phenyl)methyl)-4-propylpiperazine (NCGC00347960-01, XJB015-003)

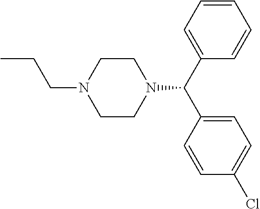 [SMILES: CCCN1CCN([C@H](c2ccccc2)c2ccc(Cl)cc2)CC1]

The title compound was prepared according to General Protocol B as a TFA salt. ¹H NMR (400 MHz, DMSO-d₆) δ 9.20 (d, J=9.36 Hz, 1H), 7.49-7.29 (m, 8H), 7.29-7.20 (m, 1H), 4.54 (s, 1H), 3.42 (d, J=12.10 Hz, 2H), 3.12-2.97 (m, 4H), 2.83 (d, J=13.02 Hz, 2H), 2.29-2.16 (m, 2H), 1.69-1.54 (m, 2H), 0.89 (t, J=7.38 Hz, 3H); ¹⁹F NMR (376 MHz, DMSO-d₆) δ −73.55; LCMS t₁ (Method 1)=4.746 min, m/z 329.1 [M+H⁺].

### (S)-1-((4-Chlorophenyl)(phenyl)methyl)-4-propylpiperazine (NCGC00347959-01, XJB015-004)

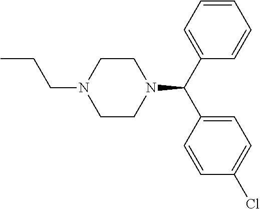 [SMILES: CCCN1CCN([C@@H](c2ccccc2)c2ccc(Cl)cc2)CC1]

The title compound was prepared according to General Protocol A as a TFA salt. ¹H NMR (400 MHz, DMSO-d₆) δ 9.20 (s, 1H), 7.49-7.29 (m, 8H), 7.29-7.19 (m, 1H), 4.54 (s, 1H), 3.42 (d, J=12.04 Hz, 2H), 3.12-2.97 (m, 4H), 2.83 (d, J=12.71 Hz, 2H), 2.23 (q, J=11.27 Hz, 2H), 1.69-1.54 (m, 2H), 0.89 (t, J=7.37 Hz, 3H); ¹⁹F NMR (376 MHz, DMSO-d₆) δ −73.46; LCMS t₁, (Method 1)=4.817 min, m/z 329.1 [M+H⁺].

### (R)-1-((4-Chlorophenyl)(phenyl)methyl)-4-ethyl(2,2,2-d₃)piperazine (XJB015-080, NCGC00350944-02)

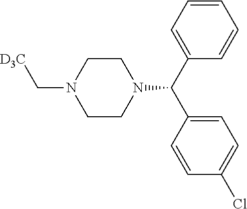 [SMILES: [2H]C([2H])([2H])CN1CCN([C@H](c2ccccc2)c2ccc(Cl)cc2)CC1]

The title compound was prepared according to General Protocol B. ¹H NMR (400 MHz, DMSO-d₆) δ 7.48-7.14 (m, 9H), 4.29 (s, 1H), 2.38 (s, 4H), 2.34-2.20 (m, 6H); LCMS t₁ (Method 1)=4.630 min, m/z 317.2 [M+H⁺].

### (S)-1-((4-Chlorophenyl)(phenyl)methyl)-4-ethyl(2,2,2-d₃)piperazine (XJB015-060, NCGC00351278-02)

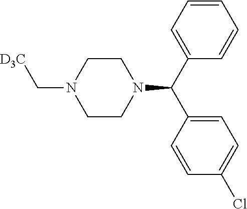 [SMILES: [2H]C([2H])([2H])CN1CCN([C@@H](c2ccccc2)c2ccc(Cl)cc2)CC1]

The title compound was prepared according to General Protocol B. LCMS t₁, (Method 1)=4.671 min, m/z 317.2 [M+H⁺].

### (R)-1-((4-Chlorophenyl)(phenyl)methyl)-4-(2,2,2-trifluoroethyl)piperazine (XJB015-062, XJB015-082, NCGC00350946-02)

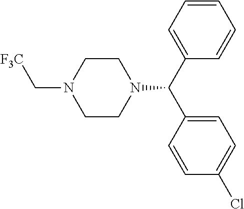 [SMILES: FC(F)(F)CN1CCN([C@H](c2ccccc2)c2ccc(Cl)cc2)CC1]

2,2,2-Trifluoroethyl trifluoromethanesulfonate (24.3 mg, 0.105 mmol) was added to a stirred mixture of (R)-1-((4-chlorophenyl)(phenyl)methyl)piperazine (30.0 mg, 0.105 mmol), potassium carbonate (28.9 mg, 0.209 mmol) and acetonitrile (1.00 mL). The reaction mixture was stirred at room temperature for 5 h. The mixture was dried by blowing air, re-dissolved in DMSO, filtered and purified by HPLC to give the title compound as a TFA salt. LCMS t₁ (Method 1)=4.846 min, m/z 369.1 [M+H⁺].

### (S)-1-((4-Chlorophenyl)(phenyl)methyl)-4-(2,2,2-trifluoroethyl)piperazine (XJB015-064, XJB015-084, NCGC00351281-01)

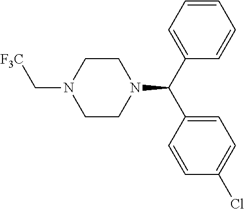 [SMILES: FC(F)(F)CN1CCN([C@@H](c2ccccc2)c2ccc(Cl)cc2)CC1]

2,2,2-Trifluoroethyl trifluoromethanesulfonate (24.3 mg, 0.105 mmol) was added to a stirred mixture of (S)-1-((4-chlorophenyl)(phenyl)methyl)piperazine (30.0 mg, 0.105 mmol), potassium carbonate (28.9 mg, 0.209 mmol) and acetonitrile (1.00 mL). The reaction mixture was stirred at room temperature for 5 h. The mixture was dried by blowing air, re-dissolved in DMSO, filtered and purified by HPLC to give the title compound as a TFA salt. LCMS t₁ (Method 1)=3.160 min, m/z 369.1 [M+H⁺].

### (R)-1-((4-Chlorophenyl)(phenyl)methyl)-4-methyl(d₃)piperazine (XJB015-075, NCGC00350947-01)

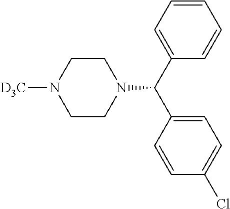 [SMILES: [2H]C([2H])([2H])N1CCN([C@H](c2ccccc2)c2ccc(Cl)cc2)CC1]

A solution of (R)-1-((4-chlorophenyl)(phenyl)methyl)piperazine (50.0 mg, 0.174 mmol) in THF (1.00 mL) and water (0.500 mL) was treated at room temperature with NaOH (7.0 mg, 0.174 mmol) and MeI-d₃ (10.9 μL, 0.174 mmol). The reaction mixture was stirred at 65° C. for 2 h. The mixture was dried by blowing air, re-dissolved in DMSO, filtered and purified by HPLC to give the title compound as a TFA salt. LCMS t₁ (Method 1)=4.484 min, m/z 304.1 [M+H⁺].

### (S)-1-((4-Chlorophenyl)(phenyl)methyl)-4-methyl(d₃)piperazine (XJB015-089, NCGC00351279-01)

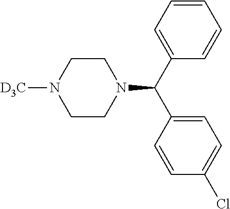 [SMILES: [2H]C([2H])([2H])N1CCN([C@@H](c2ccccc2)c2ccc(Cl)cc2)CC1]

A solution of (S)-1-((4-chlorophenyl)(phenyl)methyl)piperazine (30.0 mg, 0.105 mmol) in THF (1.00 mL) and water (0.500 mL) was treated at room temperature with NaOH (4.2 mg, 0.105 mmol) and MeI-d₃ (6.5 μL, 0.105 mmol). The reaction mixture was stirred at 65° C. for 2 h. The mixture was dried by blowing air, re-dissolved in DMSO, filtered and purified by HPLC to give the title compound as a free base. LCMS t₁ (Method 1)=4.501 min, m/z 304.1 [M+H⁺].

### N-Benzyl-1-(2,4-dimethoxybenzyl)-N-phenethylpiperidin-4-amine (NCGC00345021-03, hit, XJB14-029)

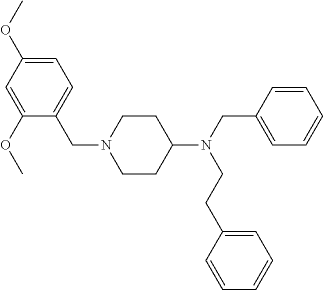 [SMILES: COc1ccc(CN2CCC(N(CCc3ccccc3)Cc3ccccc3)CC2)c(OC)c1]

The title compound was prepared according to General Protocol B as a TFA salt. LCMS t₁ (Method 1)=3.990 min, m/z 445.2 [M+H⁺].

### N-Benzyl-N-phenethylpiperidin-4-amine (NCGC00346843-01, XJB14-021)

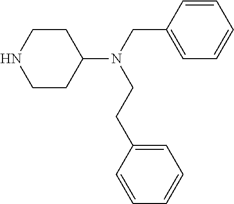 [SMILES: c1ccc(CCN(Cc2ccccc2)C2CCNCC2)cc1]

The title compound as HCl salt was purchased from ChemBridge, catalog #6766468. The sample was converted to TFA salt using reverse phase HPLC. LCMS t₁ (Method 1)=3.276 min, m/z 295.1 [M+H⁺].

### (4-(Benzyl(phenethyl)amino)piperidin-1-yl)(phenyl)methanone (NCGC00346844-01, XJB14-022)

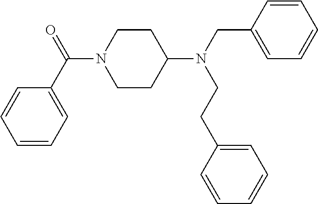 [SMILES: O=C(c1ccccc1)N1CCC(N(CCc2ccccc2)Cc2ccccc2)CC1]

A solution of N-benzyl-N-phenethylpiperidin-4-amine (30.0 mg, 0.102 mmol) in dichloromethane (2.00 mL) was treated at room temperature with triethylamine (0.071 mL, 0.509 mmol) followed by benzoyl chloride (28.6 mg, 0.204 mmol). The reaction mixture was stirred at room temperature overnight. The mixture was dried by blowing air, re-dissolved in DMSO, filtered and purified by HPLC to give the title compound as a TFA salt. ¹H NMR (400 MHz, DMSO-d₆) δ 9.49 (s, 1H), 7.65-7.58 (m, 2H), 7.57-7.13 (m, 13H), 4.59 (dd, J=3.79, 13.32 Hz, 1H), 4.37 (dd, J=6.68, 13.36 Hz, 1H), 3.79-3.61 (m, 3H), 3.20 (td, J=6.00, 12.11, 12.79 Hz, 2H), 2.99 (td, J=5.10, 12.65 Hz, 2H), 2.82-2.73 (m, 2H), 1.93-1.79 (m, 4H); LCMS t₁ (Method 1)=4.375 min, m/z 399.2 [M+H⁺].

### N-Benzyl-1-methyl-N-phenethylpiperidin-4-amine (NCGC00346846-01, XJB14-026)

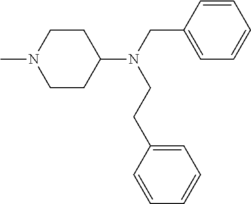 [SMILES: CN1CCC(N(CCc2ccccc2)Cc2ccccc2)CC1]

The title compound was prepared according to General Protocol B as a TFA salt. LCMS t₁ (Method 1)=3.422 min, m/z 309.2 [M+H⁺].

### N-Benzyl-1-ethyl-N-phenethylpiperidin-4-amine (NCGC00346847-01, XJB14-027, XJB015-074)

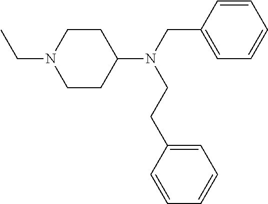 [SMILES: CCN1CCC(N(CCc2ccccc2)Cc2ccccc2)CC1]

The title compound was prepared according to General Protocol B as a TFA salt. LCMS t₁ (Method 1)=3.345 min, m/z 323.2 [M+H⁺].

### N-Benzyl-N-phenethyl-1-(phenylsulfonyl)piperidin-4-amine (NCGC00346849-01, XJB14-035)

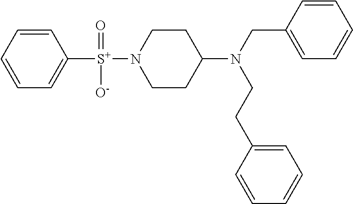 [SMILES: O=[S+]([O-])(c1ccccc1)N1CCC(N(CCc2ccccc2)Cc2ccccc2)CC1]

A solution of N-benzyl-N-phenethylpiperidin-4-amine (30.0 mg, 0.102 mmol) in dichloromethane (2.00 mL) was treated at room temperature with triethylamine (0.071 mL, 0.509 mmol) followed by benzenesulfonyl chloride (36.0 mg, 0.204 mmol). The reaction mixture was stirred at room temperature overnight. The mixture was dried by blowing air, re-dissolved in DMSO, filtered and purified by HPLC to give the title compound as a TFA salt. TFA salt. LCMS t₁ (Method 1)=4.648 min, m/z 435.2 [M+H⁺].

### N,1-dibenzyl-N-phenethylpiperidin-4-amine (NCGC00346850-01, XJB14-036)

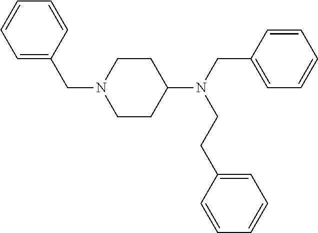 [SMILES: c1ccc(CCN(Cc2ccccc2)C2CCN(Cc3ccccc3)CC2)cc1]

The title compound was prepared according to General Protocol A as a TFA salt. LCMS t₁ (Method 1)=3.701 min, m/z 385.2 [M+H⁺].

### N-(4-(tert-Butoxy)phenyl)-1-methyl-N-phenylpiperidin-4-amine (NCGC00346851-01, XJB14-042)

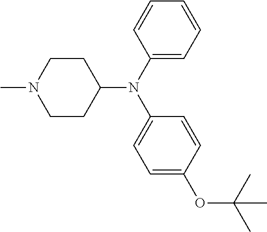 [SMILES: CN1CCC(N(c2ccccc2)c2ccc(OC(C)(C)C)cc2)CC1]

A mixture of N-(4-chlorophenyl)-1-methylpiperidin-4-amine (30.0 mg, 0.133 mmol), iodobenzene (0.030 mL, 0.267 mmol), Pd(OAc)₂ (3.00 mg, 0.013 mmol), BINAP (9.14 mg, 0.015 mmol) in toluene (0.200 mL) was treated at room temperature with potassium tert-butoxide (0.167 mL, 1.0 M solution in THF, 0.167 mmol). The reaction mixture was stirred at 110° C. for 4 h. The mixture was cooled to room temperature, dried by blowing air, re-dissolved in DMSO, filtered and purified by HPLC to give the title compound as a TFA salt. LCMS t₁ (Method 1)=4.656 min, m/z 339.1 [M+H⁺].

### N-Benzyl-1-cyclopentyl-N-phenethylpiperidin-4-amine (NCGC00347035-01, XJB14-068)

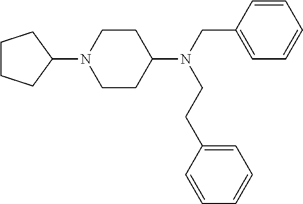 [SMILES: c1ccc(CCN(Cc2ccccc2)C2CCN(C3CCCC3)CC2)cc1]

The title compound was prepared according to General Protocol A as a TFA salt. LCMS t₁ (Method 1)=3.476 min, m/z 363.2 [M+H⁺].

### N-Benzyl-1-(4-methylbenzyl)-N-phenethylpiperidin-4-amine (NCGC00347037-01, XJB14-072)

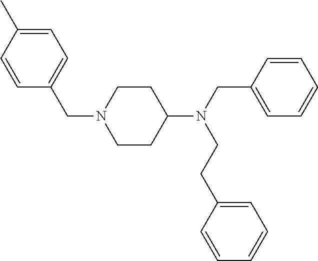 [SMILES: Cc1ccc(CN2CCC(N(CCc3ccccc3)Cc3ccccc3)CC2)cc1]

The title compound was prepared according to General Protocol A as a TFA salt. LCMS t₁ (Method 1)=3.864 min, m/z 399.3 [M+H⁺].

### N-Benzyl-1-(4-chlorobenzyl)-N-phenethylpiperidin-4-amine (NCGC00347038-01, XJB14-073)

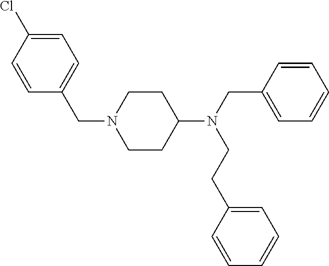 [SMILES: Clc1ccc(CN2CCC(N(CCc3ccccc3)Cc3ccccc3)CC2)cc1]

The title compound was prepared according to General Protocol A as a TFA salt. LCMS t₁ (Method 1)=3.933 min, m/z 419.2 [M+H⁺].

### N-Benzyl-1-isobutyl-N-phenethylpiperidin-4-amine (NCGC00347041-01, XJB14-086)

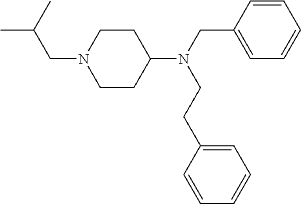 [SMILES: CC(C)CN1CCC(N(CCc2ccccc2)Cc2ccccc2)CC1]

The title compound was prepared according to General Protocol B as a TFA salt. LCMS t₁ (Method 1)=3.496 min, m/z 351.3 [M+H⁺].

### N-Benzyl-1-isopropyl-N-phenethylpiperidin-4-amine (NCGC00347043-01, XJB14-066)

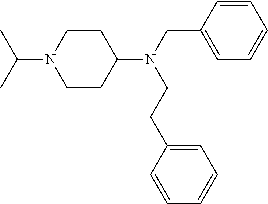 [SMILES: CC(C)N1CCC(N(CCc2ccccc2)Cc2ccccc2)CC1]

A solution of N-benzyl-N-phenethylpiperidin-4-amine (30.0 mg, 0.102 mmol) and propan-2-one (59.2 mg, 1.019 mmol) in ethanol (2.00 mL) was treated at room temperature with Ts-OH (2.91 mg, 0.015 mmol). The reaction mixture was stirred at room temperature for 10 min, then NaCNBH₄ (64.0 mg, 1.019 mmol) was added. The reaction mixture was stirred at room temperature over night. The mixture was cooled to room temperature, dried by blowing air, re-dissolved in DMSO, filtered and purified by HPLC to give the title compound as a TFA salt. LCMS t₁ (Method 1)=3.340 min, m/z 337.2 [M+H⁺].

### N-Benzyl-N-phenethyl-1-(4-(trifluoromethyl)benzyl)piperidin-4-amine (NCGC00347045-01, XJB14-063)

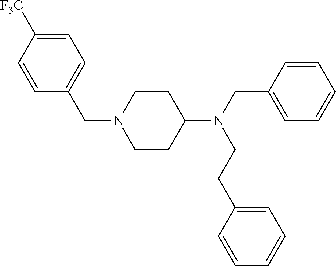 [SMILES: FC(F)(F)c1ccc(CN2CCC(N(CCc3ccccc3)Cc3ccccc3)CC2)cc1]

The title compound was prepared according to General Protocol A as a TFA salt.

### N-Benzyl-1-cyclohexyl-N-phenethylpiperidin-4-amine (NCGC00347046-01, XJB14-049)

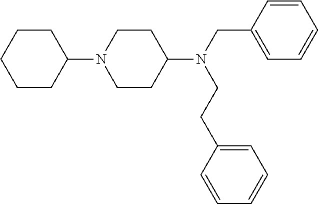 [SMILES: c1ccc(CCN(Cc2ccccc2)C2CCN(C3CCCCC3)CC2)cc1]

The title compound was prepared according to General Protocol A as a TFA salt. LCMS t₁ (Method 1)=3.647 min, m/z 377.2 [M+H⁺].

### N-Benzyl-N-phenethyl-1-phenylpiperidin-4-amine (NCGC00347047-01, XJB14-051)

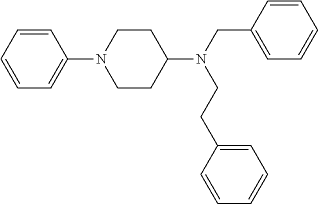 [SMILES: c1ccc(CCN(Cc2ccccc2)C2CCN(c3ccccc3)CC2)cc1]

A mixture of N-benzyl-N-phenethylpiperidin-4-amine (30.0 mg, 0.102 mmol), phenylboronic acid (18.6 mg, 0.153 mmol), DBU (0.031 mL, 0.204 mmol), and copper (II) acetate (37.0 mg, 0.204 mmol) in DMSO (2.00 mL) was heated in μW at 100° C. for 1 h. The mixture was cooled to room temperature and filtered through a cartridge of Tiol to get rid of copper, and purified by HPLC to give the title compound as a TFA salt. ¹H NMR (400 MHz, DMSO-d₆) δ 9.49 (s, 1H), 7.66-7.59 (m, 2H), 7.51 (dd, J=2.13, 4.99 Hz, 3H), 7.37-7.12 (m, 7H), 6.98 (d, J=8.15 Hz, 2H), 6.79 (t, J=7.26 Hz, 1H), 4.63 (dd, J=3.55, 13.73 Hz, 1H), 4.35 (dd, J=7.16, 13.25 Hz, 1H), 3.88 (d, J=11.82 Hz, 2H), 3.72-3.54 (m, 1H), 3.29-3.17 (m, 1H), 3.01 (td, J=5.69, 12.28 Hz, 1H), 2.85-2.68 (m, 4H), 2.22-2.17 (m, 2H), 1.94 (td, J=6.27, 11.29, 11.94 Hz, 2H); LCMS t₁, (Method 1)=4.733 min, m/z 371.2 [M+H⁺].

### 1-(4-(Benzyl(phenethyl)amino)piperidin-1-yl)ethanone (NCGC00347048-01, XJB14-070)

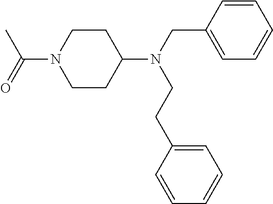 [SMILES: CC(=O)N1CCC(N(CCc2ccccc2)Cc2ccccc2)CC1]

A solution of N-benzyl-N-phenethylpiperidin-4-amine (30.0 mg, 0.102 mmol) in dichloromethane (2.00 mL) was treated at room temperature with acetyl chloride (16.0 mg, 0.204 mmol) and triethylamine (0.043 mL, 0.306 mmol). The reaction mixture was stirred at room temperature for 1 h. The mixture was dried by blowing air, re-dissolved in DMSO, filtered and purified by HPLC to give the title compound as a TFA salt. ¹H NMR (400 MHz, DMSO-d₆) δ 9.48 (s, 1H), 7.65-7.57 (m, 2H), 7.51 (dd, J=2.05, 5.03 Hz, 3H), 7.36-7.20 (m, 3H), 7.20-7.13 (m, 2H), 4.56 (dt, J=4.28, 13.66 Hz, 2H), 4.34 (dt, J=5.74, 12.59 Hz, 1H), 3.96 (d, J=13.35 Hz, 1H), 3.66-3.61 (m, 1H), 3.25-3.13 (m, 1H), 3.14-2.92 (m, 2H), 2.83-2.70 (m, 1H), 2.59-2.51 (m, 1H), 2.17-2.06 (m, 2H), 2.02 (s, 3H), 1.85 (dd, J=7.69, 13.27 Hz, 1H), 1.73-1.62 (m, 1H) (one proton was hidden under water peak); LCMS t₁ (Method 1)=3.776 min, m/z 337.2 [M+H⁺].

### N-Benzyl-1-((2,3-dihydrobenzo[b][1,4]dioxin-6-yl)methyl)-N-phenethylpiperidin-4-amine (NCGC00347050-01, XJB14-076)

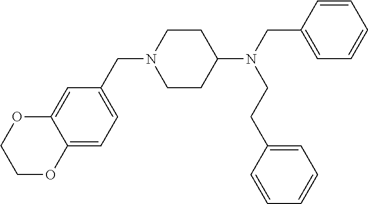 [SMILES: c1ccc(CCN(Cc2ccccc2)C2CCN(Cc3ccc4c(c3)OCCO4)CC2)cc1]

The title compound was prepared according to General Protocol A as a TFA salt. LCMS t₁ (Method 1)=3.747 min, m/z 443.3 [M+H⁺].

### 1-([1,1′-Biphenyl]-4-ylmethyl)-N-benzyl-N-phenethylpiperidin-4-amine (NCGC00347051-01, XJB14-077)

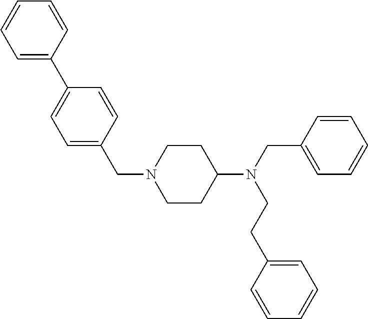 [SMILES: c1ccc(CCN(Cc2ccccc2)C2CCN(Cc3ccc(-c4ccccc4)cc3)CC2)cc1]

The title compound was prepared according to General Protocol A as a TFA salt. LCMS t₁ (Method 1)=4.354 min, m/z 461.3 [M+H⁺].

### N-Benzyl-1-(4-iodobenzyl)-N-phenethylpiperidin-4-amine (NCGC00347052-01, XJB14-074)

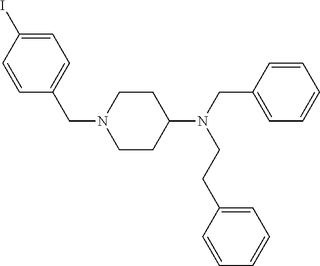 [SMILES: Ic1ccc(CN2CCC(N(CCc3ccccc3)Cc3ccccc3)CC2)cc1]

The title compound was prepared according to General Protocol A as a TFA salt. LCMS t₁ (Method 1)=4.094 min, m/z 511.2 [M+H⁺].

### N-Benzyl-1-(2-methoxybenzyl)-N-phenethylpiperidin-4-amine (NCGC00347053-01, XJB14-075)

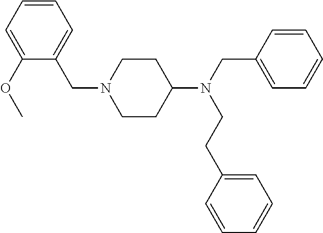 [SMILES: COc1ccccc1CN1CCC(N(CCc2ccccc2)Cc2ccccc2)CC1]

The title compound was prepared according to General Protocol A as a TFA salt. LCMS t₁ (Method 1)=3.783 min, m/z 415.3 [M+H⁺].

### 4-((4-(Benzyl(phenethyl)amino)piperidin-1-yl)methyl)benzonitrile (NCGC00347054-01, XJB14-058)

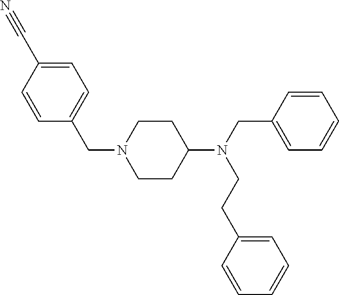 [SMILES: N#Cc1ccc(CN2CCC(N(CCc3ccccc3)Cc3ccccc3)CC2)cc1]

The title compound was prepared according to General Protocol A as a TFA salt. LCMS t₁ (Method 1)=3.823 min, m/z 410.2 [M+H⁺].

### N-Benzyl-1-(4-bromobenzyl)-N-phenethylpiperidin-4-amine (NCGC00347055-01, XJB14-056)

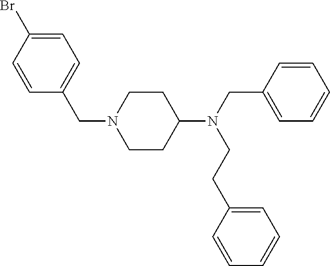 [SMILES: Brc1ccc(CN2CCC(N(CCc3ccccc3)Cc3ccccc3)CC2)cc1]

The title compound was prepared according to General Protocol A as a TFA salt. LCMS t₁ (Method 1)=3.960 min, m/z 463.1 [M+H⁺].

### N-Benzyl-1-(3-methoxybenzyl)-N-phenethylpiperidin-4-amine (NCGC00347056-01, XJB14-057)

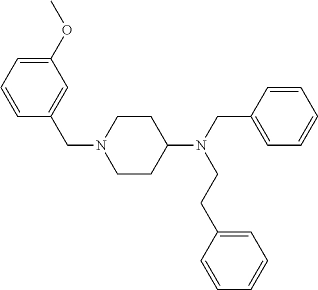 [SMILES: COc1cccc(CN2CCC(N(CCc3ccccc3)Cc3ccccc3)CC2)c1]

The title compound was prepared according to General Protocol A as a TFA salt. LCMS t₁ (Method 1)=3.769 min, m/z 415.2 [M+H⁺].

### N-Benzyl-1-(4-fluorobenzyl)-N-phenethylpiperidin-4-amine (NCGC00347057-01, XJB14-053)

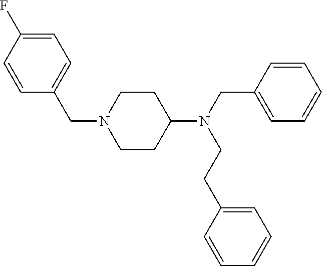 [SMILES: Fc1ccc(CN2CCC(N(CCc3ccccc3)Cc3ccccc3)CC2)cc1]

A solution of N-benzyl-N-phenethylpiperidin-4-amine (30.0 mg, 0.102 mmol) and 4-fluorobenzaldehyde (25.3 mg, 0.204 mmol) in ethanol (2.00 mL) was treated at room temperature with Ts-OH (2.9 mg, 0.015 mmol). The reaction mixture was stirred at room temperature for 10 min, NaCNBH₄ (64.0 mg, 1.02 mmol) was added. The resulting mixture was stirred at room temperature overnight. The mixture was quenched with 1.0 N NaOH aq. solution. The mixture was dried by blowing air, re-dissolved in DMSO, filtered and purified by HPLC to give the title compound as a TFA salt. LCMS t₁ (Method 1)=3.774 min, m/z 403.2 [M+H⁺].

### N-Benzyl-1-(4-methoxybenzyl)-N-phenethylpiperidin-4-amine (NCGC00347058-01, XJB14-054)

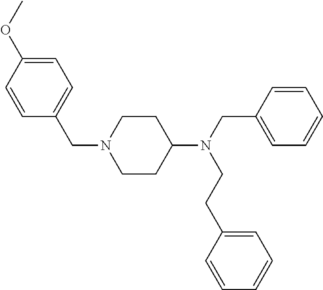 [SMILES: COc1ccc(CN2CCC(N(CCc3ccccc3)Cc3ccccc3)CC2)cc1]

A solution of N-benzyl-N-phenethylpiperidin-4-amine (30.0 mg, 0.102 mmol) and 4-methoxybenzaldehyde (27.7 mg, 0.204 mmol) in ethanol (2.00 mL) was treated at room temperature with Ts-OH (2.9 mg, 0.015 mmol). The reaction mixture was stirred at room temperature for 10 min, NaCNBH₄ (64.0 mg, 1.02 mmol) was added. The resulting mixture was stirred at room temperature overnight. The mixture was quenched with 1.0 N NaOH aq. solution. The mixture was dried by blowing air, re-dissolved in DMSO, filtered and purified by HPLC to give the title compound as a TFA salt. LCMS t₁ (Method 1)=3.874 min, m/z 415.2 [M+H⁺].

### N-Benzyl-N,1-diphenethylpiperidin-4-amine (NCGC00347059-01, XJB14-055)

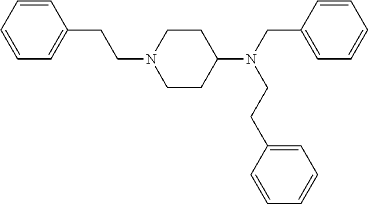 [SMILES: c1ccc(CCN2CCC(N(CCc3ccccc3)Cc3ccccc3)CC2)cc1]

A solution of N-benzyl-N-phenethylpiperidin-4-amine (30.0 mg, 0.102 mmol) and 2-phenylacetaldehyde (24.5 mg, 0.204 mmol) ine (2.00 mL) was treated at room temperature with Ts-OH (2.9 mg, 0.015 mmol). The reaction mixture was stirred at room temperature for 10 min, NaCNBH₄ (64.0 mg, 1.02 mmol) was added. The mixture was quenched with 1.0 N NaOH aq. solution. The mixture was dried by blowing air, re-dissolved in DMSO, filtered and purified by HPLC to give the title compound as a TFA salt. LCMS t₁ (Method 1)=3.865 min, m/z 399.2 [M+H⁺].

### Methyl 4-((4-(benzyl(phenethyl)amino)piperidin-1-yl)methyl)benzoate (NCGC00347206-01, XJB14-078)

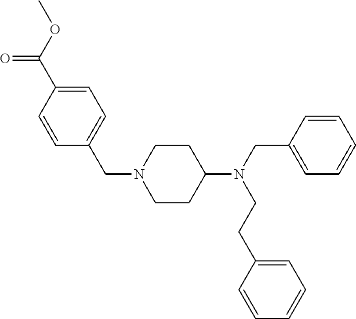 [SMILES: COC(=O)c1ccc(CN2CCC(N(CCc3ccccc3)Cc3ccccc3)CC2)cc1]

The title compound was prepared according to General Protocol A as a TFA salt. LCMS t₁ (Method 1)=3.825 min, m/z 443.3 [M+H⁺].

### N-Benzyl-N-phenethyl-1-propylpiperidin-4-amine (NCGC00347207-01, XJB015-002)

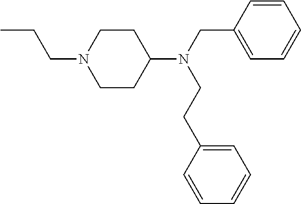 [SMILES: CCCN1CCC(N(CCc2ccccc2)Cc2ccccc2)CC1]

The title compound was prepared according to General Protocol B as a TFA salt. LCMS t₁ (Method 1)=3.436 min, m/z 337.2 [M+H⁺].

### N-Benzyl-1-butyl-N-phenethylpiperidin-4-amine (NCGC00347209-01, XJB015-008)

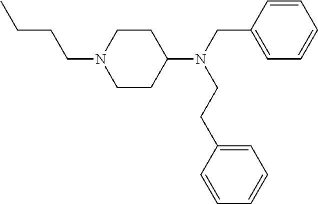 [SMILES: CCCCN1CCC(N(CCc2ccccc2)Cc2ccccc2)CC1]

The title compound was prepared according to General Protocol B as a TFA salt. LCMS t₁ (Method 1)=3.599 min, m/z 351.2 [M+H⁺].

### N-Benzyl-1-ethyl(2,2,2-d₃)-N-phenethylpiperidin-4-amine (XJB015-081)

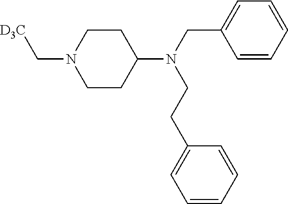 [SMILES: [2H]C([2H])([2H])CN1CCC(N(CCc2ccccc2)Cc2ccccc2)CC1]

The title compound was prepared according to General Protocol B as a TFA salt. LCMS t₁ (Method 1)=3.347 min, m/z 326.3 [M+H⁺].

### N-Benzyl-N-phenethyl-1-(2,2,2-trifluoroethyl)piperidin-4-amine (XJB015-083)

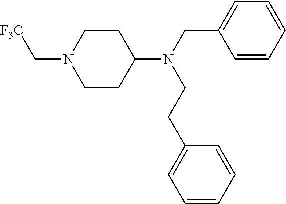 [SMILES: FC(F)(F)CN1CCC(N(CCc2ccccc2)Cc2ccccc2)CC1]

2,2,2-trifluoroethyl trifluoromethanesulfonate (23.7 mg, 0.102 mmol) was added to a stirred mixture of N-benzyl-N-phenethylpiperidin-4-amine (30.0 mg, 0.102 mmol), potassium carbonate (28.2 mg, 0.204 mmol) and Acetonitrile (1.00 mL). The reaction was stirred at room temperature for 5 hours. The mixture was dried by blowing air, re-dissolved in DMSO, filtered and purified by HPLC to give the title compound as a TFA salt. ¹H NMR (400 MHz, DMSO-d₆) δ 9.54 (s, 1H), 7.69-7.07 (m, 10H), 4.67-4.48 (m, 1H), 4.43-4.28 (m, 1H), 3.40-3.10 (m, 4H), 3.09-2.91 (m, 3H), 2.77 (tt, J=6.44, 12.86 Hz, 1H), 2.63-2.50 (m, 1H), 2.49-2.33 (m, 2H), 2.11-2.02 (m, 2H), 1.93-1.79 (m, 2H); LCMS t₁ (Method 1)=4.509 min, m/z 377.2 [M+H⁺].

### N-Benzyl-1-methyl(d₃)-N-phenethylpiperidin-4-amine (XJB015-078, NCGC00351280-01)

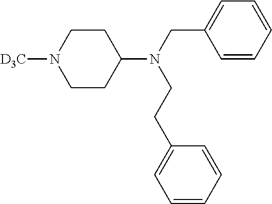 [SMILES: [2H]C([2H])([2H])N1CCC(N(CCc2ccccc2)Cc2ccccc2)CC1]

A solution of N-benzyl-N-phenethylpiperidin-4-amine (50.0 mg, 0.170 mmol) in THF (1.00 mL) and Water (0.500 mL) was treated at room temperature with NaOH (6.8 mg, 0.170 mmol) and MeI-d3 (10.6 μL, 0.170 mmol). The reaction mixture was stirred at 65° C. for 2 h. The mixture was cooled to room temperature, dried by blowing air, re-dissolved in DMSO, filtered and purified by HPLC under basic conditions to give the title compound. LCMS t₁ (Method 1)=3.315 min, m/z 312.2 [M+H⁺].

### 2-(2-(2-(4-((4-Chlorophenyl)(phenyl)methyl)piperazin-1-yl)ethoxy)ethyl)isoindoline-1,3-dione

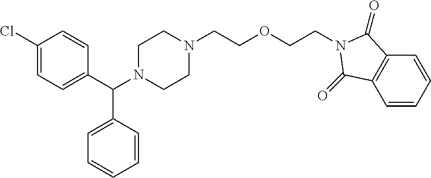 [SMILES: O=C1c2ccccc2C(=O)N1CCOCCN1CCN(C(c2ccccc2)c2ccc(Cl)cc2)CC1]

A solution of 2-(2-(4-((4-chlorophenyl)(phenyl)methyl)piperazin-1-yl)ethoxy)ethanol, 2 HCl (250 mg, 0.667 mmol) in THF (10.0 mL; purchased from TimTec, Newark, Del., USA)) was added Et₃N (0.279 mL, 2.00 mmol) at room temperature. The mixture was stirred for 15 min, then phthalimide (147 mg, 1.000 mmol) and triphenylphosphine (262 mg, 1.00 mmol) were added to the mixture followed by diisopropyl azodicarboxylate (0.130 mL, 0.667 mmol). The reaction mixture was stirred at room temperature for 4 h, after which LCMS analysis showed product formation. Reaction mixture was concentrated to dryness and residue purified by preparative HPLC to give the title compound as the TFA salt. LCMS RT (Method 1)=5.205 min, m/z 505.7 [M+H⁺].

### 2-(2-(4-((4-Chlorophenyl)(phenyl)methyl)piperazin-1-yl)ethoxy)ethanamine

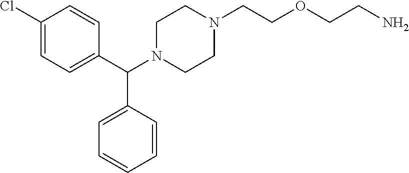 [SMILES: NCCOCCN1CCN(C(c2ccccc2)c2ccc(Cl)cc2)CC1]

Hydrazine (0.181 mL, 5.77 mmol) was added to a solution of 2-(2-(2-(4-((4-chlorophenyl)(phenyl)methyl)piperazin-1-yl)ethoxy)ethyl)isoindoline-1,3-dione (97.0 mg, 0.192 mmol) in EtOH (3.00 mL). The reaction mixture was stirred at 60.0° C. for 3 h, after which LCMS analysis showed completion. The reaction mixture was concentrated under reduced pressure and residue purified by preparative HPLC, to give the title compound as the TFA salt. ¹H NMR (400 MHz, DMSO-d₆) δ ppm 9.42 (s, 1H), 7.72 (s, 4H), 7.46 (d, J=8.4 Hz, 2H), 7.44-7.38 (m, 414), 7.34 (t, J=7.5 Hz, 2H), 7.25 (t, J=7.4 Hz, 1H), 4.53 (s, 1H), 3.73 (d, J=4.8 Hz, 2H), 3.58 (t, J=5.2 Hz, 4H), 3.14 (d, J=11.2 Hz, 2H), 3.04-2.97 (m, 2H), 2.82 (d, J=12.8 Hz, 2H), 2.28 (s, 2H). LCMS RT (Method 1)=3.959 min, m/z 374.7 [M+H⁺].

### tert-Butyl (14-(4-((4-chlorophenyl)(phenyl)methyl)piperazin-1-yl)-3,6,9,12-tetraoxatetradecyl)carbamate

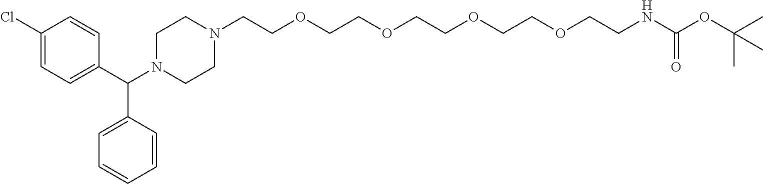 [SMILES: CC(C)(C)OC(=O)NCCOCCOCCOCCOCCN1CCN(C(c2ccccc2)c2ccc(Cl)cc2)CC1]

A solution of 2-(2-(4-((4-chlorophenyl)(phenyl)methyl)piperazin-1-yl)ethoxy)ethanol, 2 HCl (250 mg, 0.558 mmol) in DMF (5.00 mL) was treated with a 60% dispersion in mineral oil of NaH (89.0 mg, 2.23 mmol) at 0° C. The reaction mixture was stirred at 0° C. for 10 min and room temperature for 30 min. To this mixture was added a solution of tert-butyl (2-(2-(2-bromoethoxy)ethoxy)ethyl)carbamate (174 mg, 0.558 mmol) in DMF (1.00 mL) and the resulting mixture allowed to stir overnight. The mixture was quenched with H₂O and extracted with CH₂Cl₂. The organic layer was separated, dried over MgSO₄, filtered and concentrated. Crude residue was purified by preparative HPLC, to give the title compound as the TFA salt. ¹H NMR (400 MHz, CDCl₃) δ ppm 7.45-7.37 (m, 4H), 7.37-7.18 (m, 5H), 4.44 (s, 1H), 3.86 (t, J=4.4 Hz, 2H), 3.63-3.48 (m, 14H), 3.29 (s, 4H), 2.91 (s, 9H), 1.43 (s, 9H). ¹⁹F NMR (376 MHz, CDCl₃) δ ppm −75.78. LCMS RT (Method 1)=5.372 min, m/z 607.7 [M+H⁺].

### 14-(4-((4-Chlorophenyl)(phenyl)methyl)piperazin-1-yl)-3,6,9,12-tetraoxatetradecan-1-amine

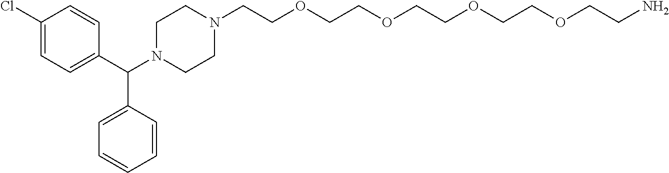 [SMILES: NCCOCCOCCOCCOCCN1CCN(C(c2ccccc2)c2ccc(Cl)cc2)CC1]

A solution of tert-butyl (14-(4-((4-chlorophenyl)(phenyl)methyl)piperazin-1-yl)-3,6,9,12-tetraoxatetradecyl)carbamate (0.217 g, 0.358 mmol) in CH₂Cl₂ (10.0 mL) was treated with trifluoroacetic acid (5.00 mL) at 0° C. The reaction mixture was stirred at 0° C. for 10 min and room temperature for 30 min, after which LCMS analysis showed completion. The reaction mixture was concentrated and the crude residue was purified by preparative HPLC, to give the title compound as the TFA salt. ¹H NMR (400 MHz, CDCl₃) δ ppm 7.95 (s, 2H), 7.51-7.41 (m, 4H), 7.38-7.25 (m, 4H), 4.57 (s, 1H), 3.79 (dd, J=11.2, 6.6 Hz, 4H), 3.70-3.49 (m, 9H), 3.58 (s, 7H), 3.36 (d, J=4.8 Hz, 2H), 3.17 (s, 3H), 3.00 (s, 5H). ¹⁹F NMR (376 MHz, CDCl₃) δ −75.78. LCMS RT (Method 1)=3.916 min, m/z 507.2 [M+H⁺].

### N-(14-(4-((4-Chlorophenyl)(phenyl)methyl)piperazin-1-yl)-3,6,9,12-tetraoxatetradecyl)acetamide

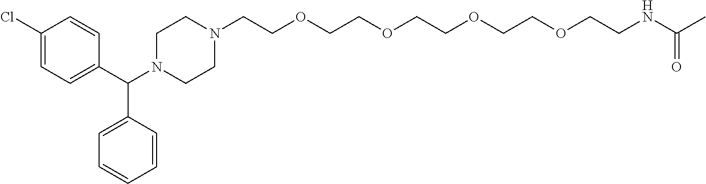 [SMILES: CC(=O)NCCOCCOCCOCCOCCN1CCN(C(c2ccccc2)c2ccc(Cl)cc2)CC1]

A solution of 14-(4-((4-chlorophenyl)(phenyl)methyl)piperazin-1-yl)-3,6,9,12-tetraoxatetradecan-1-amine (14.0 mg, 0.028 mmol) in CH₂Cl₂ (1.00 mL) and Et₃N (0.019 mL, 0.138 mmol) was treated with acetyl chloride (1.97 μL, 0.028 mmol) at 0° C. The reaction mixture was stirred at 0° C. for 10 min and room temperature for 30 min, after which LCMS analysis showed completion. The reaction mixture was concentrated and the crude residue was purified by preparative HPLC, to give the title compound as the TFA salt. ¹H NMR (400 MHz, DMSO-d₆) δ ppm 9.36 (s, 1H), 7.43 (d, J=8.5 Hz, 2H), 7.41-7.35 (m, 5H), 7.32 (t, J=7.5 Hz, 2H), 7.22 (t, J=7.2 Hz, 1H), 4.51 (s, 1H), 3.71 (s, 2H), 3.53 (d, J=4.8 Hz, 4H), 3.46 (hept, J=2.5 Hz, 4H), 3.42 (s, 4H), 3.36 (t, J=5.9 Hz, 2H), 3.28 (s, 4H), 3.17 (tq, J=14.7, 9.0, 7.4 Hz, 4H), 2.79 (d, J=12.7 Hz, 2H), 2.27 (s, 2H), 1.78 (s, 3H). LCMS RT (Method 1)=4.538 min, m/z 549.7 [M+H⁺].

### bis(4-Chlorophenyl)methanol

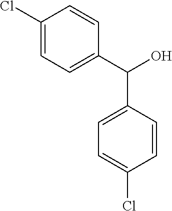 [SMILES: OC(c1ccc(Cl)cc1)c1ccc(Cl)cc1]

A solution of bis(4-chlorophenyl)methanone (27, 3.00 g, 11.9 mmol) in MeOH (15.0 mL) was treated at 0° C. in portions with NaBH₄ (0.678 g, 17.9 mmol). The reaction mixture was stirred at 0° C. for 15 min, allowed to warm to room temperature and stirred for 2 h. The reaction was quenched with ice, diluted with H₂O and extracted with EtOAc. The organic layer was separated, dried over MgSO₄ and concentrated to give the title compound as a white solid, which was used without further purification. ¹H NMR (400 MHz, CDCl₃) δ ppm 7.31 (d, J=8.8 Hz, 4H), 7.28 (d, J=8.7 Hz, 4H), 5.78 (d, J=3.2 Hz, 1H), 2.26 (d, J=3.5 Hz, 1H). LCMS RT (Method 2)=3.733 min, m/z 254.5 [M+H⁺].

### 4,4′-(Chloromethylene)bis(chlorobenzene)

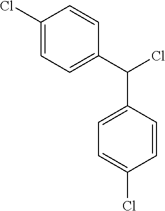 [SMILES: Clc1ccc(C(Cl)c2ccc(Cl)cc2)cc1]

bis(4-Chlorophenyl)methanol (3.00 g, 11.8 mmol) was dissolved in CH₂Cl₂ (10.0 mL), to this was added 3-4 drops of DMF followed by thionyl chloride (2.60 mL, 35.6 mmol). The resulting reaction mixture was allowed to stir at room temperature for 45 min, after which TLC analysis (20% EtOAc in Hex) showed completion. Reaction mixture was concentrated under reduced pressure to afford 28 as a white solid, which was used without further purification. ¹H NMR (400 MHz, CDCl₃) δ ppm 7.42-7.27 (m, 8H), 6.06 (s, 1H). LCMS RT (Method 2)=3.932 min, m/z 272.6 [M+H⁺].

### 1-(bis(4-Chlorophenyl)methyl)piperazine

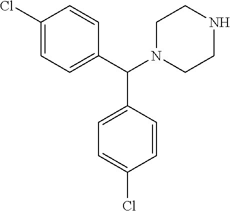 [SMILES: Clc1ccc(C(c2ccc(Cl)cc2)N2CCNCC2)cc1]

A solution of 4,4′-(chloromethylene)bis(chlorobenzene) (80.0 mg, 0.295 mmol) in THF (10.0 mL) was treated with piperazine (38.1 mg, 0.442 mmol) followed by K₂CO₃ (81.0 mg, 0.589 mmol). A catalytic amount of tetrabutylammonium iodide (10.9 mg, 0.029 mmol) was added to the mixture. The reaction mixture was refluxed for 8 h, after which LCMS analysis showed completion. The reaction mixture was concentrated and re-dissolved in EtOAc. The organic layer was washed three times with saturated NaHCO₃ solution, dried over MgSO₄, filtered and concentrated. The crude product was purified by preparative HPLC, to give the title compound as the TFA salt. ¹H NMR (400 MHz, DMSO-d₆) δ ppm 8.50 (s, 2H), 7.43 (d, J=8.7 Hz, 4H), 7.39 (d, J=8.6 Hz, 4H), 4.56 (s, 1H), 3.11 (s, 4H), 2.46 (s, 4H). LCMS RT (Method 1)=4.760 min, m/z 322.7 [M+H⁺].

### 1-(bis(4-Chlorophenyl)methyl)-4-methylpiperazine

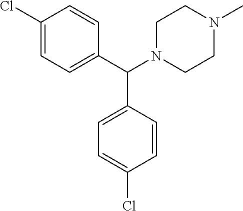 [SMILES: CN1CCN(C(c2ccc(Cl)cc2)c2ccc(Cl)cc2)CC1]

To a stirred solution of 4,4′-(chloromethylene)bis(chlorobenzene) (0.800 g, 2.95 mmol) in THF (10.0 mL) was added K₂CO₃ (0.814 g, 5.89 mmol), 1-methylpiperazine (0.654 mL, 5.89 mmol) and catalytic potassium iodide (73.0 mg, 0.442 mmol). The reaction was heated to 100° C. for 48 h. The reaction mixture was partitioned between EtOAc and H₂O, the layers separated and the organic phase washed with brine, dried over MgSO₄, filtered and concentrated. Crude mixture was purified by flash column chromatography: silica gel with a gradient of 0-5% MeOH in CH₂Cl₂ to afford the title compound as a free-base oil, which was then mixed in a 1:1 ratio with oxalic acid to form the oxalate salt. ¹H NMR (400 MHz, DMSO-d₆) δ ppm 7.41 (d, J=8.6 Hz, 4H), 7.34 (d, J=8.5 Hz, 4H), 4.33 (s, 1H), 2.32 (s, 4H), 2.27 (s, 4H), 2.14 (s, 3H). LCMS RT (Method 1)=4.843 min, m/z 336.9 [M+H⁺].

### 1-(bis(4-Chlorophenyl)methyl)-4-ethylpiperazine

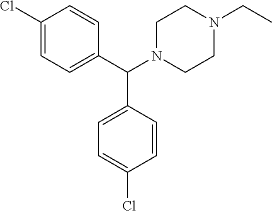 [SMILES: CCN1CCN(C(c2ccc(Cl)cc2)c2ccc(Cl)cc2)CC1]

A solution of 4,4′-(chloromethylene)bis(chlorobenzene) (160 mg, 0.589 mmol) in THF (10.0 mL) was treated with 1-ethylpiperazine (101 mg, 0.884 mmol) followed by K₂CO₃ (163 mg, 1.18 mmol). A catalytic amount of tetrabutylammonium iodide (21.8 mg, 0.059 mmol) was added, and the resulting reaction mixture was heated to 100° C. for 48 hours. The reaction mixture was partitioned between EtOAc and H₂O, the layers separated and the organic phase washed with brine, dried over MgSO₄, filtered and concentrated. Crude mixture was purified by flash column chromatography: silica gel with a gradient of 0-5% MeOH in CH₂Cl₂ to afford the title compound as a free-base oil, which was then mixed in a 1:1 ratio with oxalic acid to form the oxalate salt. ¹H NMR (400 MHz, DMSO-d₆) δ ppm 7.44 (d, J=8.8 Hz, 4H), 7.40 (d, J=8.8 Hz, 4H), 4.57 (s, 1H), 3.11-3.02 (m, 2H), 2.80 (s, 8H), 2.24 (s, 2H), 1.17 (t, J=7.2 Hz, 3H). LCMS RT (Method 1)=5.029 min, m/z 350.7 [M+H⁺].

### 1-(bis(4-Chlorophenyl)methyl)piperidine

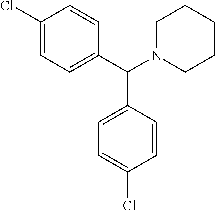 [SMILES: Clc1ccc(C(c2ccc(Cl)cc2)N2CCCCC2)cc1]

A solution of 4,4′-(chloromethylene)bis(chlorobenzene) (80.0 mg, 0.295 mmol) in THF (10.0 mL) was treated with piperidine (37.6 mg, 0.442 mmol) followed by K₂CO₃ (81.0 mg, 0.589 mmol) and a catalytic amount of tetrabutylammonium iodide (10.9 mg, 0.029 mmol). The resulting reaction mixture was refluxed for 8 h, after which LCMS analysis showed product formation. The reaction mixture was concentrated and then taken up in EtOAc. The organic layer was washed three times with saturated NaHCO₃ solution, brine, dried over MgSO₄, filtered and concentrated to an oil. The crude product was purified by preparative HPLC, to afford the title compound as the TFA salt. ¹H NMR (400 MHz, DMSO-d₆) δ ppm 9.96 (s, 1H), 7.67 (d, J=8.3 Hz, 3H), 7.58 (d, J=8.1 Hz, 3H), 7.43-7.33 (m, 2H), 5.71 (d, J=9.3 Hz, 1H), 3.24-3.16 (m, 2H), 2.94-2.86 (m, 2H), 1.89-1.80 (m, 2H), 1.71-1.66 (m, 3H), 1.45-1.36 (m, 1H). LCMS RT (Method 1)=4.584 min, m/z 321.7 [M+H⁺].

### 4-(bis(4-Chlorophenyl)methyl)morpholine

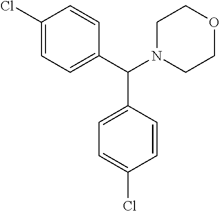 [SMILES: Clc1ccc(C(c2ccc(Cl)cc2)N2CCOCC2)cc1]

A solution of 4,4′-(chloromethylene)bis(chlorobenzene) (50.0 mg, 0.184 mmol) in acetonitrile (6.00 mL) was treated with morpholine (48.1 mg, 0.552 mmol). The reaction mixture was refluxed for 3 h. The mixture was concentrated and purified by preparative HPLC to afford the title compound as the TFA salt. ¹H NMR (400 MHz, DMSO-d₆) δ ppm 7.46-7.41 (m, 4H), 7.40-7.35 (m, 4H), 4.36 (s, 1H), 3.59 (s, 4H), 3.11 (s, 1H), 2.26 (s, 4H). LCMS RT (Method 1)=4.728 min, m/z 323.3 [M+H⁺].

### 2-(2-(4-(bis(4-Chlorophenyl)methyl)piperazin-1-yl)ethoxy)ethanol

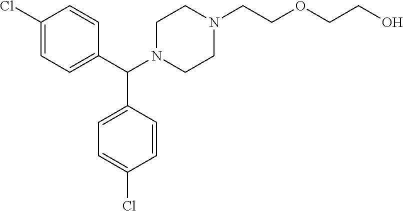 [SMILES: OCCOCCN1CCN(C(c2ccc(Cl)cc2)c2ccc(Cl)cc2)CC1]

A solution of 1-(bis(4-chlorophenyl)methyl)piperazine (100 mg, 0.311 mmol) in H₂O (1.50 mL) was treated with K₂CO₃ (86.0 mg, 0.623 mmol) and tetrabutylammonium chloride (87.0 mg, 0.311 mmol). The resulting mixture was stirred at room temperature for 15 min, then 2-(2-chloroethoxy)ethanol (38.8 mg, 0.311 mmol) in acetonitrile (1.50 mL) was added to the mixture. The resulting reaction mixture was heated to 100° C. for 2 h, after which LCMS analysis showed completion. The reaction mixture was diluted with EtOAc and washed with H₂O and brine. The organic layer was separated, dried over MgSO₄, filtered and concentrated. Residue was purified by preparative HPLC to afford the title compound as the TFA salt. ¹H NMR (400 MHz, DMSO-d₆) δ ppm 9.34 (s, 1H), 7.47-7.36 (m, 8H), 4.58 (s, 1H), 3.72 (t, J=4.9 Hz, 2H), 3.55-3.49 (m, 4H), 3.49-3.42 (m, 4H), 3.13 (d, J=11.5 Hz, 3H), 2.80 (d, J=12.9 Hz, 2H), 2.27 (t, J=12.2 Hz, 2H). LCMS RT (Method 1)=4.716 min, m/z 410.4 [M+H⁺].

### 14-(4-(bis(4-Chlorophenyl)methyl)piperazin-1-yl)-3,6,9,12-tetraoxatetradecan-1-amine

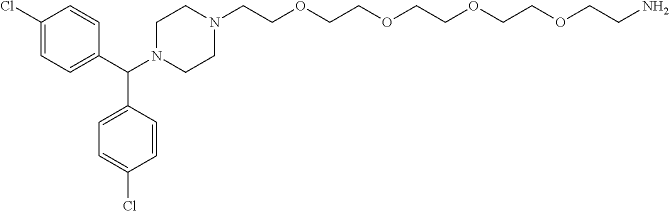 [SMILES: NCCOCCOCCOCCOCCN1CCN(C(c2ccc(Cl)cc2)c2ccc(Cl)cc2)CC1]

A solution of 2-(2-(4-(bis(4-chlorophenyl)methyl)piperazin-1-yl)ethoxy)ethanol (250 mg, 0.518 mmol) in DMF (5.00 mL) was treated with a 60% dispersion in mineral oil of NaH (83.0 mg, 2.07 mmol) at 0° C. The reaction mixture was stirred at 0° C. for 10 min and room temperature for 30 min. To this mixture was then added a solution of tert-butyl (2-(2-(2-bromoethoxy)ethoxy)ethyl)carbamate (162 mg, 0.518 mmol) in DMF (1.00 mL) and the resulting reaction mixture allowed to stir overnight. The mixture was quenched with H₂O and extracted with CH₂Cl₂. The organic layers were separated, dried over MgSO₄, filtered and concentrated. The residue was taken up in CH₂Cl₂ (10.0 mL) and treated with trifluoroacetic acid (5.00 mL) at 0° C. The reaction mixture was stirred at 0° C. for 10 min and room temperature for 30 min. The reaction mixture was concentrated and purified by preparative HPLC to afford the title compound as the TFA salt. ¹H NMR (400 MHz, DMSO-d₆) δ ppm 9.54 (s, 1H), 7.77 (s, 3H), 7.47-7.37 (m, 7H), 4.58 (s, 1H), 3.72 (t, J=4.9 Hz, 2H), 3.61-3.43 (m, 14H), 3.45-3.40 (m, 2H), 3.30 (d, J=5.1 Hz, 2H), 3.13 (d, J=10.5 Hz, 2H), 2.97 (h, J=5.6 Hz, 2H), 2.80 (d, J=12.8 Hz, 2H), 2.28 (t, J=12.4 Hz, 2H). LCMS RT (Method 1)=4.208 min, m/z 541.5 [M+H⁺].

### 1-((4-Bromophenyl)(phenyl)methyl)-4-ethylpiperazine

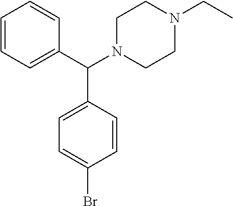 [SMILES: CCN1CCN(C(c2ccccc2)c2ccc(Br)cc2)CC1]

To a solution of 1-((4-bromophenyl)(phenyl)methyl)piperazine (50.0 mg, 0.151 mmol) in MeOH (2.00 mL) was added acetaldehyde (33.2 mg, 0.755 mmol), NaBH₃CN (28.5 mg, 0.453 mmol) and acetic acid (0.026 mL, 0.453 mmol). The reaction was stirred at room temperature overnight. The reaction was quenched with 1 N NaOH solution. The mixture was dried by blowing air, re-dissolved in DMSO, filtered and purified by preparative HPLC to afford the title compound as the TFA salt. LCMS RT (Method 1)=4.594 min, m/z 360.3 [M+H⁺].

### 1-((4-Bromophenyl)(4-chlorophenyl)methyl)-4-ethylpiperazine

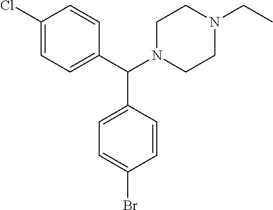 [SMILES: CCN1CCN(C(c2ccc(Cl)cc2)c2ccc(Br)cc2)CC1]

To a solution of 1-((4-bromophenyl)(4-chlorophenyl)methyl)piperazine (50.0 mg, 0.151 mmol) in MeOH (2.00 mL) was added acetaldehyde (33.2 mg, 0.755 mmol), NaBH₃CN (28.5 mg, 0.453 mmol) and acetic acid (0.026 mL, 0.453 mmol). The reaction was stirred at room temperature overnight. The reaction was quenched with 1 N NaOH solution. The mixture was dried by blowing air, re-dissolved in DMSO, filtered and purified by preparative HPLC to afford the title compound as the TFA salt. ¹H NMR (400 MHz, DMSO-d₆) δ ppm 9.16 (s, 1H), 7.54 (d, J=8.4 Hz, 2H), 7.47-7.33 (m, 6H), 4.59 (d, J=6.6 Hz, 1H), 3.41 (d, J=12.3 Hz, 2H), 3.13 (s, 2H), 3.03 (q, J=11.3, 10.8 Hz, 2H), 2.83 (d, J=12.8 Hz, 2H), 2.20 (t, J=12.2 Hz, 2H), 1.18 (t, J=7.2 Hz, 3H). LCMS RT (Method 1)=4.950 min, m/z 394.7 [M+H^{÷}].

### 1-Methyl-N-phenylpiperidin-4-amine

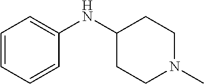

A solution of aniline (500 mg, 5.37 mmol) and 1-methylpiperidin-4-one (1.24 mL, 10.7 mmol) in MeOH (10.0 mL) was treated at room temperature with acetic acid (0.615 mL, 10.7 mmol). After stirring for 10 min, NaBH₃CN (1.69 g, 26.8 mmol) was added, and the resulting reaction mixture allowed to stir overnight. A 2 N NaOH solution was then added to adjust the pH to 10. The mixture was extracted with CH₂Cl₂, and the combined organic layers were dried over MgSO₄, filtered and concentrated. The crude product was purified by flash column chromatography: silica gel with 0-100% EtOAc in hexanes to get rid of the first peak. Then 20% MeOH in CH₂Cl₂ to afford the title compound as a white solid. LCMS RT (Method 2)=2.171 min, m/z 191.3 [M+H⁺].

### N-(4-Chlorophenyl)-1-methylpiperidin-4-amine

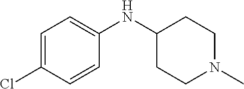

A solution of 4-chloroaniline (500 mg, 3.92 mmol) and 1-methylpiperidin-4-one (0.905 mL, 7.84 mmol) in MeOH (10.0 mL) was treated at room temperature with acetic acid (0.449 mL, 7.84 mmol). After stirring for 10 min, NaBH₃CN (1.69 g, 26.8 mmol) was added, and the resulting reaction mixture allowed to stir overnight. A 2 N NaOH solution was then added to adjust the pH to 10. The mixture was extracted with CH₂Cl₂, and the combined organic layers were dried over MgSO₄, filtered and concentrated. The crude product was purified by flash column chromatography: silica gel with 0-100% EtOAc in hexanes to get rid of the first peak. Then 20% MeOH in CH₂Cl₂ to afford the title compound as a as a yellow oil. LCMS RT (Method 2)=2.345 min, m/z 225.1 [M+H⁺].

### 1-Methyl-N,N-diphenylpiperidin-4-amine

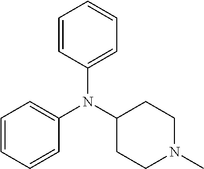 [SMILES: CN1CCC(N(c2ccccc2)c2ccccc2)CC1]

A mixture of 1-methyl-N-phenylpiperidin-4-amine (141 mg, 0.741 mmol), iodobenzene (0.165 mL, 1.48 mmol), Pd(OAc)₂ (16.6 mg, 0.074 mmol), BINAP (50.8 mg, 0.082 mmol), and potassium tert-butoxide (104 mg, 0.926 mmol) (1.0 M solution in THF, 0.167 mL) in toluene (0.200 mL) was stirred at 110° C. for 4 h. The reaction was cooled to room temperature and treated with Si-Thiol. The mixture was dried by blowing air, re-dissolved in DMSO, filtered and purified by preparative HPLC to give the title compound as the TFA salt. LCMS RT (Method 1)=4.218 min, m/z 267.2 [M+H⁺].

### N-(4-(tert-Butoxy)phenyl)-1-methyl-N-phenylpiperidin-4-amine

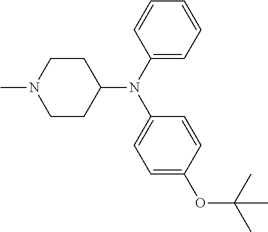 [SMILES: CN1CCC(N(c2ccccc2)c2ccc(OC(C)(C)C)cc2)CC1]

A mixture of N-(4-chlorophenyl)-1-methylpiperidin-4-amine (30.0 mg, 0.133 mmol), iodobenzene (0.030 mL, 0.267 mmol), Pd(OAc)₂ (3.00 mg, 0.013 mmol), BINAP (9.14 mg, 0.015 mmol), and potassium tert-butoxide (18.7 mg, 0.167 mmol) (0.167 mmol, 1.0 M solution in THF, 0.167 mL) in toluene (0.200 mL) was stirred at 110° C. for 4 h. The reaction was cooled to room temperature and treated with Si-Thiol. The mixture was dried by blowing air, re-dissolved in DMSO, filtered and purified by preparative HPLC to give the final product as a TFA salt. LCMS RT (Method 1)=4.656 min, m/z 339.1 [M+H⁺].

### 2-Bromo-9-chloro-9H-fluorene

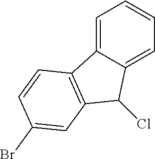 [SMILES: ClC1c2ccccc2-c2ccc(Br)cc21]

A solution of 2-bromo-9H-fluoren-9-one (1.00 g, 3.86 mmol) in MeOH (5.00 mL) was treated at 0° C. with NaBH₄ (0.219 g, 5.79 mmol). The resulting reaction mixture was stirred at room temperature overnight. The reaction was quenched with ice water and extracted into EtOAc. The organic layer was separated, dried over MgSO₄, filtered and concentrated to give the intermediate alcohol 2-bromo-9H-fluoren-9-ol as white solid (0.920 g, 91%). A solution of this intermediate alcohol 2-bromo-9H-fluoren-9-ol (500 mg, 1.91 mmol) in concentrated HCl (10.0 mL, 329 mmol) was treated at with calcium chloride (298 mg, 2.68 mmol). The resulting reaction mixture was refluxed for 4 h. The reaction was cooled to room temperature and extracted into EtOAc. The organic layer was separated and dried over MgSO₄, filtered and concentrated to give the title compound as a white solid, which was used without further purification. LCMS RT (Method 2)=3.974 min, m/z 280.6 [M+H⁺].

### 1-(2-Bromo-9H-fluoren-9-yl)-4-ethylpiperazine

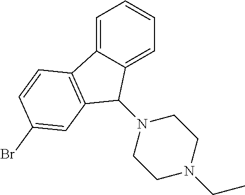 [SMILES: CCN1CCN(C2c3ccccc3-c3ccc(Br)cc32)CC1]

A solution of 2-bromo-9-chloro-9H-fluorene (100 mg, 0.358 mmol) in THF (10.0 mL) was treated at with 1-ethylpiperazine (0.068 mL, 0.537 mmol) followed by K₂CO₃ (99.0 mg, 0.715 mmol), and a catalytic amount of tetrabutylammonium iodide (13.2 mg, 0.036 mmol). The resulting reaction mixture was refluxed for 8 h, after which LCMS analysis showed product formation. The reaction mixture was concentrated and the residue taken up in EtOAc, washed with H₂O, brine, dried over MgSO₄, filtered and concentrated. Crude residue was purified by flash column chromatography: silica gel with a gradient of 0-20% MeOH in CH₂Cl₂ to give the title compound as a colorless oil which was converted into the oxalic acid salt. LCMS RT (Method 1)=4.598 min, m/z 358.2 [M+H⁺].

### Example 2

This example demonstrates the potent reduction of HCV RNA levels by chlorocyclizine hydrochloride (“CCZ”) in a cell culture-derived HCV assay, in accordance with an embodiment of the invention.

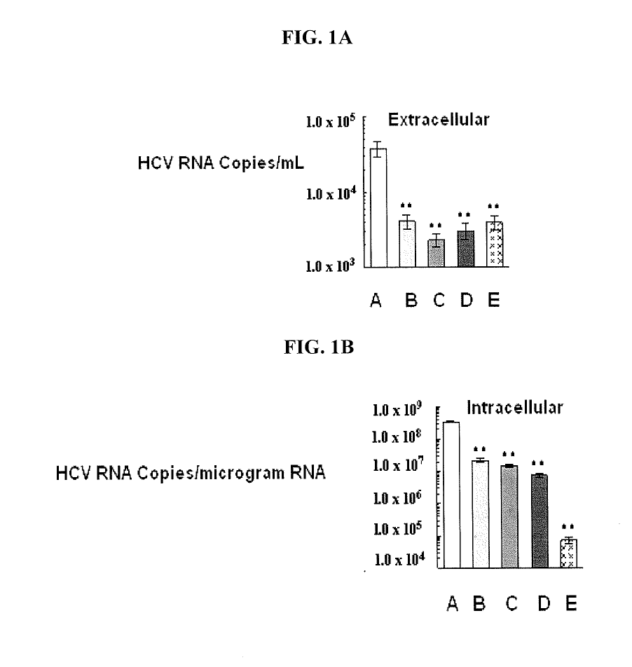

Huh 7.5.1 cells were seeded in 12-well plates (10⁵ cells/well) and cultured overnight. HCVcc was used to infect the cells with the treatment of compounds at 10 μM. Virus-containing medium was removed after 4 h incubation and compound treatment was added back followed by incubation for additional 48 h. Intracellular and extracellular viral RNA levels were evaluated by quantitative real-time PCR. The results are illustrated in FIG. 1 and are the means of three replicates ±SEM. Asterisks (**P<0.0001) indicate statistically significant reduction of the compound-treated results from the DMSO-treated results by Student's t test. Cyclosporin A at 10 μM was used as positive control.

Cell Culture-derived HCV (HCVcc, genotype 2a, JFH-1 strain) system provides direct evidence of anti-HCV activity of the compounds. The results illustrated in FIG. 1 show that the extracellular and intracellular viral RNA levels were reduced with the treatment of racemic, (R)- and (S)-CCZ.

### Example 3

This example demonstrates that CCZ targets early stages in the HCV life cycle, but not entry or replication stages, in accordance with an embodiment of the invention.

To investigate the stages of virus life cycle where compounds of the invention act on, HCV single-cycle infection assay, HCV subgenomic replicon assays and HCV pseudoparticle (HCVpp) assays were performed with the treatment of racemic, (R)- and (S)-CCZ at 10 μM.

A. Huh 7.5.1 cells seeded in 96-well plates (10⁴ cells/well) were cultured overnight. The cells were inoculated with the infectious HCVsc together with the tested compounds. Luciferase activity of the cells was measured 48 h after the compound treatment.

B. HCV subgenomic replicon assays. HCV replicon (GT 1b and 2a) cells were plated into 96-well plate (10⁴ cells/well) and incubated overnight. The cells were treated with tested compounds. Luciferase activity of the cells was measured 48 h after the compound treatment. In transient replicon assay, Huh 7.5.1 cells seeded in 96-well plates (10⁴ cells/well) were cultured overnight. Then the cells were transiently transfected with the replicon mRNA with DMRIE-C for 4 h. After removing the transfection reagent, the cells were incubated with DMEM culture medium containing 10 μM of each compound for 48 h. Luciferase activity was measured.

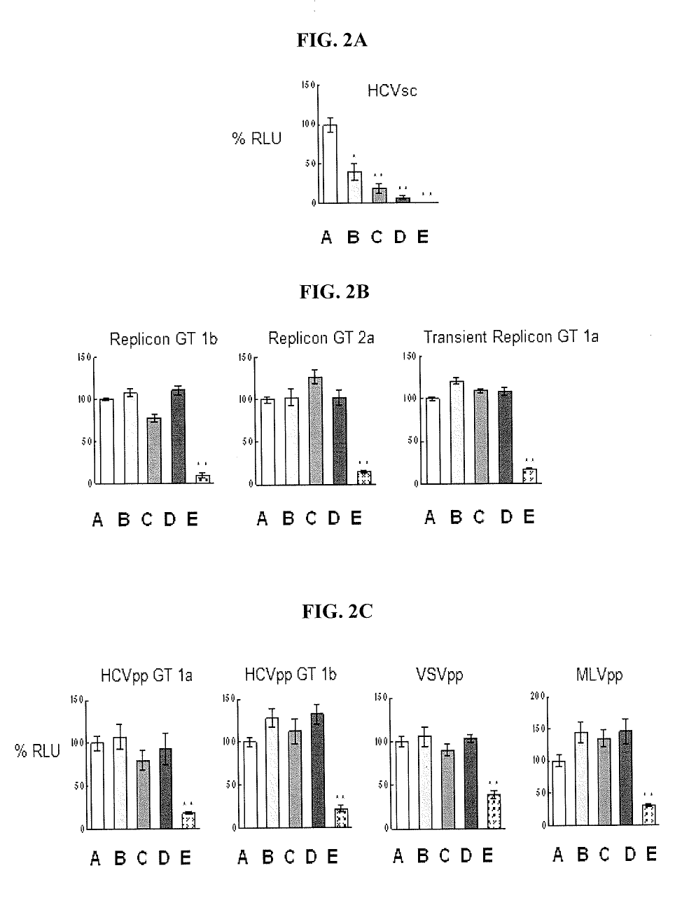

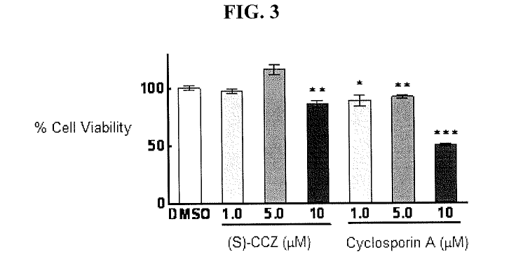

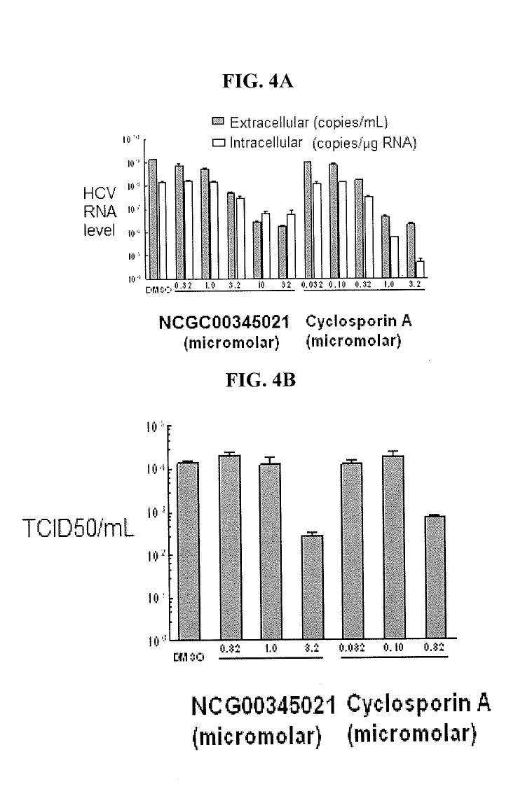

C. HCVpp assays. Huh 7.5.1 cells were seeded in 96-well plates (10⁴ cells/well) and cultured overnight. Then the cells were treated with 10 uM of the compounds together with infection of HCVpp GT 1a, 1b, VSVpp and MLVpp for 4 h. The cells were then washed and cultured for 48 h followed by a luciferase assay to detect the HCV entry. The results shown are the means of at least five replicates ±SEM. Asterisks (**P<0.0001 and *P<0.0005) indicate the statistical significance of more than 50% reduction of the compound-treated results from the DMSO-treated results by Student's t test. Cyclosporin A and rottlerin at 10 μM were used as positive controls. FIG. 2A illustrates the results of the HCV single-cycle infection assay. FIG. 2B illustrates the results of the HCV subgenomic replicon assays. FIG. 2C illustrates the results of the HCV pseudoparticle (“HCVpp)” assays.

In HCV single-cycle infection assay (Masaki, T. et al., J. Virology, 2010, 84: 5824-5835), the single round infectious HCV defective particle (HCVsc, genotype 2a) were used to infect Huh 7.5.1 cells. The HCVsc can infect and replicate but does not assemble new virions, thus this assay detects compounds with inhibitory activity to HCV life cycle events prior to assembly. Shown in FIG. 2A, racemic, (R) and (S)-CCZ showed significant inhibitory activities in the HCVsc infection level, and this confirmed that chlorocyclizine HCl inhibits HCV early-stage infection. HCV subgenomic replicon assays evaluate whether compounds target viral RNA replication. Racemic, (R)- and (S)-CCZ were used to treat replicon genotype (GT) 1b and 2a cell lines and did not show much inhibitory effect. Besides, transient transfection was performed with replicon GT 1a in Hub7.5.1 cells before compound treatment, and no inhibition was observed. Therefore, results from these HCV subgenomic replicon assays indicate that replication is not the target of these compounds of the invention, wherein m=n=0 and o=1, in HCV life cycle. HCVpp (GT 1a and 1b) are defective retroviral particles that display HCV envelope glycoproteins, and they are used to assess the effect of compound treatment on viral entry. VSVpp and MLVpp were also tested in the entry assay as control viruses for virus selectivity. None of racemic, (R) and (S)-CCZ showed any inhibitory activities in HCVpp assays, suggesting that inhibition of viral entry is not the mechanism of anti-HCV action of CCZ analogues.

### Example 4

This example demonstrates the synergistic antiviral effect of CCZ with current anti-HVC drugs, in accordance with an embodiment of the invention.

Combination of ribavirin and peginterferon α (IFN-α) has been the standard of care to treat chronic HCV infection for many years. Direct-acting antivirals, such as telaprevir and daclatasvir, were recently approved for therapy of hepatitis C. The combination of (S)-CCZ with these different classes of anti-HCV drugs is described in this example. HCV-Luc assay in parallel with ATPlite assay was performed in the presence of various concentrations of (S)-CCZ in combination with various concentrations of each drug. Using the MacSynergy II program based on Bliss independence model, three-dimensional surface plots were generated and log volume of synergism was calculated for each combination. The results were also analyzed with the CalcuSyn program, in which the combination indices were calculated. The results are set forth in Table 1. The antiviral effect of (S)-CCZ is highly synergistic with ribavirin, interferon-α, telaprevir (NS3/4A inhibitor), daclatasvir (NS5A inhibitor), cyclosporin A (CSA), boceprevir, and sofosbubir without significant cytotoxicity, supporting its use in combination therapy with these drugs.

TABLE 1

| | Param- | | | | | | | |
|---|---|---|---|---|---|---|---|---|
| Program | eter | Ribavirin | IFN-α | Telaprevir | Boceprevir | Sofosbuvir | Daclataivir | CSA |
| Mac | Log | +++ | +++ | +++ | +++ | +++ | +++ | ++ |
| Synergy | volume | | | | | | | |
| CalcuSyn | CI | 0.630 ± 0.106 | 0.609 ± 0.128 | 0.426 ± 0.138 | 0.691 ± 0.114 | 0.362 ± 0.075 | 0.427 ± 0.142 | 0.727 ± 0.187 |
| | value | | | | | | | |
| | Synergy | +++ | +++ | +++ | +++ | +++ | +++ | +++ |
| | volume | | | | | | | |

The observed synergistic effects suggest that (S)-CCZ inhibits HCV infection through a different mechanism from any one of these drugs. The mechanism of action of ribavirin and IFN-α is mediated through host antiviral response. Telaprevir is NS3/4A protease inhibitor and daclatasvir inhibits HCV NS5A (Lin, K. et al., Antimicrobial Agents and Chemotherapy, 2006, 50: 1813-1822; Gao, M. et al., Nature, 2010, 465: 96-U108). Cyclosporin A targets virus RNA replication and 2′-C-methylcytidine is a NS5B polymerase inhibitor (Gao et al., ibid, De Francesco, R. et al., Nature, 2005, 436: 953-960). The synergistic effect of (S)-CCZ with these reagents suggests that its mechanism of action is novel and unique. This makes CCZ an attractive agent for development with a possibly unique mechanism and lower probability of selecting resistant virus strains during treatment.

### Example 5

This example demonstrates the lack of long-term in vitro cytotoxicity of chlorcyclizine hydrochloride.

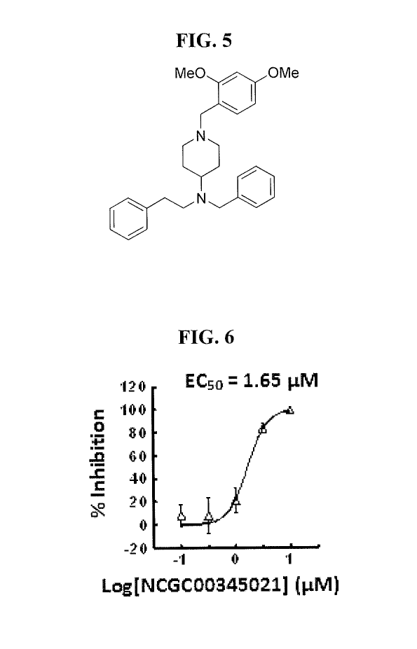

Huh 7.5.1 cells seeded in 6-well plates (2×10⁶ cells/well) were cultured overnight before treatment with the test compounds. In the presence of the compound, cells were passaged every 3 days for 7 passages, and plated to 96-well plates 3 days prior to ATPlite assay. The results are shown in FIG. 3 and are the means of eight replicates ±SEM. Asterisks ((*P<0.05 **P<0.005 and ***P<0.0001) indicate statistical significance of compound-treated results from the DMSO-treated results by Student's t test. Cyclosporin A was tested as a positive control. The cell viability as a function of concentration is illustrated in FIG. 3, demonstrating the lack of long-term in vitro cytotoxicity of chlorcyclizine hydrochloride.

### Example 6

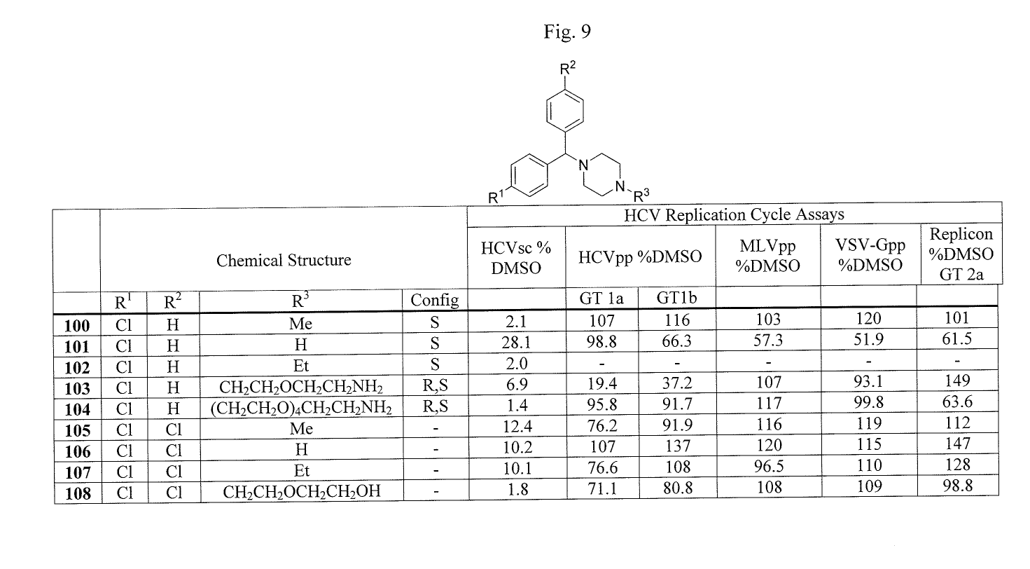

This example demonstrates that compounds of formula (I), for example, NCGC00345021, target the late stage of the HCV life cycle, in accordance with an embodiment of the invention. The structure of NCGC00345021 is shown in FIG. 5.

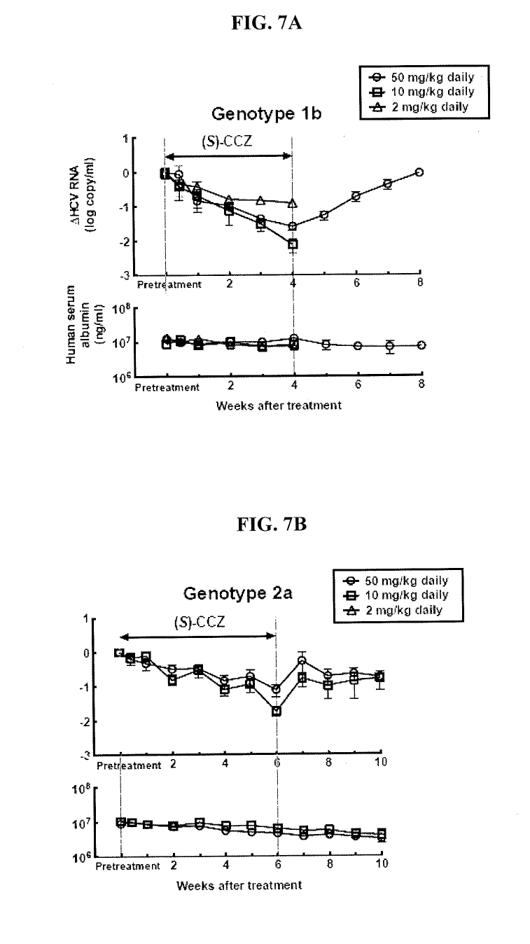

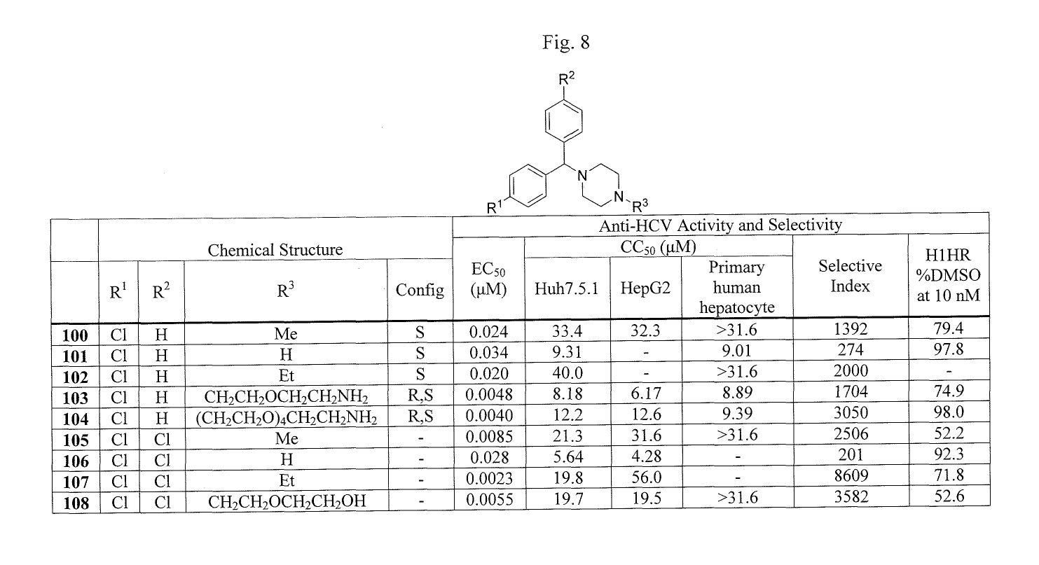

Cell Culture-derived HCV (HCVcc, genotype 2a, JFH-1 strain) system provides direct evidence of anti-HCV activity of the compounds. Determination of both extracellular and intracellular HCV levels can help evaluate whether the compounds interfere early-stage or late-stage infection. If a compound inhibits late-stage infection (virus assembly or secretion), a more dramatic reduction of extracellular virus RNA level will be observed. Cyclosporin A was tested in parallel to serve as control compound targeting early-stage HCV infection. As shown in FIG. 4A, the extracellular and intracellular viral RNA levels were dramatically reduced with the treatment of NCGC00345021 and cyclosporin A in a dose-dependent manner. At the highest concentration, cyclosporin A caused approximately 4-log fold reduction in intracellular RNA copies, while the extracellular level reduced less than S-log fold. On the contrary, NCGC00345021 led to only about 1-log fold decrease in intracellular RNA copies when causing 3-log fold reduction in extracellular RNA level. Clearly, when the concentration increased, NCGC00345021 led to a more dramatic reduction in extracellular RNA copies. When medium containing extracellular viruses were used to re-infect nave Huh 7.5.1 cells, NCGC00345021 led to a dose-dependent reduction in TCID50 values, confirming its effect on extracellular RNA copies (FIG. 4B). The results from the HCVcc assay followed with TCID50 determination suggest that NCGC00345021 and analogs thereof target late stages in HCV life cycle.

To further confirm that compounds in accordance with an embodiment of the invention target late stage of virus life cycle, HCV single-cycle infection assay, HCV subgenomic replicon assays and HCV pseudoparticle (HCVpp) assays were performed with the treatment of NCGC00345021 at 10 μM. In HCV single-cycle infection assay (Masaki, t. et al., J. Virology, 2010, 84: 5824-5835), the single round infectious HCV defective particle (HCVsc, genotype 2a) were used to infect Huh 7.5.1 cells. The HCVsc can infect and replicate but does not assemble new virions, thus this assay detects compounds with inhibitory activity to HCV life cycle events prior to assembly. Shown in Table 2, NCGC00345021 showed no significant inhibitory activities in the HCVsc infection level. HCV subgenomic replicon assays evaluate whether compounds target viral RNA replication. Transient transfection assay with GT 2a replicon RNA in Hub7.5.1 cells showed modest inhibition of viral replication by. However, NCGC00345021 did not show any inhibitory effect of HCV replication in genotype 2a replicon cell line. HCVpp (GT 1a and 1b) are defective retroviral particles that display HCV envelope glycoproteins, and they are used to assess the effect of compound treatment on viral entry. VSVpp was also tested in the entry assay as control viruses for virus selectivity. NCGC00345021 showed low inhibitory activity in HCVpp GT 1a level and no inhibition on VSVpp. Taking NCGC00345021 led to more than 90% inhibition in HCV-Luc infection at 10 nM, the lack of more than 50% inhibitory effects of NCGC00345021 at 10 μM in these other assays suggests that NCGC00345021 and analogs thereof target more of a late stage of viral life cycle.

In HCV single-cycle infection assay, Huh7.5.1 cells seeded in 96-well plates (10⁴ cells/well) were cultured overnight. The cells were inoculated with the infectious HCVsc together with the tested compounds. Luciferase activity of the cells was measured 48 h after the compound treatment. In transient replicon assay, Huh7.5.1 cells seeded in 96-well plates (10⁴ cells/well) were cultured overnight. Then the cells were transiently transfected with the replicon RNA transcript with DMRIE-C for 4 h. After removing the transfection reagent, the cells were incubated with DMEM culture medium containing 10 μM of each compound for 48 h. Luciferase activity was measured. In HCV subgenomic replicon assay with HCV replicon (GT 2a) cells, cells were plated into 96-well plate (10⁴ cells/well) and incubated overnight. The cells were treated with tested compounds. Luciferase activity was measured 48 h after the compound treatment. In HCVpp assays, Huh 7.5.1 cells were seeded in 96-well plates (10⁴ cells/well) and cultured overnight. Then the cells were treated with 10 μM of the compounds together with infection of HCVpp GT 1a and VSVpp for 4 h. The cells were then washed and cultured for 48 h followed by a luciferase assay to detect the HCV entry. The results shown in Table 2 are the means of five replicates ±SEM.

TABLE 2
Activity of NCGC00345021 in HCV life cycle assays.

| HCV | | HCV subgenomic replicon | | | |
|---|---|---|---|---|---|
| life | | Transient | GT 2a | HCVpp | |
| cycle | | GT | cell | GT | |
| assay | HCVsc | 2a | line | 1a | VSVpp |
| % RLU | 104 ± 9.95 | 139 ± 16.4 | 54.4 ± 2.96 | 66.3 ± 9.49 | 103 ± 6.73 |
| at | | | | | |
| 10 μM | | | | | |

### Example 7

This example demonstrates the inhibition of Dengue virus infection by a compound of formula (I), in accordance with an embodiment of the invention.

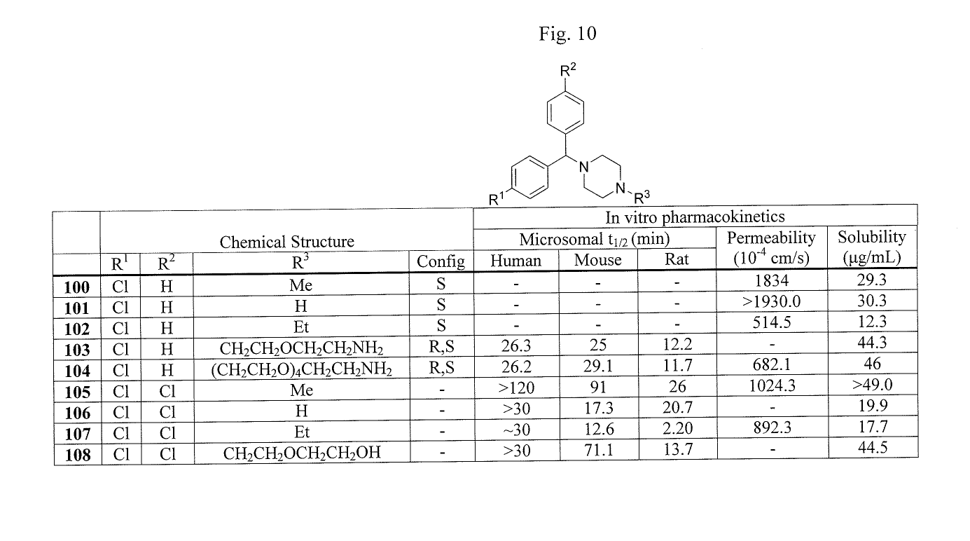

HCV belongs to the flavivirus genus. To explore the possible antiviral activity of NCGC00345021 and analogs thereof on other flaviviruses, NCGC00345021 was tested in Dengue Reporter Virus Particles (RVPs) reproducibility assay. Huh 7.5.1 cells seeded in 96-well plates (10⁴ cells/well) were cultured overnight. Dengue RVP (Integral Molecular) was added to Huh 7.5.1 cells in the presence of increasing concentrations of tested compound (NCGC00345021). Dengue RVP reproducibility was measured by luciferase signal 48 h after treatment. As shown in FIG. 6, a dose-dependent inhibition of Dengue RVP reproducibility was observed with the treatment by NCGC00345021. The results are means of three replicates ±SEM. This result suggests compounds of formula (I) may have a broad anti-viral activities, at least against the Flavividae family of viruses.

### Example 8

This example demonstrates the anti-HCV activity and the cell toxicity of compounds of formula (I), wherein X is N, Y is CH, m=n=0, and o=1. EC₅₀ was generated using the HCV-Luc infection assay and IC₅₀ using the ATPLite assay. The results are set forth in Tables 3-5. The configuration at the carbon marked with an asterisk is indicated in Tables 2 and 3.

TABLE 3

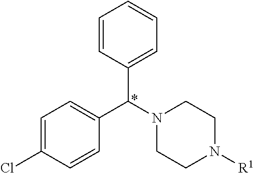

| | | | | Selective |
|---|---|---|---|---|
| R¹ | Configuration | EC₅₀ (μM) | CC₅₀ (μM) | Index |
| Me | R,S | 0.044 ± 0.011 | 49.8 ± 17.2 | 1132 |
| Me | S | 0.024 ± 0.009 | 33.4 ± 2.4 | 1392 |
| Me | R | 0.020 ± 0.005 | 37.5 ± 4.2 | 1875 |
| H | R,S | 0.035 ± 0.013 | 10.4 ± 0.2 | 297 |
| H | S | 0.034 ± 0.012 | 9.31 ± 0.04 | 0274 |
| H | R | 0.032 ± 0.022 | 12.5 ± 1.4 | 391 |
| Et | S | 0.020 ± 0.002 | 40.0 ± 1.1 | 2000 |
| Et | R | 0.0099 ± 0.0054 | 37.9 ± 3.3 | 3828 |
| n-Pr | S | 0.032 ± 0.018 | 39.7 ± 0.9 | 1241 |
| n-Pr | R | 0.024 ± 0.005 | 48.5 ± 1.1 | 2021 |
| i-Pr | S | 0.013 ± 0.004 | 32.4 ± 3.8 | 2492 |
| n-Bu | S | 0.042 ± 0.023 | 31.1 ± 4.2 | 740 |
| n-Bu | R | 0.195 ± 0.084 | 40.8 ± 0.4 | 309 |
| 2-methyl-1-propyl | S | 0.102 ± 0.018 | 51.7 ± 1.4 | 507 |
| 2-methyl-1-propyl | R | 0.232 ± 0.061 | 58.2 ± 7.2 | 251 |
| Cyclopentyl | S | 0.019 ± 0.006 | 30.4 ± 2.8 | 1600 |
| Cyclohexyl | S | 0.177 ± 0.037 | 12.3 ± 0.3 | 69 |
| Acetyl | S | 22.0 ± 3.2 | 91.3 ± 1.0 | 4 |
| 2-4-dimethoxybenzyl | S | 0.456 ± 0.235 | 38.4 ± 4.2 | 84 |
| CD₃ | S | 0.063 ± 0.025 | 77.9 ± 3.1 | 1237 |
| CD₃ | R | 0.040 ± 0.017 | 46.7 ± 0.4 | 1168 |
| CD₃CH₂ | S | 0.036 ± 0.015 | 71.3 ± 11.4 | 1981 |
| CD₃CH₂ | R | 0.035 ± 0.008 | 81.5 ± 0.5 | 2329 |
| CF₃CH₂ | S | >31.6 | >100 | ND |
| CF₃CH₂ | R | >31.6 | >100 | ND |
| CH₂CH₂OCH₂COOH | R,S | >31.6 | >100 | ND |
| CH₂CH₂OCH₂CONH₂ | R,S | 0.103 ± 0.052 | >100 | >910 |
| 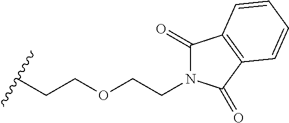 [SMILES: *CCOCCN1C(=O)c2ccccc2C1=O] | R,S | 1.54 ± 0.57 | >100 | >65 |
| CH₂CH₂OCH₂CH₂OH | R,S | 0.032 ± 0.011 | 42.6 ± 1.8 | 1331 |
| CH₂CH₂OCH₂CH₂NH₂ | R,S | 0.0048 ± 0.0011 | 8.18 | 1704 |
| (CH₂CH₂O)₄CH₂CH₂NH₂ | R,S | 0.0040 ± 0.0022 | 12.2 ± 0.8 | 3050 |
| (CH₂CH₂O)₄CH₂CH₂NHC(═O)Me | R,S | 0.170 ± 0.022 | >100 | >588 |
| (CH₂CH₂O)₄(CH₂)₂NHC(═O)Ot-Bu | R,S | 0.199 ± 0.030 | 19.0 ± 4.0 | 95 |

TABLE 4

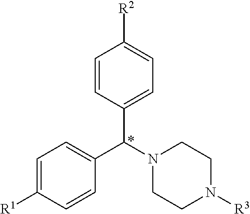

| | | | | | | Selective |
|---|---|---|---|---|---|---|
| R¹ | R² | R³ | Config. | EC₅₀ (μM) | CC₅₀ (μM) | Index |
| Cl | H | Me | R,S | 0.044 ± 0.011 | 49.8 ± 17.2 | 1132 |
| H | H | Me | — | 1.14 ± 0.37 | >100 | >88 |
| Cl | Cl | Me | — | 0.0085 ± 0.0029 | 21.3 ± 2.3 | 2506 |
| Cl | H | H | R,S | 0.035 ± 0.013 | 10.4 ± 2 | 297 |
| Cl | Cl | H | — | 0.028 ± 0.005 | 5.64 ± 0.80 | 201 |
| Br | H | H | — | 0.063 ± 0.014 | 7.93 ± 0.83 | 126 |
| Cl | Br | H | R,S | 0.010 ± 0.004 | 2.26 ± 0.29 | 226 |
| Cl | H | Et | S | 0.020 ± 0.002 | 40.0 ± 1.1 | 2000 |
| Cl | Cl | Et | — | 0.0023 ± 0.0009 | 19.8 ± 1.9 | 8609 |
| Br | H | Et | R,S | 0.0070 ± 0.0004 | 35.2 ± 1.4 | 5029 |
| Cl | Br | Et | R,S | 0.0040 ± 0.0016 | 31.7 ± 3.4 | 5425 |
| Cl | H | CH₂CH₂OCH₂CH₂OH | R,S | 0.032 ± 0.011 | 42.6 ± 1.8 | 1331 |
| Cl | Cl | CH₂CH₂OCH₂CH₂OH | — | 0.0055 ± 0.0022 | 19.7 ± 2.4 | 3582 |
| Cl | H | (CH₂CH₂O)₄CH₂CH₂NH₂ | R,S | 0.0040 ± 0.0022 | 12.2 ± 0.8 | 3050 |
| Cl | Cl | (CH₂CH₂O)₄CH₂CH₂NH₂ | — | 0.014 ± 0.001 | 4.43 ± 0.12 | 316 |

TABLE 5

| | | | | Selective |
|---|---|---|---|---|
| Compound | Config. | EC₅₀ (μM) | CC₅₀ (μM) | Index |
| 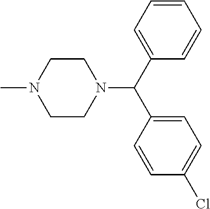 [SMILES: CN1CCN(C(c2ccccc2)c2ccc(Cl)cc2)CC1] | R,S | 0.044 ± 0.011 | 49.8 ± 17.2 | 1132 |
| 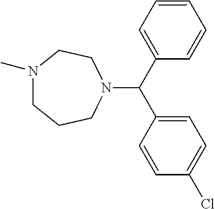 [SMILES: CN1CCCN(C(c2ccccc2)c2ccc(Cl)cc2)CC1] | R,S | 0.057 ± 0.008 | 12.8 ± 0.1 | 225 |
| 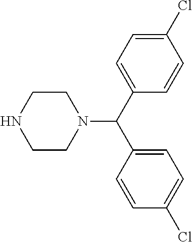 [SMILES: Clc1ccc(C(c2ccc(Cl)cc2)N2CCNCC2)cc1] | — | 0.028 ± 0.005 | 5.64 ± 0.80 | 201 |
| 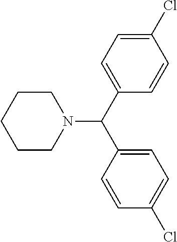 [SMILES: Clc1ccc(C(c2ccc(Cl)cc2)N2CCCCC2)cc1] | — | 17.4 ± 2.8 | 69.0 ± 0.9 | 4 |
| 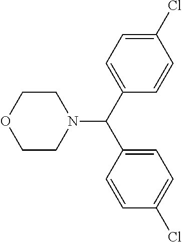 [SMILES: Clc1ccc(C(c2ccc(Cl)cc2)N2CCOCC2)cc1] | — | 29.7 ± 0.1 | 62.1 ± 2.8 | 2 |
| 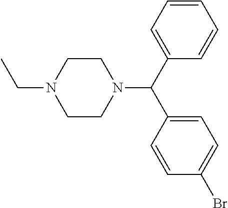 [SMILES: CCN1CCN(C(c2ccccc2)c2ccc(Br)cc2)CC1] | R,S | 0.0070 ± 0.0004 | 35.2 ± 1.4 | 5029 |
| 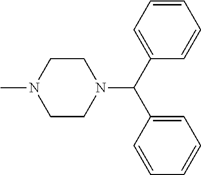 [SMILES: CN1CCN(C(c2ccccc2)c2ccccc2)CC1] | — | 1.14 ± 0.37 | >100 | >88 |
| 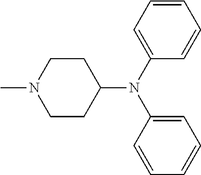 [SMILES: CN1CCC(N(c2ccccc2)c2ccccc2)CC1] | — | 2.72 ± 1.13 | 56.8 ± 8.2 | 21 |
| 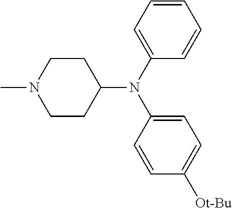 [SMILES: CN1CCC(N(c2ccccc2)c2ccc(OC(C)(C)C)cc2)CC1] | R,S | 0.354 ± 0.097 | 78.7 ± 0.6 | 222 |
| 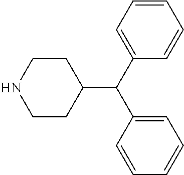 [SMILES: c1ccc(C(c2ccccc2)C2CCNCC2)cc1] | — | 0.072 ± 0.002 | 32.5 ± 0.1 | 451 |
| 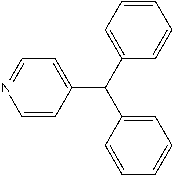 [SMILES: c1ccc(C(c2ccccc2)c2ccncc2)cc1] | — | >31.5 | 65.1 ± 2.1 | <2 |
| 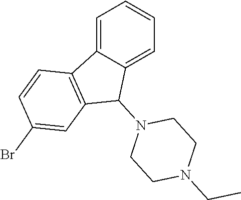 [SMILES: CCN1CCN(C2c3ccccc3-c3ccc(Br)cc32)CC1] | R,S | 1.68 ± 0.45 | 53.1 ± 1.3 | 32 |

### Example 9

This example demonstrates the anti-HCV activity and the cell toxicity of compounds of formula (I), wherein X is N, Y is CH, m=n=0, and o=2, and wherein X is CH, Y is N, m=n=0, and o=1. EC₅₀ was generated using the HCV-Luc infection assay and TC₅₀ using the ATPLite assay.

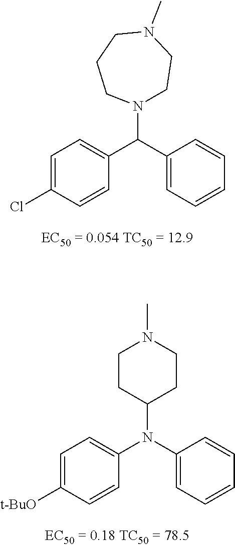 [SMILES: CN1CCCN(C(c2ccccc2)c2ccc(Cl)cc2)CC1 ; CN1CCC(N(c2ccccc2)c2ccc(OC(C)(C)C)cc2)CC1]

### Example 10

This example demonstrates the anti-HCV activity and the cell toxicity of compounds, of formula (I), wherein X is CH, Y is N, Ar¹ and Ar² are both phenyl, m=1, n=2, and o=1. EC₅₀ was generated using the HCV-Luc infection assay and TC₅₀ using the ATPLite assay. The results are set forth in Table 6.

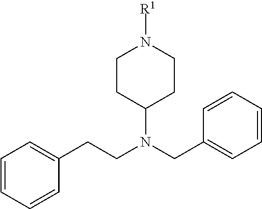

TABLE 6

| R¹ | EC₅₀ (μM) | TC₅₀ (μM) |
|---|---|---|
| H | 0.12 | 23.5 |
| Benzoyl | 24.8 | 94.0 |
| Me | 0.92 | 41.1 |
| Et | 0.039 | 35.3 |
| Phenylsulfonyl | >32.6 | >100 |
| Benzyl | 0.32 | 30.9 |
| Cyclopentyl | 0.48 | 15 |
| 4-Methylbenzyl | 0.74 | 40.3 |
| 4-Chlorobenzyl | 9.24 | >100 |
| Isobutyl | 4.76 | 37.6 |
| Isopropyl | 0.94 | 37.6 |
| 4-Trifluoromethylbenzyl | >10 | >100 |
| Cyclohexyl | 1.5 | 33.8 |
| Phenyl | >31.6 | >100 |
| Acetyl | >31.6 | 74.5 |
| 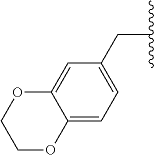 [SMILES: *Cc1ccc2c(c1)OCCO2] | 2.78 | 36.6 |
| 4-Phenylbenzyl | 12.2 | >100 |
| 4-Iodobenzyl | 2.98 | >100 |
| 2-Methoxybenzyl | 0.64 | 15.2 |
| 4-Cyanobenzyl | 5.86 | 100 |
| 4-Bromobenzyl | 4.33 | >100 |
| 3-Methoxybenzyl | 1.57 | 45.2 |
| 4-Fluorobenzyl | 9.6 | 56.3 |
| 4-Methoxybenzyl | 4.35 | 27.8 |
| 2-Phenylethyl | 16.2 | >100 |
| 4-Methoxycarbonylbenzyl | 14 | >100 |
| Propyl | 0.37 | 38.2 |
| Butyl | 1.55 | 17.9 |
| CD₃CH₂— | 2.37 | 50.8 |
| CF₃CH₂— | >31.6 | >100 |
| CD₃— | 7.95 | 100 |

### Example 11

This example demonstrates inhibition of HCV genotype 1b and 2a infections in vivo by chlorcyclizine HCl without clear evidence of drug resistance.

(S)-Chlorcyclizine HCl was tested in Alb-UPA/SCID chimeric mouse model infected with HCV genotype 1b and 2a respectively (Meuleman, P. et al., Nature, 2008, Antiviral Research, 80: 231-238; Turrini, P. et al., Transplantation Proceedings, 2006, 38: 1181-1184). Alb-UPA/SCID mice were engrafted with primary human hepatocytes and then infected with HCV serum samples of genotype 1b or 2a. The mice were monitored for serum HCV RNA and human albumin for 4-6 weeks before treatment. The serum HCV RNA levels were stable with little fluctuations during the weeks before infection, and the pretreatment HCV RNA values were determined by averaging HCV RNA levels of week −2, −1 and 0 before initiation of treatment.

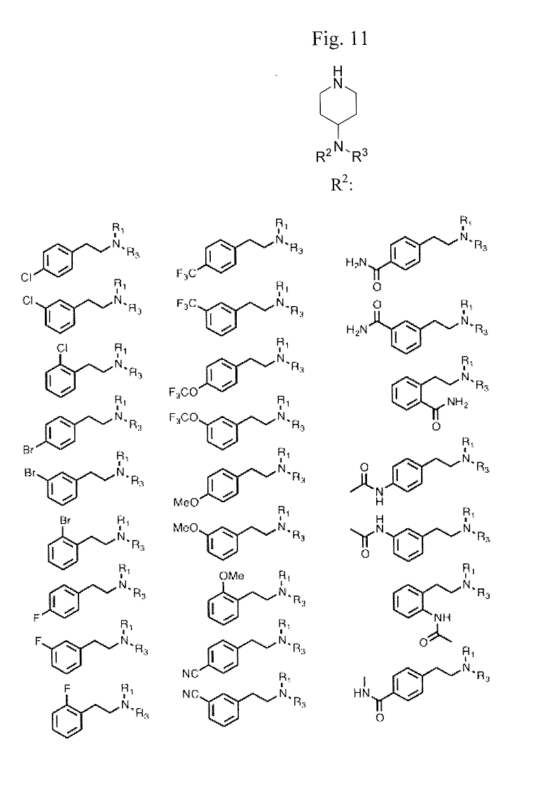

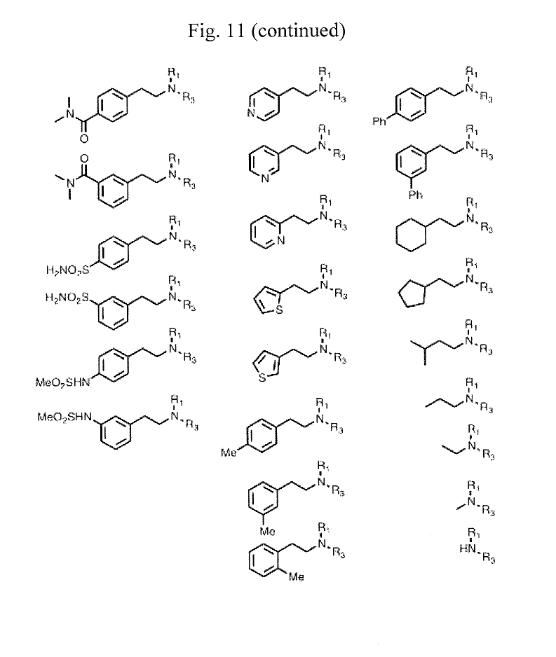

As shown in FIGS. 7A and 7B, the doses of 50 mg/kg and 10 mg/kg daily led to time-dependent reduction of HCV titer from pretreatment baseline in mice with genotype 1b and 2a infection (2-log and 1.5-log, respectively). Dose as low as 2 mg/kg daily also caused significant decrease of genotype 1b virus titer (approximately 1-log). A rebound of virus titer after stopping of treatment was observed in both genotype infections. However, HCV titers continued to decline during the treatment period without rebound, suggesting absence of emergence of drug-resistant virus. This antiviral profile is similar to that of mice treated with IFN-α. FIG. 7A shows changes in the genotype 1b HCV titers from pretreatment baseline over a period of 8 weeks with 4-week (S)-CCZ treatment and 4-week of follow-up without treatment (only in the group received 50 mg/kg dose) in HCV-infected chimeric mice. The results shown are the means of mice in each group ±SEM (n=5 in the 50 mg/kg daily group; n=4 in the 10 mg/kg daily group; n=5 in the 2 mg/kg daily group); FIG. 7B shows changes in the genotype 2a HCV titers from pretreatment baseline over a period of 10 weeks with 6-week (S)-CCZ treatment and 4-week of follow-up without treatment (in both groups) in HCV-infected chimeric mice. The results shown are the means of mice in each group ±SEM (n=8 in the 50 mg/kg daily group; n=5 in the 10 mg/kg daily group).

### Example 12

This example demonstrates the anti-HCV activity and pharmacokinetics profiles of embodiments of the invention.

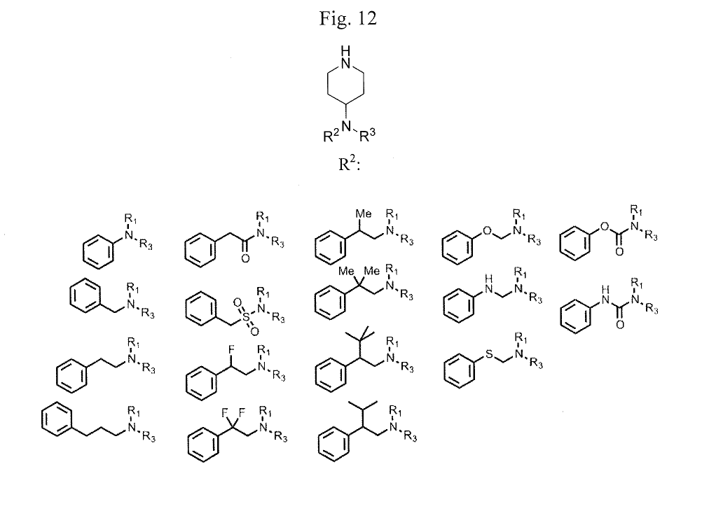

Lead compounds were selected based on anti-HCV activity, selectivity and structure diversity. The structures of the compounds are as set forth in Tables 7-9. The cytotoxicity of the compounds was further evaluated in HepG2 cells and primary human hepatocytes. The EC50 values and cytotoxicity data are set forth in FIG. 8. All compounds showed less than 1.5-fold difference in CC₅₀ values in these two cell types as that in Huh7.5.1 cells, except that compound 107 showed a CC₅₀ in HepG2 cells that is approximately 3-fold higher than that of Huh7.5.1 cells. The H1-histamine receptor (H1HR) binding activity of chosen leads were evaluated with 101 and 100 as the negative and positive controls. As shown in Table 6, lead compounds with R³ as H or a long chain showed less than 10% inhibition (compounds 106 and 104). Meanwhile when R³ is Me, Et or a middle length chain, the H1HR inhibitory effect that is comparable to that of 100 were observed (compounds 105, 102, 107, 103 and 108).

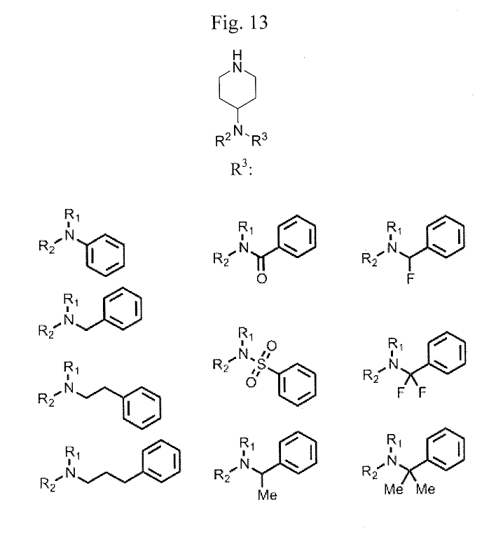

HCV replication cycle assays were carried out to study the target stage of the CCZ analogues in HCV replication cycle. The results are set forth in FIG. 9. The lead compounds exhibited potent inhibition in HCV single-cycle assay, in which single-round infectious HCV (HCVsc) infected hepatocytes but did not assemble into new virions (Table 4). The activity suggests that the CCZ analogues inhibit the early steps in the HCV replication cycle prior to assembly. The analogues were tested in HCV pseudoparticle (HCVpp) assay and HCV subgenomic replicon assay, which detect whether the compounds target the pseudoparticle entry and viral RNA replication, respectively. HCVpp assay applies defective retroviral particles that harbor HCV envelope glycoproteins to detect viral entry inhibition. No significant inhibitory effect was observed in HCVpp (genotype 1a and 1b) assay with the lead compounds, except for 103 possible due to cytotoxicity (Table 4). To address viral specificity in the entry process VSV-Gpp and MLVpp were also tested as control, in which no inhibitory effect was detected. All lead compounds showed more than 60% of DMSO group in both genotype 1b and 2a HCV replicon cell lines, indicating RNA replication is not the target of these analogues.

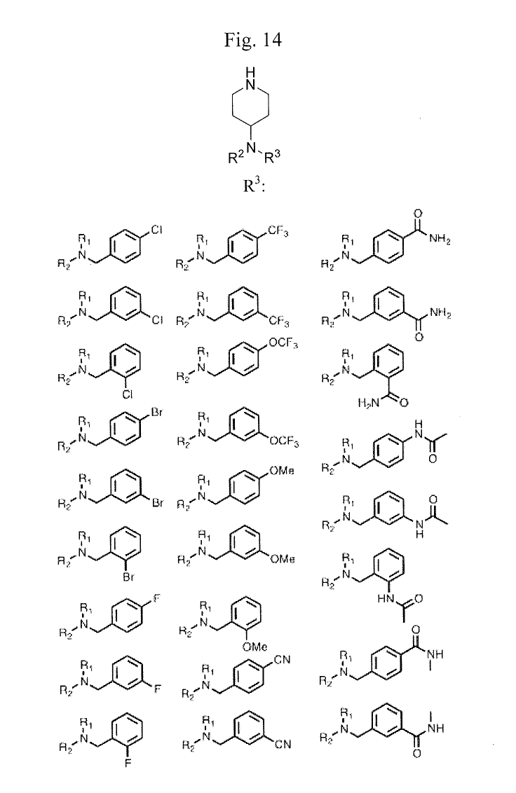

The in vitro ADME properties of chosen lead compounds were measured in microsomal stability assay with human, mouse and rat microsomes. The results, along with permeability and solubility data, are set forth in FIG. 10. All compounds were in the form of TFA salts except for 101. Compound 106, 105, 107, and 108 all showed preferable human microsomal stability (t_{1/2}>30 min). In vivo pharmacokinetics and tissue distribution of 108 were measured in mice after a single dose of 10 mg/kg through intraperitoneal (i.p.) route. The half time in liver was 4.6 h, which is consistent with that determined in human liver microsomal half time. Preferable liver distribution was observed, evidenced by the liver/plasma AU_{Clast} ratio of 11. To detect potential hepatotoxicity effect, the alanine transaminase level in mouse serum was measured. Only 1 mouse at 1 h post-dosing showed slightly elevated ALT level and the rest of the samples are all below 80 U/L. There is no clear correlation between the ALT level and compound liver concentration. Overall, no clear hepatotoxicity was detected in this condition.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A compound of formula (I) 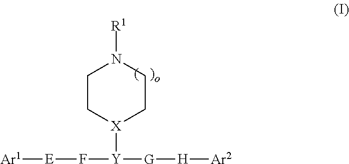
wherein R¹ is selected from hydrogen, C₁-C₁₀ alkyl, C₃-C₁₀ cycloalkyl, C₃-C₁₀ cycloalkyl C₁-C₁₀ alkyl, C₆ aryl, C₆-C₁₀ aryl C₁-C₁₀ alkyl, C₆-C₁₀ aryl C₃-C₁₀ cycloalkyl, heteroaryl, heterocyclyl, C₆₋₁₀ arylsulfonyl, C₆₋₁₀ arylcarbonyl, C₁₋₁₀ alkylcarbonyl, —(CH₂)ₓA(CH₂)_{y}B, and —(CH₂CH₂O)ₚ(CH₂CH₂)_{q}D, wherein the alkyl, aryl, or heteroaryl part of R¹ is optionally substituted with one or more substituents selected from deuterium, halo, C₁-C₁₀ alkyl, C₆-C₁₀ aryl, trifluoromethyl, C₁-C₁₀ alkoxy, cyano, alkylenedioxy, C₁-C₁₀ alkylcarbonyl, and C₁-C₁₀ alkoxycarbonyl,
Ar¹ and Ar² are different and are independently selected from C₆-C₁₀ aryl, heteroaryl, and heterocyclyl, wherein the aryl, heteroaryl, and heterocyclyl are optionally substituted with one or more substituents selected from halo, C₁-C₁₀ alkyl, C₆-C₁₀ aryl, trifluoromethyl, C₁-C₁₀ alkylcarbonyl, and C₁-C₁₀ alkoxycarbonyl,
A is O, S, or N,
x and y are independently 1-4, inclusive,
B is OR⁴,
wherein R⁴ is C₁-C₁₀ alkyl, C₃-C₁₀ cycloalkyl, or C₆-C₁₀ aryl,
D is NR⁸R⁹,
R⁸ and R⁹ are independently selected from hydrogen, COR¹⁰, and COOR¹¹,
R¹⁰ and R¹¹ are hydrogen or C₁-C₁₀ alkyl,
p and q are independently 1-4, inclusive,
E is absent or is (CR¹³R¹⁴)ₘ, NH, or S,
F is absent or is —SO₂—,
G is absent or is (CR¹⁷CR¹⁸)ᵣ,
H is absent or is —SO₂—,
m, n, and r are independently 0, 1, 2, 3, or 4,
o is 0 or 1,
X and Y are independently CH or N,
R¹³, R¹⁴, R¹⁷, and R¹⁸ are independently hydrogen, F, or methyl,
or pharmaceutically acceptable salts, stereoisomers, and mixtures comprising stereoisomers thereof,
with the provisos that (i) when E, F, G, and H are all absent, o is 1, X is N, Y is CH, and R¹ is hydrogen, methyl, ethyl, or isopropyl, the compound is a single enantiomer at the carbon bearing Ar¹ and Ar², and (ii) when E, F, G, and H are all absent, o is 1, X is CH and Y is N, R¹ is hydrogen, methyl, or ethyl.

2. The compound, salt, stereoisomers, and mixtures comprising stereoisomers of claim 1, wherein X is CH and Y is N and 0 is 1.

3. (canceled)

4. The compound, salt, stereoisomers, and mixtures comprising stereoisomers of claim 1, wherein E is (CR¹³R¹⁴)ₘ, F is absent, and m is 2, and H is absent and r is 1.

5. (canceled)

6. (canceled)

7. The compound, salt, stereoisomers, and mixtures comprising stereoisomers of claim 1, wherein R¹ is selected from C₁-C₁₀ alkyl, C₃-C₁₀ cycloalkyl, and C₃-C₁₀ cycloalkyl C₁-C₁₀ alkyl.

8. The compound, salt, stereoisomers, and mixtures comprising stereoisomers of claim 1, wherein R¹ is selected from hydrogen, cyclopentyl, sec-butyl, isopropyl, cyclohexyl, n-propyl, n-butyl, benzoyl, methyl, ethyl, trideuteromethyl, 2,2,2-trideuteroethyl, 2,2,2-trifluoroethyl, phenylsulfonyl, and benzyl.

9. The compound, salt, stereoisomers, and mixtures comprising stereoisomers of claim 1, wherein R¹ is selected from C₆ aryl and C₆-C₁₀ aryl C₁-C₁₀ alkyl, wherein the aryl is optionally substituted with one or more substituents selected from halo, cyano, alkylenedioxy, C₁-C₁₀ alkyl, C₆-C₁₀ aryl, trifluoromethyl, C₁-C₁₀ alkoxy, cyano, alkylenedioxy, C₁-C₁₀ alkylcarbonyl, and C₁-C₁₀ alkoxycarbonyl.

10. The compound, salt, stereoisomers, and mixtures comprising stereoisomers of claim 9, wherein R¹ is selected from 4-methylbenzyl, 4-chlorobenzyl, 4-trifluorobenzyl, phenyl, 4-phenylbenzyl, 4-iodobenzyl, 3-methoxybenzyl, 4-cyanobenzyl, 4-bromobenzyl, 2-methoxybenzyl, 4-fluorobenzyl, 4-methoxybenzyl, 2-phenylethyl, 4-methoxycarbonylbenzyl, and (benzo-1,4-dioxane-6-yl)methyl.

11. The compound, salt, stereoisomers, and mixtures comprising stereoisomers of claim 1, wherein R¹ is C₆₋₁₀ arylcarbonyl, C₁-C₁₀ alkylcarbonyl, or C₆₋₁₀ arylsulfonyl.

12.-13. (canceled)

15. The compound, salt, stereoisomers, and mixtures comprising stereoisomers of claim 1, wherein X is N and Y is CH.

16. The compound, salt, stereoisomers, and mixtures comprising stereoisomers of claim 15, wherein E, F, G, and H are all absent and o is 1.

17.-19. (canceled)

20. The compound, salt, stereoisomers, and mixtures comprising stereoisomers of claim 1, wherein Ar¹ is 4-chlorophenyl and Ar² is phenyl.

21. The compound, salt, stereoisomers, and mixtures comprising stereoisomers of claim 1, wherein R¹ is selected from methyl, ethyl, propyl, butyl, isopropyl, isobutyl, 2,2,2-tideuteroethyl, 2,2,2-trifluoroethyl, cyclopentyl, cyclohexyl, methylcarbonyl, (2,4-dimethoxyphenyl)methyl, 4-methylpiperazin-1-yl, 1-methylpiperidin-4-yl, 4-methylhomopiperazin-1-yl, —(CH₂)₂O(CH₂)₂OH, —CH₂CH₂OCH₂CH₂NH₂, and —(CH₂CH₂O)₄CH₂CH₂NH₂.

22-24. (canceled)

25. The compound, salt, stereoisomers, and mixtures comprising stereoisomers of claim 1, wherein E, F, G, and H are all absent o is 1, X is CH and Y is N, R¹ is hydrogen, methyl, or ethyl.

26. The compound, salt, stereoisomers, and mixtures comprising stereoisomers of claim 1, wherein the compound is a single enantiomer at the carbon bearing Ar¹ and Ar².

27. A pharmaceutical composition comprising a compound, salt, stereoisomers, and mixtures comprising stereoisomers of claim 1 and a pharmaceutically acceptable carrier.

28. A method of treating or preventing a viral infection in a mammal in need thereof comprising administering to a mammal in need thereof an effective amount of a compound of formula (I): 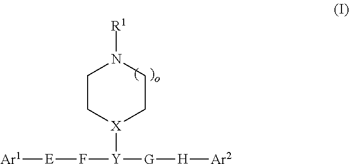
wherein R¹ is selected from hydrogen, C₁-C₁₀ alkyl, C₃-C₁₀ cycloalkyl, C₃-C₁₀ cycloalkyl C₁-C₁₀ alkyl, C₆-C₁₀ aryl, C₆-C₁₀ aryl C₁-C₁₀ alkyl, C₆-C₁₀ aryl C₃-C₁₀ cycloalkyl, heteroaryl, heterocyclyl, C₆₋₁₀ arylsulfonyl, C₆₋₁₀ arylcarbonyl, C₁-C₁₀ alkylcarbonyl, —(CH₂)ₓA(CH₂)_{y}B, and —(CH₂CH₂O)ₚ(CH₂CH₂)_{q}D, wherein the alkyl, aryl, or heteroaryl part of R¹ is optionally substituted with one or more substituents selected from deuterium, halo, C₁-C₁₀ alkyl, C₆-C₁₀ aryl, trifluoromethyl, C₁-C₁₀ alkoxy, cyano, alkylenedioxy, C₁-C₁₀ alkylcarbonyl, and C₁-C₁₀ alkoxycarbonyl,
Ar¹ and Ar² are the same or different and are independently selected from C₆-C₁₀ aryl, heteroaryl, and heterocyclyl, wherein the aryl, heteroaryl, and heterocyclyl are optionally substituted with one or more substituents selected from halo, C₁-C₁₀ alkyl, C₆-C₁₀ aryl, trifluoromethyl, C₁-C₁₀ alkoxy, C₁-C₁₀ alkylcarbonyl, and C₁-C₁₀ alkoxycarbonyl,
A is O, S, or N,
x and y are independently 1-4, inclusive,
B is selected from OR⁴, COOR⁵, and CONR⁶R⁷,
wherein R⁴, R⁵, R⁶, and R⁷ are independently selected from hydrogen, C₁-C₁₀ alkyl, C₃-C₁₀ cycloalkyl, and C₆-C₁₀ aryl,
D is NR⁸R⁹, OH, or OR¹²,
R⁸ and R⁹ are independently selected from hydrogen, COR¹⁰, and COOR¹¹,
R¹⁰ and R¹¹ are hydrogen or C₁-C₁₀ alkyl,
p and q are independently 1-4, inclusive,
E is absent or is (CR¹³R¹⁴)ₘ, NH, or S,
F is absent or is (CR¹⁵R¹⁶)ₙ, C═O, or —SO₂—,
G is absent or is (CR¹⁷CR¹⁸)ᵣ,
H is absent or is C═O, or —SO₂—,
m, n, and r are independently 0, 1, 2, 3, or 4,
o is 0, 1, or 2,
X and Y are independently CH or N,
or pharmaceutically acceptable salts, stereoisomers, and mixtures comprising stereoisomers thereof.

29.-58. (canceled)

59. A method for synergistically enhancing the antiviral effect of an anti-hepatitis C compound in a mammal undergoing treatment with the anti-hepatitis C compound, comprising co-administering to the mammal a compound of the formula (I): 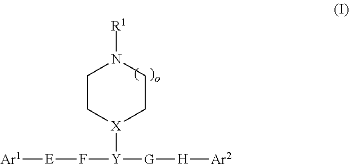
wherein R¹ is selected from hydrogen, C₁-C₁₀ alkyl, C₃-C₁₀ cycloalkyl, C₃-C₁₀ cycloalkyl C₁-C₁₀ alkyl, C₆-C₁₀ aryl, C₆-C₁₀ aryl C₁-C₁₀ alkyl, C₆-C₁₀ aryl C₃-C₁₀ cycloalkyl, heteroaryl, heterocyclyl, C₆₋₁₀ arylsulfonyl, C₆₋₁₀ arylcarbonyl, C₁-C₁₀ alkylcarbonyl, —(CH₂)ₓA(CH₂)_{y}B, and —(CH₂CH₂O)ₚ(CH₂CH₂)_{q}D, wherein the alkyl, aryl, or heteroaryl part of R¹ is optionally substituted with one or more substituents selected from deuterium, halo, C₁-C₁₀ alkyl, C₆-C₁₀ aryl, trifluoromethyl, C₁-C₁₀ alkoxy, cyano, alkylenedioxy, C₁-C₁₀ alkylcarbonyl, and C₁-C₁₀ alkoxycarbonyl,
Ar¹ and Ar² are the same or different and are independently selected from C₆-C₁₀ aryl, heteroaryl, and heterocyclyl, wherein the aryl, heteroaryl, and heterocyclyl are optionally substituted with one or more substituents selected from halo, C₁-C₁₀ alkyl, C₆-C₁₀ aryl, trifluoromethyl, C₁-C₁₀ alkoxy, C₁-C₁₀ alkylcarbonyl, and C₁-C₁₀ alkoxycarbonyl,
A is O, S, or N,
x and y are independently 1-4, inclusive,
B is selected from OR⁴, COOR⁵, and CONR⁶R⁷,
wherein R⁴, R⁵, R⁶, and R⁷ are independently selected from hydrogen, C₁-C₁₀ alkyl, C₃-C₁₀ cycloalkyl, and C₆-C₁₀ aryl,
D is NR⁸R⁹, OH, or OR¹²,
R⁸ and R⁹ are independently selected from hydrogen, COR¹⁰, and COOR¹¹,
R¹⁰ and R¹¹ are hydrogen or C₁-C₁₀ alkyl,
p and q are independently 1-4, inclusive,
E is absent or is (CR¹³R¹⁴)ₘ, NH, or S,
F is absent or is (CR¹⁵R¹⁶)ₙ, C═O, or —SO₂—,
G is absent or is (CR¹⁷CR¹⁸)ᵣ,
H is absent or is C═O, or —SO₂—,
m, n, and r are independently 0, 1, 2, 3, or 4,
o is 0, 1, or 2,
X and Y are independently CH or N,
or pharmaceutically acceptable salts, stereoisomers, and mixtures comprising stereoisomers thereof.

60. The method of claim 59, wherein the anti-hepatitis C compound is selected from include ribavirin, interferon-α, telaprevir, cyclosporin A, Asunaprevir (BMS-650032), Boceprevir, GS-9451, GS-9256, ABT-450, Danoprevir (RG7227), Faldaprevir (BI 201335), IDX320, MK-5172, Simeprevir (TMC435), Sovaprevir (ACH-1625), ABT-267, ACH-3102, BMS-791325, Daclatasvir (BMS-790052), GSK2336805, IDX719, JNJ-47910382, Ledipasvir (GS-5885), MK-8742, PPI-461, PPI-668, ABT-333, ALS-002200, BI 207127, IDX184, INX-08189, Mericitabine (RO5024048), PPI-383, PSI-352938, Setrobuvir (ANA-598), Sofosbuvir (PSI-7977 or GS-7977), Tegobuvir (GS-9190), TMC647055, Filibuvir (PF-00868554), GS-9669, GSK2878175, VX-135, VX-222, Algeron (Cepeginterferon Alfa-2b), BIP 48 (Peginterferon alfa 2b 48 kDA), Pegylated interferon alfa 2b, Pegylated interferon lambda (BMS-914143), Pegylated-P-Interferon-alpha-2b (P1101), and Alisporivir (DEB025).

61. A kit comprising:
(a) a compound of formula (I): 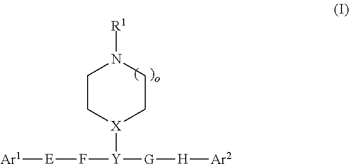
wherein R¹ is selected from hydrogen, C₁-C₁₀ alkyl, C₃-C₁₀ cycloalkyl, C₃-C₁₀ cycloalkyl C₁-C₁₀ alkyl, C₆-C₁₀ aryl, C₆-C₁₀ aryl C₁-C₁₀ alkyl, C₆-C₁₀ aryl C₃-C₁₀ cycloalkyl, heteroaryl, heterocyclyl, C₆₋₁₀ arylsulfonyl, C₆₋₁₀ arylcarbonyl, C₁-C₁₀ alkylcarbonyl, —(CH₂)ₓA(CH₂)_{y}B, and —(CH₂CH₂O)ₚ(CH₂CH₂)_{q}D, wherein the alkyl, aryl, or heteroaryl part of R¹ is optionally substituted with one or more substituents selected from deuterium, halo, C₁-C₁₀ alkyl, C₆-C₁₀ aryl, trifluoromethyl, C₁-C₁₀ alkoxy, cyano, alkylenedioxy, C₁-C₁₀ alkylcarbonyl, and C₁-C₁₀ alkoxycarbonyl,
Ar¹ and Ar² are the same or different and are independently selected from C₆-C₁₀ aryl, heteroaryl, and heterocyclyl, wherein the aryl, heteroaryl, and heterocyclyl are optionally substituted with one or more substituents selected from halo, C₁-C₁₀ alkyl, C₆-C₁₀ aryl, trifluoromethyl, C₁-C₁₀ alkoxy, C₁-C₁₀ alkylcarbonyl, and C₁-C₁₀ alkoxycarbonyl,
A is O, S, or N,
x and y are independently 1-4, inclusive,
B is selected from OR⁴, COOR⁵, and CONR⁶R⁷,
wherein R⁴, R⁵, R⁶, and R⁷ are independently selected from hydrogen, C₁-C₁₀ alkyl, C₃-C₁₀ cycloalkyl, and C₆-C₁₀ aryl,
D is NR⁸R⁹, OH, or OR¹²,
R⁸ and R⁹ are independently selected from hydrogen, COR¹⁰ and COOR¹¹,
R¹⁰ and R¹¹ are hydrogen or C₁-C₁₀ alkyl,
p and q are independently 1-4, inclusive,
E is absent or is (CR¹³R¹⁴)ₘ, NH, or S,
F is absent or is (CR¹⁵R¹⁶)ₙ, C═O, or —SO₂—,
G is absent or is (CR¹⁷CR¹⁸)ᵣ,
H is absent or is C═O, or —SO₂—,
m, n, and r are independently 0, 1, 2, 3, or 4,
o is 0, 1, or 2,
X and Y are independently CH or N,
or pharmaceutically acceptable salts, stereoisomers, and mixtures comprising stereoisomers thereof, and
(b) an anti-hepatitis C compound other than a compound of formula (I).

62.-66. (canceled)

67. A method of treating or preventing cancer in a mammal in need thereof comprising administering to a mammal in need thereof an effective amount of a compound of formula (I): 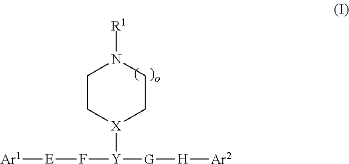
wherein R¹ is selected from hydrogen, C₁-C₁₀ alkyl, C₃-C₁₀ cycloalkyl, C₃-C₁₀ cycloalkyl C₁-C₁₀ alkyl, C₆-C₁₀ aryl, C₆-C₁₀ aryl C₁-C₁₀ alkyl, C₆-C₁₀ aryl C₃-C₁₀ cycloalkyl, heteroaryl, heterocyclyl, C₆₋₁₀ arylsulfonyl, C₆₋₁₀ arylcarbonyl, C₁-C₁₀ alkylcarbonyl, —(CH₂)ₓA(CH₂)_{y}B, and —(CH₂CH₂O)ₚ(CH₂CH₂)_{q}D, wherein the alkyl, aryl, or heteroaryl part of R¹ is optionally substituted with one or more substituents selected from deuterium, halo, C₁-C₁₀ alkyl, C₆-C₁₀ aryl, trifluoromethyl, C₁-C₁₀ alkoxy, cyano alkylenedioxy, C₁-C₁₀ alkylcarbonyl, and C₁-C₁₀ alkoxycarbonyl,
Ar¹ and Ar² are the same or different and are independently selected from C₆-C₁₀ aryl, heteroaryl, and heterocyclyl, wherein the aryl, heteroaryl, and heterocyclyl are optionally substituted with one or more substituents selected from halo, C₁-C₁₀ alkyl, C₆-C₁₀ aryl, trifluoromethyl, C₁-C₁₀ alkoxy, C₁-C₁₀ alkylcarbonyl, and C₁-C₁₀ alkoxycarbonyl,
A is O, S, or N,
x and y are independently 1-4, inclusive,
B is selected from OR⁴, COOR⁵, and CONR⁶R⁷,
wherein R⁴, R⁵, R⁶, and R⁷ are independently selected from hydrogen, C₁-C₁₀ alkyl, C₃-C₁₀ cycloalkyl, and C₆-C₁₀ aryl,
D is NR⁸R⁹, OH, or OR¹²,
R⁸ and R⁹ are independently selected from hydrogen, COR¹⁰, and COOR¹¹,
R¹⁰ and R¹¹ are hydrogen or C₁-C₁₀ alkyl,
p and q are independently 1-4, inclusive,
E is absent or is (CR¹³R¹⁴)ₘ, NH, or S,
F is absent or is (CR¹⁵R¹⁶)ₙ, C═O, or —SO₂—,
G is absent or is (CR¹⁷CR¹⁸)ᵣ,
H is absent or is C═O, or —SO₂—,
m, n, and r are independently 0, 1, 2, 3, or 4,
o is 0, 1, or 2,
X and Y are independently CH or N,
or pharmaceutically acceptable salts, stereoisomers, and mixtures comprising stereoisomers thereof.
